SEQUENCE LISTING (11 sequences)

SEQ ID NO: 1 - protein, 4 aa, Artificial Sequence
  Description of Artificial Sequence Synthetic peptide
KMDA

SEQ ID NO: 2 - protein, 4 aa, Artificial Sequence
  Description of Artificial Sequence Synthetic peptide
NLDA

SEQ ID NO: 3 - protein, 13 aa, Artificial Sequence
  Description of Artificial Sequence Synthetic peptide
SEVNLDAEFRCKK

SEQ ID NO: 4 - protein, 13 aa, Artificial Sequence
  Description of Artificial Sequence Synthetic peptide
SEVKMDAEFRCKK

SEQ ID NO: 5 - protein, 22 aa, Artificial Sequence
  Description of Artificial Sequence Synthetic peptide
GLNIKTEEISEISYEVEFRCKK

SEQ ID NO: 6 - protein, 34 aa, Artificial Sequence
  Description of Artificial Sequence Synthetic peptide
ADRGLTTRPGSGLTNIKTEEISEVNLDAEFRCKK

SEQ ID NO: 7 - protein, 33 aa, Artificial Sequence
  Description of Artificial Sequence Synthetic peptide
FVNQHLCGSHLVEALYLVCGERGFFYTPKACKK

SEQ ID NO: 8 - protein, 33 aa, Artificial Sequence
  Description of Artificial Sequence Synthetic peptide
CGGADRGLTTRPGSGLTNIKTEEISEVNLDAEF

SEQ ID NO: 9 - protein, 29 aa, Artificial Sequence
  Description of Artificial Sequence Synthetic peptide
CGGADRGLTTRPGSGLTNIKTEEISEVNL

SEQ ID NO: 10 - protein, 9 aa, Artificial Sequence
  Description of Artificial Sequence Synthetic peptide
SEVNLDAEF

SEQ ID NO: 11 - protein, 30 aa, Artificial Sequence
  Description of Artificial Sequence Synthetic peptide
ADRGLTTRPGSGLTNIKTEEISEVNLDAEF

(19) United States
(12) Patent Application Publication
(10) Pub. No.: US 2006/0014737 A1
(43) Pub. Date: Jan. 19, 2006

(54) Methods of treatment of amyloidosis using bi-aryl aspartyl protease inhibitors

(76) Inventors: Varghese John, San Francisco, CA (US); Roy Hom, San Francisco, CA (US); Jennifer Sealy, Oakland, CA (US); John Tucker, San Diego, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/075,445

(22) Filed: Mar. 9, 2005

Related U.S. Application Data
(60) Provisional application No. 60/551,205, filed on Mar. 9, 2004.
(60) Provisional application No. 60/551,013, filed on Mar. 9, 2004.
(60) Provisional application No. 60/575,964, filed on Jun. 2, 2004.
(60) Provisional application No. 60/575,859, filed on Jun. 2, 2004.
(60) Provisional application No. 60/591,906, filed on Jul. 29, 2004.
(60) Provisional application No. 60/591,856, filed on Jul. 29, 2004.
(60) Provisional application No. 60/614,035, filed on Sep. 30, 2004.
(60) Provisional application No. 60/614,060, filed on Sep. 30, 2004.

(51) Int. Cl. A61K 31/54; A61K 31/5377; A61K 31/495; A61K 31/138; A61K 31/445

(52) U.S. Cl. 514/227.5; 514/237.5; 514/255.03; 514/317; 514/651

(57) ABSTRACT

The invention relates to novel compounds and methods of treating diseases, disorders, and conditions associated with amyloidosis. Amyloidosis refers to a collection of diseases, disorders, and conditions associated with abnormal deposition of A-beta protein.

### CROSS-REFERENCE TO RELATED APPLICATIONS

This application claims the benefit of priority under 35 U.S.C. § 119(e) to U.S. Provisional Application 60/551,205 filed Mar. 9, 2004, U.S. Provisional Application 60/551,013 filed Mar. 9, 2004, U.S. Provisional Application 60/575,964 filed Jun. 2, 2004, U.S. Provisional Application 60/575,859 filed Jun. 2, 2004, U.S. Provisional Application 60/591,906 filed Jul. 29, 2004, U.S. Provisional Application 60/591,856 filed Jul. 29, 2004, U.S. Provisional Application 60/614,035 filed Sep. 30, 2004, and U.S. Provisional Application 60/614,060 filed Sep. 30, 2004.

### FIELD OF THE PRESENT INVENTION

The present invention is directed to novel compounds and also to methods of treating at least one condition, disorder, or disease associated with amyloidosis.

### BACKGROUND OF THE PRESENT INVENTION

Amyloidosis refers to a collection of at least one condition, disorder, or disease associated with abnormal deposition of amyloidal protein. For instance, Alzheimer's disease is believed to be caused by abnormal deposition of amyloidal protein in the brain. These amyloidal protein deposits, otherwise known as amyloid-beta peptide, A-beta, or betaA4, are the result of proteolytic cleavage of the amyloid precursor protein (APP).

The majority of APP molecules that undergo proteolytic cleavage are cleaved by the aspartyl protease alpha-secretase. Alpha-secretase cleaves APP between Lys687 and Leu688 producing a large, soluble fragment, alpha-sAPP, which is a secreted form of APP that does not result in beta-amyloid plaque formation. The alpha-secretase cleavage pathway precludes the formation of A-beta, thus providing an alternate target for preventing or treating amyloidosis.

Some APP molecules, however, are cleaved by a different aspartyl protease known as beta-secretase, which is also referred to in the literature as BACE, BACE1, Asp2, and Memapsin2. Beta-secretase cleaves APP after Met671, creating a C-terminal fragment. See, for example, Sinha et al., Nature, (1999), 402:537-554 and published PCT application WO 00/17369. After cleavage of APP by beta-secretase, an additional aspartyl protease, gamma-secretase, may then cleave the C-terminus of this fragment, at either Val711 or Ile713, found within the APP transmembrane domain, generating an A-beta peptide. The A-beta peptide may then proceed to form beta-amyloid plaques. A detailed description of the proteolytic processing of APP fragments is found, for example, in U.S. Pat. Nos. 5,441,870, 5,721,130, and 5,942,400.

The amyloidal disease Alzheimer's is a progressive degenerative disease that is characterized by two major pathologic observations in the brain which are (1) neurofibrillary tangles, and (2) beta-amyloid (or neuritic) plaques. A major factor in the development of Alzheimer's disease is A-beta deposits in regions of the brain responsible for cognitive activities. These regions include, for example, the hippocampus and cerebral cortex. A-beta is a neurotoxin that may be causally related to neuronal death observed in Alzheimer's disease patients. See, for example, Selkoe, Neuron, 6 (1991) 487. Since A-beta peptide accumulates as a result of APP processing by beta-secretase, inhibiting beta-secretase's activity is desirable for the treatment of Alzheimer's disease.

Dementia-characterized disorders also arise from A-beta accumulation in the brain including accumulation in cerebral blood vessels (known as vasculary amyloid angiopathy) such as in the walls of meningeal and parenchymal arterioles, small arteries, capillaries, and venules. A-beta may also be found in cerebrospinal fluid of both individuals with or without Alzheimer's disease. Additionally, neurofibrillary tangles similar to the ones observed in Alzheimer's patients can also be found in individuals without Alzheimer's disease. In this regard, a patient exhibiting symptoms of Alzheimer's due to A-beta deposits and neurofibrillary tangles in their cerebrospinal fluid may in fact be suffering from some other form of dementia. See, for example, Seubert et al., Nature, 359 (1992) 325-327. Examples of other forms of dementia where A-beta accumulation generates amyloidogenic plaques or results in vascular amyloid angiopathy include Trisomy 21 (Down's Syndrome), Hereditary Cerebral Hemorrhage with amyloidosis of the Dutch-Type (HCHWA-D), and other neurodegenerative disorders. Inhibiting beta-secretase is therefore not only desirable for the treatment of Alzheimer's, but also for the treatment of other conditions associated with amyloidosis.

Amyloidosis is also implicated in the pathophysiology of stroke. Cerebral amyloid angiopathy is a common feature of the brains of stroke patients exhibiting symptoms of dementia, focal neurological syndromes, or other signs of brain damage. See, for example, Corio et al., Neuropath Appl. Neurobiol., 22 (1996) 216-227. This suggests that production and deposition of A-beta may contribute to the pathology of Alzheimer's disease, stroke, and other diseases and conditions associated with amyloidosis. Accordingly, the inhibition of A-beta production is desirable for the treatment of Alzheimer's disease, stroke, and other diseases and conditions associated with amyloidosis.

Presently there are no known effective treatments for preventing, delaying, halting, or reversing the progression of Alzheimer's disease and other conditions associated with amyloidosis. Consequently, there is an urgent need for methods of treatment capable of preventing and treating conditions associated with amyloidosis including Alzheimer's disease.

Likewise, there is a need for compounds and methods of treatment that inhibit beta-secretase-mediated cleavage of APP. There is also a need for compounds and methods of treatment using compounds that are effective inhibitors of A-beta production, and/or are effective at reducing A-beta deposits or plaques, as well as methods of treatment capable of combating diseases and conditions characterized by amyloidosis, or A-beta deposits, or plaques.

There is also a need for methods of treating conditions associated with amyloidosis using compounds that are efficacious, bioavailable and/or selective for beta-secretase. An increase in efficacy, selectivity, and/or oral bioavailability may result in preferred, safer, less expensive products that are easier for patients to use.

There is also a need for methods of treating conditions associated with amyloidosis using compounds with characteristics that would allow them to cross the blood-brain barrier. Desirable characteristics include a low molecular weight and a high log P (increased log P=increased lipophilicity).

Generally, known aspartyl protease inhibitors are either incapable of crossing the blood-brain barrier or do so with great difficulty. Thus, these compounds are unsuitable for the treatment of the conditions described herein. Accordingly, there is a need for methods of treating conditions associated with amyloidosis using compounds that can readily cross the blood-brain barrier and inhibit beta-secretase.

There is also a need for a method of finding suitable compounds for inhibiting beta-secretase activity, inhibiting cleavage of APP, inhibiting production of A-beta, and/or reducing A-beta deposits or plaques.

The present invention is directed to novel compounds and methods of treating at least one condition, disorder, or disease associated with amyloidosis. An embodiment of the present invention is a method of administering at least one compound of formula (I) 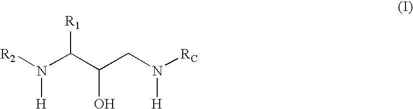
wherein R₁, R₂, and R_{C} are defined below, in treating at least one condition, disorder, or disease associated with amyloidosis. Another embodiment of the present invention is directed to methods of treatment comprising administering at least one compound of formula (I), wherein R₁, R₂, and R_{C} are defined below, useful in preventing, delaying, halting, or reversing the progression of Alzheimer's disease.

Another embodiment of the present invention is directed to uses of beta-secretase inhibitors of at least one compound of formula (I), wherein R₁, R₂, and R_{C} are defined below, in treating or preventing at least one condition, disorder, or disease associated with amyloidosis.

Another embodiment of the present invention is to administer beta-secretase inhibitors of at least one compound of formula (I), wherein R₁, R₂, and R_{C} are defined below, exhibiting at least one property chosen from improved efficacy, oral bioavailability, selectivity, and blood-brain barrier penetrating properties.

### BRIEF SUMMARY OF THE PRESENT INVENTION

The present invention is directed to novel compounds and methods of treating diseases, disorders, and conditions associated with amyloidosis. As previously noted, amyloidosis refers to a collection of diseases, disorders, and conditions associated with abnormal deposition of A-beta protein.

An embodiment of the present invention is to provide compounds having properties contributing to viable pharmaceutical compositions. These properties include improved efficacy, bioavailability, selectivity, and/or blood-brain barrier penetrating properties. They can be inter-related, though an increase in any one of them correlates to a benefit for the compound and its corresponding method of treatment. For example, an increase in any one of these properties may result in preferred, safer, less expensive products that are easier for patients to use.

In an embodiment, the present invention provides a method of preventing or treating conditions associated with amyloidosis, comprising administering to a patient in need thereof a composition comprising a therapeutically effective amount of at least one compound of formula (I), 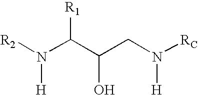
or pharmaceutically acceptable salts thereof, wherein R₁, R₂, and R_{C} are as defined below.

In another embodiment, the present invention provides a method of preventing or treating conditions associated with amyloidosis, comprising administering to a host a composition comprising a therapeutically effective amount of at least one compound of formula (I), or a pharmaceutically acceptable salt thereof, wherein the inhibition is at least 10% for a dose ≦100 mg/kg, and wherein R₁, R₂, and R_{C} are as defined below.

In another embodiment, the present invention provides a method for preventing or treating conditions associated with amyloidosis, comprising administering to a host a therapeutically effective amount of at least one compound of formula (I), or a pharmaceutically acceptable salt thereof, the compound having an F value of at least 10%, wherein R₁, R₂, and R_{C} are as defined below.

In another embodiment, the present invention provides a method of preventing or treating conditions associated with amyloidosis, comprising administering to a host a composition comprising a therapeutically effective amount of at least one selective beta-secretase inhibitor of formula (I), or pharmaceutically acceptable salt thereof, wherein R₁, R₂, and R_{C} are as defined below.

In another embodiment, the present invention provides a method of preventing or treating Alzheimer's disease by administering to a host an effective amount of at least one compound of formula (I), or a pharmaceutically acceptable salt thereof, wherein R₁, R₂, and R_{C} are as defined below.

In another embodiment, the present invention provides a method of preventing or treating dementia by administering to a host an effective amount of at least one compound of formula (I), or pharmaceutically acceptable salt thereof, wherein R₁, R₂, and R_{C} are as defined below.

In another embodiment, the present invention provides a method of inhibiting beta-secretase activity in a host, the method comprising administering to the host an effective amount of at least one compound of formula (I), or a pharmaceutically acceptable salt thereof, wherein R₁, R₂, and R_{C} are as defined below.

In another embodiment, the present invention provides a method of inhibiting beta-secretase activity in a cell, the method comprising administering to the cell an effective amount of at least one compound of formula (I), or a pharmaceutically acceptable salt thereof, wherein R₁, R₂, and R_{C} are as defined below.

In another embodiment, the present invention provides a method of inhibiting beta-secretase activity in a host, the method comprising administering to the host an effective amount of at least one compound of formula (I), or a pharmaceutically acceptable salt thereof, wherein the host is a human, and wherein R₁, R₂, and R_{C} are as defined below.

In another embodiment, the present invention provides a method of affecting beta-secretase-mediated cleavage of amyloid precursor protein in a patient, comprising administering a therapeutically effective amount of at least one compound of formula (I), or a pharmaceutically acceptable salt thereof, wherein R₁, R₂, and R_{C} are as defined below.

In another embodiment, the present invention provides a method of inhibiting cleavage of amyloid precursor protein at a site between Met596 and Asp597 (numbered for the APP-695 amino acid isotype), or at a corresponding site of an isotype or mutant thereof, comprising administering a therapeutically effective amount of at least one compound of formula (I), or a pharmaceutically acceptable salt thereof, wherein R₁, R₂, and R_{C} are as defined below.

In another embodiment, the present invention provides a method of inhibiting production of A-beta, comprising administering to a patient a therapeutically effective amount of at least one compound of formula (I), or a pharmaceutically acceptable salt thereof, wherein R₁, R₂, and R_{C} are as defined below.

In another embodiment, the present invention provides a method of preventing or treating deposition of A-beta, comprising administering a therapeutically effective amount of at least one compound of formula (I), or a pharmaceutically acceptable salt thereof, wherein R₁, R₂, and R_{C} are as defined below.

In another embodiment, the present invention provides a method of preventing, delaying, halting, or reversing a disease characterized by A-beta deposits or plaques, comprising administering a therapeutically effective amount of at least one compound of formula (I), or a pharmaceutically acceptable salt thereof, wherein R₁, R₂, and R_{C} are as defined below.

In another embodiment, the A-beta deposits or plaques are in a human brain.

In another embodiment, the present invention provides a method of inhibiting the activity of at least one aspartyl protease in a patient in need thereof, comprising administering a therapeutically effective amount of at least one compound of formula (I), or a pharmaceutically acceptable salt thereof, wherein R₁, R₂, and R_{C} are as defined below.

In another embodiment, the at least one aspartyl protease is beta-secretase.

In another embodiment, the present invention provides a method of interacting an inhibitor with beta-secretase, comprising administering to a patient in need thereof a therapeutically effective amount of at least one compound of formula (I), or a pharmaceutically acceptable salt thereof, wherein R₁, R₂, and R_{C} are as defined below, wherein the at least one compound interacts with at least one beta-secretase subsite such as S1, S1′, or S2′.

In another embodiment, the present invention provides an article of manufacture, comprising (a) at least one dosage form of at least one compound of formula (I), or pharmaceutically acceptable salt thereof, wherein R₁, R₂, and R_{C} are defined below, (b) a package insert providing that a dosage form comprising a compound of formula (I) should be administered to a patient in need of therapy for at least one disorder, condition or disease associated with amyloidosis, and (c) at least one container in which at least one dosage form of at least one compound of formula (I) is stored.

In another embodiment, the present invention provides a packaged pharmaceutical composition for treating conditions related to amyloidosis, comprising (a) a container which holds an effective amount of at least one compound of formula (I), or a pharmaceutically acceptable salt thereof, wherein R₁, R₂, and R_{C} are as defined below, and (b) instructions for using the pharmaceutical composition.

### Definitions

Throughout the specification and claims, including the detailed description below, the following definitions apply.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

Where multiple substituents are indicated as being attached to a structure, the substituents can be the same or different.

APP, amyloid precursor protein, is defined as any APP polypeptide, including APP variants, mutations, and isoforms, for example, as disclosed in U.S. Pat. No. 5,766,846.

Beta-amyloid peptide (A-beta peptide) is defined as any peptide resulting from beta-secretase mediated cleavage of APP, including, for example, peptides of 39, 40, 41, 42, and 43 amino acids, and extending from the beta-secretase cleavage site to amino acids 39, 40, 41, 42, or 43.

Beta-secretase is an aspartyl protease that mediates cleavage of APP at the N-terminus of A-beta. Human beta-secretase is described, for example, in WO 00/17369.

The term “complex” as used herein refers to an inhibitor-enzyme complex, wherein the inhibitor is a compound of formula (I) described herein, and wherein the enzyme is beta-secretase or a fragment thereof.

The term “host” as used herein refers to a cell or tissue, in vitro or in vivo, an animal, or a human.

The term “treating” refers to administering a compound or a composition of formula (I) to a host having at least a tentative diagnosis of disease or condition. The methods of treatment and compounds of the present invention will delay, halt, or reverse the progression of the disease or condition thereby giving the host a longer and/or more functional life span.

The term “preventing” refers to administering a compound or a composition of formula (I) to a host who has not been diagnosed as having the disease or condition at the time of administration, but who could be expected to develop the disease or condition or be at increased risk for the disease or condition. The methods of treatment and compounds of the present invention may slow the development of disease symptoms, delay the onset of the disease or condition, halt the progression of disease development, or prevent the host from developing the disease or condition at all. Preventing also includes administration of a compound or a composition of the present invention to those hosts thought to be predisposed to the disease or condition due to age, familial history, genetic or chromosomal abnormalities, due to the presence of one or more biological markers for the disease or condition, such as a known genetic mutation of APP or APP cleavage products in brain tissues or fluids, and/or due to environmental factors.

The term “halogen” in the present invention refers to fluorine, bromine, chlorine, or iodine.

The term “alkyl” in the present invention refers to straight or branched chain alkyl groups having 1 to 20 carbon atoms. An alkyl group may optionally comprise at least one double bond and/or at least one triple bond. The alkyl groups herein are unsubstituted or substituted in one or more positions with various groups. For example, such alkyl groups may be optionally substituted with at least one group selected from alkyl, alkoxy, —C(O)H, carboxy, alkoxycarbonyl, cycloalkyl, heterocycloalkyl, aryl, heteroaryl, amido, alkanoylamino, amidino, alkoxycarbonylamino, N-alkyl amidino, N-alkyl amido, N,N′-dialkylamido, aralkoxycarbonylamino, halogen, alkyl thio, alkylsulfinyl, alkylsulfonyl, hydroxy, cyano, nitro, amino, monoalkylamino, dialkylamino, halo alkyl, halo alkoxy, aminoalkyl, monoalkylaminoalkyl, dialkylaminoalkyl, and the like. Additionally, at least one carbon within any such alkyl may be optionally replaced with —C(O)—.

Examples of alkyls include methyl, ethyl, ethenyl, ethynyl, propyl, 1-ethyl-propyl, propenyl, propynyl, isopropyl, n-butyl, isobutyl, sec-butyl, tert-butyl, 2-methylbutyl, 3-methyl-butyl, 1-but-3-enyl, butynyl, pentyl, 2-pentyl, isopentyl, neopentyl, 3-methylpentyl, 1-pent-3-enyl, 1-pent-4-enyl, pentyn-2-yl, hexyl, 2-hexyl, 3-hexyl, 1-hex-5-enyl, formyl, acetyl, acetylamino, trifluoromethyl, propionic acid ethyl ester, trifluoroacetyl, methylsulfonyl, ethylsulfonyl, 1-hydroxy-1,1-methylethyl, 2-hydroxy-1,1-dimethyl-ethyl, 1,1-dimethyl-propyl, cyano-dimethyl-methyl, propylamino, and the like.

In an embodiment, alkyls may be selected from the group comprising sec-butyl, isobutyl, ethynyl, 1-ethyl-propyl, pentyl, 3-methyl-butyl, pent-4-enyl, isopropyl, tert-butyl, 2-methylbutane, and the like.

In another embodiment, alkyls may be selected from formyl, acetyl, acetylamino, trifluoromethyl, propionic acid ethyl ester, trifluoroacetyl, methylsulfonyl, ethylsulfonyl, 1-hydroxy-1-methylethyl, 2-hydroxy-1,1-dimethyl-ethyl, 1,1-dimethyl-propyl, cyano-dimethyl-methyl, propylamino, and the like.

The term “alkoxy” in the present invention refers to straight or branched chain alkyl groups, wherein an alkyl group is as defined above, and having 1 to 20 carbon atoms, attached through at least one divalent oxygen atom, such as, for example, methoxy, ethoxy, propoxy, isopropoxy, n-butoxy, sec-butoxy, tert-butoxy, pentoxy, isopentoxy, neopentoxy, hexyloxy, heptyloxy, allyloxy, 2-(2-methoxy-ethoxy)-ethoxy, benzyloxy, 3-methylpentoxy, and the like.

In an embodiment, alkoxy groups may be selected from the group comprising allyloxy, hexyloxy, heptyloxy, 2-(2-methoxy-ethoxy)-ethoxy, benzyloxy, and the like.

The term “—C(O)-alkyl” or “alkanoyl” refers to an acyl radical derived from an alkylcarboxylic acid, a cycloalkylcarboxylic acid, a heterocycloalkylcarboxylic acid, an arylcarboxylic acid, an arylalkylcarboxylic acid, a heteroarylcarboxylic acid, or a heteroarylalkylcarboxylic acid, examples of which include formyl, acetyl, 2,2,2-trifluoroacetyl, propionyl, butyryl, valeryl, 4-methylvaleryl, and the like.

The term “cycloalkyl” refers to an optionally substituted carbocyclic ring system of one or more 3, 4, 5, 6, 7, or 8 membered rings. A cycloalkyl can further include 9, 10, 11, 12, 13, and 14 membered fused ring systems. A cycloalkyl can be saturated or partially unsaturated. A cycloalkyl may be monocyclic, bicyclic, tricyclic, and the like. Bicyclic and tricyclic as used herein are intended to include both fused ring systems, such as adamantyl, octahydroindenyl, decahydro-naphthyl, and the like, substituted ring systems, such as cyclopentylcyclohexyl, and spirocycloalkyls such as spiro[2.5]octane, spiro[4.5]decane, 1,4-dioxa-spiro[4.5]decane, and the like. A cycloalkyl may optionally be a benzo fused ring system, which is optionally substituted as defined herein with respect to the definition of aryl. At least one —CH₂— group within any such cycloalkyl ring system may be optionally replaced with —C(O)—, —C(S)—, —C(═N—H)—, —C(═N—OH)—, —C(═N-alkyl)-(optionally substituted as defined herein with respect to the definition of alkyl), or —C(═N—O-alkyl)-(optionally substituted as defined herein with respect to the definition of alkyl).

Further examples of cycloalkyl radicals include cyclopropyl, cyclobutyl, cyclopentyl, cyclohexyl, octahydronaphthyl, 2,3-dihydro-1H-indenyl, and the like.

In an embodiment, a cycloalkyl may be selected from the group comprising cyclopentyl, cyclohexyl, cycloheptyl, adamantenyl, bicyclo[2.2.1]heptyl, and the like.

The cycloalkyl groups herein are unsubstituted or substituted in at least one position with various groups. For example, such cycloalkyl groups may be optionally substituted with alkyl, alkoxy, —C(O)H, carboxy, alkoxycarbonyl, cycloalkyl, heterocycloalkyl, aryl, heteroaryl, amido, alkanoylamino, amidino, alkoxycarbonylamino, N-alkyl amidino, N-alkyl amido, N,N′-dialkylamido, aralkoxycarbonylamino, halogen, alkylthio, alkylsulfinyl, alkylsulfonyl, hydroxy, cyano, nitro, amino, monoalkylamino, dialkylamino, haloalkyl, haloalkoxy, aminoalkyl, monoalkylaminoalkyl, dialkylaminoalkyl, and the like.

The term “cycloalkylcarbonyl” refers to an acyl radical of the formula cycloalkyl-C(O)— in which the term “cycloalkyl” has the significance given above, such as cyclopropylcarbonyl, cyclohexylcarbonyl, adamantylcarbonyl, 1,2,3,4-tetrahydro-2-naphthoyl, 2-acetamido-1,2,3,4-tetrahydro-2-naphthoyl, 1-hydroxy-1,2,3,4-tetrahydro-6-naphthoyl, and the like.

The term “heterocycloalkyl,” “heterocycle,” or “heterocyclyl,” refers to a monocyclic, bicyclic, or tricyclic heterocycle radical, containing at least one nitrogen, oxygen or sulfur atom ring member and having 3 to 8 ring members in each ring, wherein at least one ring in the heterocycloalkyl ring system may optionally contain at least one double bond. At least one —CH₂— group within any such heterocycloalkyl ring system may be optionally replaced with —C(O)—, —C(S)—, —C(═N—H)—, —C(═N—OH)—, —C(═N-alkyl)— (optionally substituted as defined herein with respect to the definition of alkyl), or —C(═N—O-alkyl)— (optionally substituted as defined herein with respect to the definition of alkyl).

The terms “bicyclic” and “tricyclic” as used herein are intended to include both fused ring systems, such as 2,3-dihydro-1H-indole, and substituted ring systems, such as bicyclohexyl. At least one —CH₂— group within any such heterocycloalkyl ring system may be optionally replaced with —C(O)—, —C(N)— or —C(S)—. Heterocycloalkyl is intended to include sulfones, sulfoxides, N-oxides of tertiary nitrogen ring members, and carbocyclic fused and benzo fused ring systems wherein the benzo fused ring system is optionally substituted as defined herein with respect to the definition of aryl. Such heterocycloalkyl radicals may be optionally substituted on one or more carbon atoms by halogen, alkyl, alkoxy, cyano, nitro, amino, alkylamino, dialkylamino, monoalkylaminoalkyl, dialkylaminoalkyl, haloalkyl, haloalkoxy, aminohydroxy, oxo, aryl, aralkyl, heteroaryl, heteroaralkyl, amidino, N-alkylamidino, alkoxycarbonylamino, alkylsulfonylamino, and the like, and/or on a secondary nitrogen atom (i.e., —NH—) by hydroxy, alkyl, aralkoxycarbonyl, alkanoyl, heteroaralkyl, phenyl, phenylalkyl, and the like.

Examples of a heterocycloalkyl include morpholinyl, thiomorpholinyl, thiomorpholinyl S-oxide, thiomorpholinyl S,S-dioxide, piperazinyl, homopiperazinyl, pyrrolidinyl, pyrrolinyl, 2,5-dihydro-pyrrolyl, tetrahydropyranyl, pyranyl, thiopyranyl, piperidinyl, tetrahydrofuranyl, tetrahydrothienyl, imidazolidinyl, homopiperidinyl, 1,2dihyrdo-pyridinyl, homomorpholinyl, homothiomorpholinyl, homothiomorpholinyl S,S-dioxide, oxazolidinonyl, dihydropyrazolyl, dihydropyrrolyl, 1,4-dioxa-spiro[4.5]decyl, dihydropyrazinyl, dihydropyridinyl, dihydropyrimidinyl, dihydrofuryl, dihydropyranyl, tetrahydrothienyl S-oxide, tetrahydrothienyl S,S-dioxide, homothiomorpholinyl S-oxide, 2-oxo-piperidinyl, 5-oxo-pyrrolidinyl, 2-oxo-1,2-dihydro-pyridinyl, 6-oxo-6H-pyranyl, 1,1-dioxo-hexahydro-thiopyranyl, 1-acetyl-piperidinyl, 1-methanesulfonylpiperidinyl, 1-ethanesulfonylpiperidinyl, 1-oxo-hexahydro-thiopyranyl, 1-(2,2,2-trifluoroacetyl)-piperidinyl, 1-formyl-piperidinyl, and the like.

In an embodiment, a heterocycloalkyl may be selected from pyrrolidinyl, 2,5-dihydro-pyrrolyl, piperidinyl, 1,2-dihyrdo-pyridinyl, pyranyl, piperazinyl, imidazolidinyl, thiopyranyl, tetrahydropyranyl, 1,4-dioxa-spiro[4.5]decyl, and the like.

In another embodiment, a heterocycloalkyl may be selected from 2-oxo-piperidinyl, 5-oxo-pyrrolidinyl, 2-oxo-1,2-dihydro-pyridinyl, 6-oxo-6H-pyranyl, 1,1-dioxo-hexahydro-thiopyranyl, 1-acetyl-piperidinyl, 1-methanesulfonyl piperidinyl, 1-ethanesulfonylpiperidinyl, 1-oxo-hexahydro-thiopyranyl, 1-(2,2,2-trifluoroacetyl)-piperidinyl, 1-formyl-piperidinyl, and the like.

The term “aryl” refers to an aromatic carbocyclic group having a single ring (e.g., phenyl) or multiple condensed rings in which at least one ring is aromatic. The aryl may be monocyclic, bicyclic, tricyclic, etc. Bicyclic and tricyclic as used herein are intended to include both fused ring systems, such as naphthyl and β-carbolinyl, and substituted ring systems, such as biphenyl, phenylpyridyl, diphenylpiperazinyl, tetrahydronaphthyl, and the like. Preferred aryl groups of the present invention are phenyl, 1-naphthyl, 2-naphthyl, indanyl, indenyl, dihydronaphthyl, fluorenyl, tetralinyl or 6,7,8,9-tetrahydro-5H-benzo[a]cycloheptenyl. The aryl groups herein are unsubstituted or substituted in one or more positions with various groups. For example, such aryl groups may be optionally substituted with alkyl, alkoxy, —C(O)H, carboxy, alkoxycarbonyl, aryl, heteroaryl, cycloalkyl, heterocycloalkyl, amido, alkanoylamino, amidino, alkoxycarbonylamino, N-alkyl amidino, N-alkyl amido, N,N′-dialkylamido, aralkoxycarbonylamino, halogen, alkyl thio, alkylsulfinyl, alkylsulfonyl, hydroxy, cyano, nitro, amino, monoalkylamino, dialkylamino, aralkoxycarbonylamino, halo alkyl, halo alkoxy, aminoalkyl, monoalkylaminoalkyl, dialkylaminoalkyl, and the like.

Examples of aryl radicals are phenyl, p-tolyl, 4-methoxyphenyl, 4-(tert-butoxy)phenyl, 3-methyl-4-methoxyphenyl, 4-CF₃-phenyl, 4-fluorophenyl, 4-chlorophenyl, 3-nitrophenyl, 3-aminophenyl, 3-acetamidophenyl, 4-acetamidophenyl, 2-methyl-3-acetam idophenyl, 2-methyl-3-aminophenyl, 3-methyl-4-aminophenyl, 2-amino-3-methylphenyl, 2,4-dimethyl-3-aminophenyl, 4-hydroxyphenyl, 3-methyl-4-hydroxyphenyl, 1-naphthyl, 2-naphthyl, 3-amino-1-naphthyl, 2-methyl-3-amino-1-naphthyl, 6-amino-2-naphthyl, 4,6-dimethoxy-2-naphthyl, piperazinylphenyl, and the like.

Further examples of aryl radicals include 3-tert-butyl-1-fluoro-phenyl, 1,3-difluoro-phenyl, (1-hydroxy-1-methyl-ethyl)-phenyl, 1-fluoro-3-(2-hydroxy-1, 1-dimethyl-ethyl)-phenyl, (1,1-dimethyl-propyl)-phenyl, cyclobutyl-phenyl, pyrrolidin-2-yl-phenyl, (5-oxo-pyrrolidin-2-yl)-phenyl, (2,5-dihydro-1H-pyrrol-2-yl)-phenyl, (1H-pyrrol-2-yl)-phenyl, (cyano-dimethyl-methyl)-phenyl, tert-butyl-phenyl, 1-fluoro-2-hydroxy-phenyl, 1,3-difluoro-4-propylamino-phenyl, 1,3-difluoro-4-hydroxy-phenyl, 1,3-difluoro-4-ethylamino-phenyl, 3-isopropyl-phenyl, (3H-[1,2,3]triazol-4-yl)-phenyl, [1,2,3]triazol-1-yl-phenyl, [1,2,4]thiadiazol-3-yl-phenyl, [1,2,4]thiadiazol-5-yl-phenyl, (4H-[1,2,4]triazol-3-yl)-phenyl, [1,2,4]oxadiazol-3-yl-phenyl, imidazol-1-yl-phenyl, (3H-imidazol-4-yl)-phenyl, [1,2,4]triazol-4-yl-phenyl, [1,2,4]oxadiazol-5-yl-phenyl, isoxazol-3-yl-phenyl, (1-methyl-cyclopropyl)-phenyl, isoxazol-4-yl-phenyl, isoxazol-5-yl-phenyl, 1-cyano-2-tert-butyl-phenyl, 1-trifluoromethyl-2-tert-butyl-phenyl, 1-chloro-2-tert-butyl-phenyl, 1-acetyl-2-tert-butyl-phenyl, 1-tert-butyl-2-methyl-phenyl, 1-tert-butyl-2-ethyl-phenyl, 1-cyano-3-tert-butyl-phenyl, 1-trifluoromethyl-3-tert-butyl-phenyl, 1-chloro-3-tert-butyl-phenyl, 1-acetyl-3-tert-butyl-phenyl, 1-tert-butyl-3-methyl-phenyl, 1-tert-butyl-3-ethyl-phenyl, 4-tert-butyl-1-imidazol-1-yl-phenyl, ethylphenyl, isobutyl-phenyl, isopropylphenyl, 3-allyloxy-1-fluoro-phenyl, (2,2-dimethyl-propyl)-phenyl, ethynylphenyl, 1-fluoro-3-heptyloxy-phenyl, 1-fluoro-3-[2-(2-methoxy-ethoxy)-ethoxy]-phenyl, 1-benzyloxy-3-fluoro-phenyl, 1-fluoro-3-hydroxy-phenyl, 1-fluoro-3-hexyloxy-phenyl, (4-methyl-thiophen-2-yl)-phenyl, (5-acetyl-thiophen-2-yl)-phenyl, furan-3-yl-phenyl, thiophen-3-yl-phenyl, (5-formyl-thiophen-2-yl)-phenyl, (3-formyl-furan-2-yl)-phenyl, acetylamino-phenyl, trifluoromethylphenyl, sec-butyl-phenyl, pentylphenyl, (3-methyl-butyl)-phenyl, (1-ethyl-propyl)-phenyl, cyclopentyl-phenyl, 3-pent-4-enyl-phenyl, phenyl propionic acid ethyl ester, pyridin-2-yl-phenyl, (3-methyl-pyridin-2-yl)-phenyl, thiazol-2-yl-phenyl, (3-methyl-thiophen-2-yl)-phenyl, fluoro-phenyl, adamantan-2-yl-phenyl, 1,3-difluoro-2-hydroxy-phenyl, cyclopropyl-phenyl, 1-bromo-3-tert-butyl-phenyl, (3-bromo-[1,2,4]thiadiazol-5-yl)-phenyl, (1-methyl-1H-imidazol-2-yl)-phenyl, (3,5-dimethyl-3H-pyrazol-4-yl)-phenyl, (3,6-dimethyl-pyrazin-2-yl)-phenyl, (3-cyano-pyrazin-2-yl)-phenyl, thiazol-4-yl-phenyl, (4-cyano-pyridin-2-yl)-phenyl, pyrazin-2-yl-phenyl, (6-methyl-pyridazin-3-yl)-phenyl, (2-cyano-thiophen-3-yl)-phenyl, (2chloro-thiophen-3-yl)-phenyl, (5-acetyl-thiophen-3-yl)-phenyl, cyano-phenyl, and the like.

The term “heteroaryl” refers to an aromatic heterocycloalkyl radical as defined above. The heteroaryl groups herein are unsubstituted or substituted in at least one position with various groups. For example, such heteroaryl groups may be optionally substituted with, for example, alkyl, alkoxy, halogen, hydroxy, cyano, nitro, amino, monoalkylamino, dialkylamino, haloalkyl, haloalkoxy, —C(O)H, carboxy, alkoxycarbonyl, cycloalkyl, heterocycloalkyl, aryl, heteroaryl, amido, alkanoylamino, amidino, alkoxycarbonylamino, N-alkyl amidino, N-alkyl amido, N,N′-dialkylamido, alkyl thio, alkylsulfinyl, alkylsulfonyl, aralkoxycarbonylamino, aminoalkyl, monoalkylaminoalkyl, dialkylaminoalkyl, and the like.

Examples of heteroaryl groups include pyridyl, pyrimidyl, furanyl, imidazolyl, thienyl, oxazolyl, thiazolyl, pyrazinyl, 3-methyl-thienyl, 4-methyl-thienyl, 3-propyl-thienyl, 2-chloro-thienyl, 2-chloro-4-ethyl-thienyl, 2-cyano-thienyl, 5-acetyl-thienyl, 5-formyl-thienyl, 3-formyl-furanyl, 3-methyl-pyridinyl, 3-bromo-[1,2,4]thiadiazolyl, 1-methyl-1H-imidazole, 3,5-dimethyl-3H-pyrazolyl, 3,6-dimethyl-pyrazinyl, 3-cyano-pyrazinyl, 4-tert-butyl-pyridinyl, 4-cyano-pyridinyl, 6-methyl-pyridazinyl, 2-tert-butyl-pyrimidinyl, 4-tert-butyl-pyrimidinyl, 6-tert-butyl-pyrimidinyl, 5-tert-butyl-pyridazinyl, 6-tert-butyl-pyridazinyl, quinolinyl, benzothienyl, indolyl, indolinyl, pyridazinyl, isoindolyl, isoquinolyl, quinazolinyl, quinoxalinyl, phthalazinyl, imidazolyl, isoxazolyl, pyrazolyl, indolizinyl, indazolyl, benzothiazolyl, benzimidazolyl, benzofuranyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromenyl, chromenyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazoth iazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromenonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide, tetrahydrocarbazole, tetrahydrobetacarboline, and the like.

In an embodiment, a heteroaryl group may be selected from pyridyl, pyrimidyl, furanyl, imidazolyl, thienyl, oxazolyl, thiazolyl, pyrazinyl, and the like.

In another embodiment, a heteroaryl group may be selected from 3-methyl-thienyl, 4-methyl-thienyl, 3-propyl-thienyl, 2-chloro-thienyl, 2-chloro-4-ethyl-thienyl, 2-cyano-thienyl, 5-acetyl-thienyl, 5-formyl-thienyl, 3-formyl-furanyl, 3-methyl-pyridinyl, 3-bromo-[1,2,4]thiadiazolyl, 1-methyl-1H-imidazole, 3,5-dimethyl-3H-pyrazolyl, 3,6-dimethyl-pyrazinyl, 3-cyano-pyrazinyl, 4-tert-butyl-pyridinyl, 4-cyano-pyridinyl, 6-methyl-pyridazinyl, 2-tert-butyl-pyrimidinyl, 4-tert-butyl-pyrimidinyl, 6-tert-butyl-pyrimidinyl, 5-tert-butyl-pyridazinyl, 6-tert-butyl-pyridazinyl, and the like.

Further examples of heterocycloalkyls and heteroaryls may be found in Katritzky, A. R. et al., Comprehensive Heterocyclic Chemistry: The Structure, Reactions, Synthesis and Use of Heterocyclic Compounds, Vol. 1-8, New York: Pergamon Press, 1984.

The term “aralkoxycarbonyl” refers to a radical of the formula aralkyl-O—C(O)— in which the term “aralkyl” is encompassed by the definitions above for aryl and alkyl. Examples of an aralkoxycarbonyl radical include benzyloxycarbonyl, 4-methoxyphenylmethoxycarbonyl, and the like.

The term “aryloxy” refers to a radical of the formula —O-aryl in which the term aryl is as defined above.

The term “aralkanoyl” refers to an acyl radical derived from an aryl-substituted alkanecarboxylic acid such as phenylacetyl, 3-phenylpropionyl(hydrocinnamoyl), 4-phenylbutyryl, (2-naphthyl)acetyl, 4-chlorohydrocinnamoyl, 4-aminohydrocinnamoyl, 4-methoxyhydrocinnamoyl, and the like.

The term “aroyl” refers to an acyl radical derived from an arylcarboxylic acid, “aryl” having the meaning given above. Examples of such aroyl radicals include substituted and unsubstituted benzoyl or naphthoyl such as benzoyl, 4-chlorobenzoyl, 4-carboxybenzoyl, 4-(benzyloxycarbonyl)benzoyl, 1-naphthoyl, 2-naphthoyl, 6-carboxy-2 naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The term “haloalkyl” refers to an alkyl radical having the meaning as defined above wherein one or more hydrogens are replaced with a halogen. Examples of such haloalkyl radicals include chloromethyl, 1-bromoethyl, fluoromethyl, difluoromethyl, trifluoromethyl, 1,1,1-trifluoroethyl, and the like.

The term “epoxide” refers to chemical compounds or reagents comprising a bridging oxygen wherein the bridged atoms are also bonded to one another either directly or indirectly. Examples of epoxides include epoxyalkyl (e.g., ethylene oxide and 1,2-epoxybutane), epoxycycloalkyl (e.g., 1,2-epoxycyclohexane and 1,2-epoxy-1-methylcyclohexane), and the like.

The term “structural characteristics” refers to chemical moieties, chemical motifs, and portions of chemical compounds. These include R groups, such as those defined herein, ligands, appendages, and the like. For example, structural characteristics may be defined by their properties, such as, but not limited to, their ability to participate in intermolecular interactions, including Van der Waal's (e.g., electrostatic interactions, dipole-dipole interactions, dispersion forces, hydrogen bonding, and the like). Such characteristics may impart desired pharmacokinetic properties and thus have an increased ability to cause the desired effect and thus prevent or treat the targeted diseases or conditions.

Compounds of formula (I) also comprise structural moieties that participate in inhibitory interactions with at least one subsite of beta-secretase. For example, moieties of the compounds of formula (I) may interact with at least one of the S1, S1′, and S2′ subsites, wherein S1 comprises residues Leu30, Tyr71, Phe108, Ile110, and Trp115, S1′ comprises residues Tyr198, Ile226, Val227, Ser 229, and Thr231, and S2′ comprises residues Ser₃₅, Asn37, Pro70, Tyr71, Ile118, and Arg128. Such compounds and methods of treatment may have an increased ability to cause the desired effect and thus prevent or treat the targeted diseases or conditions.

The term “pharmaceutically acceptable” refers to those properties and/or substances that are acceptable to the patient from a pharmacological/toxicological point of view, and to the manufacturing pharmaceutical chemist from a physical/chemical point of view regarding composition, formulation, stability, patient acceptance and bioavailability.

The term “effective amount” as used herein refers to an amount of a therapeutic agent administered to a host, as defined herein, necessary to achieve a desired effect.

The term “therapeutically effective amount” as used herein refers to an amount of a therapeutic agent administered to a host to treat or prevent a condition treatable by administration of a composition of the invention. That amount is the amount sufficient to reduce or lessen at least one symptom of the disease being treated or to reduce or delay onset of one or more clinical markers or symptoms of the disease.

The term “therapeutically active agent” refers to a compound or composition that is administered to a host, either alone or in combination with another therapeutically active agent, to treat or prevent a condition treatable by administration of a composition of the invention.

The term “pharmaceutically acceptable salt” and “salts thereof” refer to acid addition salts or base addition salts of the compounds in the present invention. A pharmaceutically acceptable salt is any salt which retains the activity of the parent compound and does not impart any deleterious or undesirable effect on the subject to whom it is administered and in the context in which it is administered. Pharmaceutically acceptable salts include salts of both inorganic and organic acids. Pharmaceutically acceptable salts include acid salts such as acetic, aspartic, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycolylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanilic, sulfonic, sulfuric, tannic, tartaric, teoclic, toluenesulfonic, and the like. Other acceptable salts may be found, for example, in Stahl et al., Pharmaceutical Salts: Properties, Selection, and Use, Wiley-VCH; 1st edition (Jun. 15, 2002).

In another embodiment of the present invention, a pharmaceutically acceptable salt is selected from hydrochloric, hydrobromic, hydroiodic, nitric, sulfuric, phosphoric, citric, methanesulfonic, CH₃—(CH₂)₀₋₄—COOH, HOOC—(CH₂)₀₋₄—COOH, HOOC—CH═CH—COOH, phenyl-COOH, and the like.

The term “unit dosage form” refers to physically discrete units suitable as unitary dosages for human subjects or other mammals, each unit containing a predetermined quantity of active material calculated to produce the desired therapeutic effect, in association with a suitable pharmaceutical vehicle. The concentration of active compound in the drug composition will depend on absorption, inactivation, and/or excretion rates of the active compound, the dosage schedule, the amount administered and medium and method of administration, as well as other factors known to those of skill in the art.

The term “modulate” refers to a chemical compound's activity to either enhance or inhibit a functional property of biological activity or process.

The terms “interact” and “interactions” refer to a chemical compound's association and/or reaction with another chemical compound, such as an interaction between an inhibitor and beta-secretase. Interactions include, but are not limited to, hydrophobic, hydrophilic, lipophilic, lipophobic, electrostatic, and van der Waal's interactions, and hydrogen bonding.

An “article of manufacture” as used herein refers to materials useful for the diagnosis, prevention or treatment of the disorders described above, such as a container with a label. The label can be associated with the article of manufacture in a variety of ways including, for example, the label may be on the container or the label may be in the container as a package insert. Suitable containers include, for example, blister packs, bottles, bags, vials, syringes, test tubes, and the like. The containers may be formed from a variety of materials such as glass, metal, plastic, rubber, paper, and-the like. The container holds a composition as described herein which is effective for diagnosing, preventing, or treating a condition treatable by a compound or composition of the present invention.

The article of manufacture may contain bulk quantities or less of a composition as described herein. The label on, or associated with, the container may provide instructions for the use of the composition in diagnosing, preventing, or treating the condition of choice, instructions for the dosage amount and for the methods of administration. The label may further indicate that the composition is to be used in combination with one or more therapeutically active agents wherein the therapeutically active agent is selected from an antioxidant, an anti-inflammatory, a gamma-secretase inhibitor, a neurotropic agent, an acetyl cholinesterase inhibitor, a statin, an A-beta, an anti-A-beta antibody, and/or a beta-secretase complex or fragment thereof. The article of manufacture may further comprise multiple containers, also referred to herein as a kit, comprising a therapeutically active agent or a pharmaceutically-acceptable buffer, such as phosphate-buffered saline, Ringer's solution and/or dextrose solution. It may further include other materials desirable from a commercial and user standpoint, including other buffers, diluents, filters, needles, syringes, and/or package inserts with instructions for use.

The compounds of formula (I), their compositions, and methods of treatment employing them, can be enclosed in multiple or single dose containers. The enclosed compounds and/or compositions can be provided in kits, optionally including component parts that can be assembled for use. For example, a compound of formula (I) in lyophilized form and a suitable diluent may be provided as separated components for combination prior to use. A kit may include a compound of formula (I) and at least one additional therapeutic agent for co-administration. The inhibitor (i.e., compound of formula (I)) and additional therapeutic agents may be provided as separate component parts.

A kit may include a plurality of containers, each container holding at least one unit dose of the compound of the present invention. The containers are preferably adapted for the desired mode of administration, including, for example, pill, tablet, capsule, powder, gel or gel capsule, sustained-release capsule, or elixir form, and/or combinations thereof and the like for oral administration, depot products, pre-filled syringes, ampoules, vials, and the like for parenteral administration, and patches, medipads, creams, and the like for topical administration.

The term “Cₘₐₓ” refers to the peak plasma concentration of a compound in a host.

The term “Tₘₐₓ” refers to the time at peak plasma concentration of a compound in a host.

The term “half-life” refers to the period of time required for the concentration or amount of a compound in a host to be reduced to exactly one-half of a given concentration or amount.

### DETAILED DESCRIPTION OF THE PRESENT INVENTION

The present invention is directed to novel compounds and methods of treating diseases, disorders, and conditions associated with amyloidosis. Amyloidosis refers to a collection of diseases, disorders, and conditions associated with abnormal deposition of amyloidal protein.

Accordingly, an aspect of the present invention is to provide a method of preventing or treating conditions associated with amyloidosis, comprising administering to a host thereof a composition comprising a therapeutically effective amount of at least one compound of formula (I), 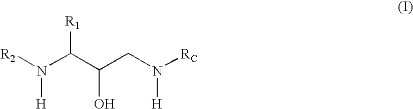
or pharmaceutically acceptable salts thereof, wherein R₁ is selected from 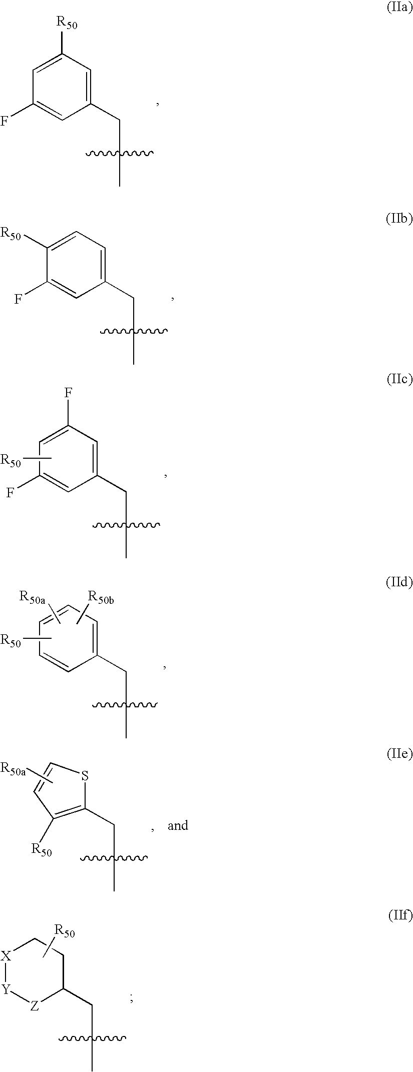
wherein X, Y, and Z are independently selected from —C(H)₀₋₂—, —O—, —C(O)—, —NH—, and —N—; wherein at least one bond of the (IIf) ring may optionally be a double bond; R₅₀, R₅₀ₐ, and R_{50b} are independently selected from —H, halogen, —OH, —SH, —CN, —C(O)-alkyl, —NR₇R₈, —S(O)₀₋₂-alkyl, alkyl, alkoxy, —O-benzyl (optionally substituted with at least one substituent independently selected from —H, —OH, and alkyl), —C(O)—NR₇R₈, alkyloxy, alkoxyalkoxyalkoxy, and cycloalkyl; wherein the alkyl, alkoxy, and cycloalkyl groups within R₅₀, R₅₀ₐ, and R_{50b} are optionally substituted with at least one substituent independently selected from alkyl, halogen, —OH, —NR₅R₆, —NR₇R₈, —CN, haloalkoxy, and alkoxy; R₅ and R₆ are independently selected from —H and alkyl; or R₅ and R₆, and the nitrogen to which they are attached, form a 5 or 6 membered heterocycloalkyl ring; and R₇ and R₈ are independently selected from —H, alkyl (optionally substituted with at least one group independently selected from —OH, —NH₂, and halogen), cycloalkyl, and -alkyl-O-alkyl;
R₂ is selected from —C(O)—CH₃, —C(O)—CH₂(halogen), —C(O)—CH(halogen)₂, 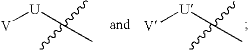
   U is selected from —C(O)—, —C(═S)—, —S(O)₀₋₂—, —C═N—R₂₁—, —C═N—OR₂₁—, —C(O)—NR₂₀—, —C(O)—O—, —S(O)₂—NR₂₀—, and —S(O)₂—O—; U′ is selected from —C(O)—, —C═N—R₂₁—, —C═N—OR₂₁—, —C(O)—NR₂₀—, and —C(O)—O—; V is selected from aryl, heteroaryl, cycloalkyl, heterocycloalkyl, —[C(R₄)(R₄)]₁₋₃-D, and -(T)₀₋₁, —R_{N}; V′ is selected from -(T)₀₋₁-R_{N′}; wherein the aryl, heteroaryl, cycloalkyl, and heterocycloalkyl groups included within V and V′ are optionally substituted with 1 or 2 R_{B} groups; wherein at least one carbon of the aryl, heteroaryl, cycloalkyl, and heterocycloalkyl groups included within V and V′ are optionally replaced with —N—, —O—, —NH—, —C(O)—, —C(S)—, —C(═N—H)—, —C(═N—OH)—, —C(═N-alkyl)-, or —C(═N—O-alkyl)-; R_{B} at each occurrence is independently selected from halogen, —OH, —CF₃, —OCF₃, —O-aryl, —CN, —NR₁₀₁R′₁₀₁, alkyl, alkoxy, —(CH₂)₀₋₄—(C(O))₀₋₁—(O)₀₋₁-alkyl, —C(O)—OH, —(CH₂)₀₋₃-cycloalkyl, aryl, heteroaryl, and heterocycloalkyl; wherein, the alkyl, alkoxy, cycloalkyl, aryl, heteroaryl, or heterocycloalkyl groups included within R_{B} are optionally substituted with 1 or 2 groups independently selected from —C₁-C₄ alkyl, —C₁-C₄ alkoxy, —C₁-C₄ haloalkyl, —C₁-C₄ haloalkoxy, -halogen, —OH, —CN, and —NR₁₀₁R′₁₀₁; R₁₀₁ and R′₁₀₁ are independently selected from —H, -alkyl, —(C(O))₀₋₁—(O)₀₋₁-alkyl, —C(O)—OH, and -aryl; R₄ and R₄ are independently selected from hydrogen, -alkyl, —(CH₂)₀₋₃-cycloalkyl, —(CH₂)₀₋₃—OH, -fluorine, —CF₃, —OCF₃, —O-aryl, -alkoxy, —C₃-C₇ cycloalkoxy, -aryl, and -heteroaryl, or R₄ and R₄ are taken together with the carbon to which they are attached to form a 3, 4, 5, 6, or 7 membered carbocyclic ring wherein 1, 2, or 3 carbons of the ring is optionally replaced with —O—, —N(H)—, —N(alkyl)-, —N(aryl)-, —C(O)—, or —S(O)₀₋₂; D is selected from aryl, heteroaryl, cycloalkyl, and heterocycloalkyl, wherein the aryl, heteroaryl, cycloalkyl, and heterocycloalkyl are optionally substituted with 1 or 2 R_{B} groups; T is selected from —NR₂₀— and —O—; R₂₀ is selected from H, —CN, -alkyl, -haloalkyl, and -cycloalkyl; R₂₁ is selected from —H, -alkyl, -haloalkyl, and -cycloalkyl; R_{N} is selected from —OH, —NH₂, —NH(alkyl), —NH(cycloalkyl), —N(alkyl)(alkyl), —N(alkyl)(cycloalkyl), —N(cycloalkyl)(cycloalkyl), —R₁₀₀, alkyl-R₁₀₀, —(CRR′)₁₋₆—P(O)(O-alkyl)₂, alkyl-O-alkyl-C(O)OH, —(CRR′)₁₋₆R′₁₀₀—(CRR′)₁₋₆R₁₀₀, —(CRR′)₁₋₆—O—R′₁₀₀, —(CRR′)₁₋₆—S—R′₁₀₀, —(CRR′)₁₋₆—C(O)—R₁₀₀, —(CRR′)₁₋₆—SO₂—R₁₀₀, and —(CRR′)₁₋₆—NR₁₀₀—R′₁₀₀ and —CH(R_{E1})—(CH₂)₀₋₃-E₁-E₂-E₃; R_{N′} is —SO₂R′₁₀₀; R and R′ are independently selected from -hydrogen, —C₁-C₁₀ alkyl (optionally substituted with at least one —OH), —C₁-C₁₀ alkylaryl, and —C₁-C₁₀ alkylheteroaryl; R₁₀₀ and R′₁₀₀ are independently selected from -cycloalkyl, -heterocycloalkyl, -aryl, -heteroaryl, alkoxy, -aryl-W-aryl, -aryl-W-heteroaryl, -aryl-W-heterocycloalkyl, -heteroaryl-W-aryl, -heteroaryl-W-heteroaryl, -heteroaryl-W-heterocycloalkyl, -heterocycloalkyl-W-aryl, -heterocycloalkyl-W-heteroaryl, -heterocycloalkyl-W-heterocycloalkyl, —W—R₁₀₂, —CH[(CH₂)₀₋₂—O—R₁₅₀]—(CH₂)₀₋₂-aryl, —CH[(CH₂)₀₋₂—O—R₁₅₀]—(CH₂)₀₋₂-cycloalkyl, —CH[(CH₂)₀₋₂—O—R₁₅₀]—(CH₂)₀₋₂-heterocycloalkyl, —CH[(CH₂)₀₋₂—O—R₁₅₀]—(CH₂)₀₋₂-heteroaryl, —C₁-C₁₀ alkyl (optionally substituted with 1, 2, or 3 R₁₁₅ groups), wherein 1, 2, or 3 carbons of the alkyl group are optionally replaced with a group independently selected from —C(O)— and —NH—, -alkyl-O-alkyl (optionally substituted with 1, 2, or 3 R₁₁₅ groups), -alkyl-5-alkyl (optionally substituted with 1, 2, or 3 R₁₁₅ groups), and -cycloalkyl (optionally substituted with 1, 2, or 3 R₁₁₅ groups); wherein the ring portions of each group included within R₁₀₀ and R′₁₀₀ are optionally substituted with 1, 2, or 3 groups independently selected from —OR, —NO₂, -halogen, —CN, —OCF₃, —CF₃, —(CH₂)₀₋₄—O—P(═O)(OR)(OR′), —(CH₂)₀₋₄—C(O)—NR₁₀₅R′₁₀₅, —(CH₂)₀₋₄—O—(CH₂)₀₋₄—C(O)NR₁₀₂R₁₀₂′, —(CH₂)₀₋₄—C(O)—(C₁-C₁₂ alkyl), —(CH₂)₀₋₄—C(O)—(CH₂)₀₋₄-cycloalkyl, —(CH₂)₀₋₄—R₁₁₀, —(CH₂)₀₋₄—R₁₂₀, —(CH₂)₀₋₄—R₁₃₀, —(CH₂)₀₋₄—C(O)—R₁₁₀, —(CH₂)₀₋₄—C(O)—R₁₂₀, —(CH₂)₀₋₄—C(O)—R_{13O}, —(CH₂)₀₋₄—C(O)—R₁₄₀, —(CH₂)₀₋₄—C(O)—O—R₁₅₀, —(CH₂)₀₋₄—SO₂—NR₁₀₅R′₁₀₅, —(CH₂)₀₋₄—SO—(C₁-C₈ alkyl), —(CH₂)₀₋₄—SO₂—(C₁-C₁₂ alkyl), —(CH₂)₀₋₄—SO₂—(CH₂)₀₋₄-cycloalkyl, —(CH₂)₀₋₄—N(R₁₅₀)—C(O)—O—R₁₅₀, —(CH₂)₀₋₄—N(R₁₅₀)—C(O)—N(R₁₅₀)₂, —(CH₂)₀₋₄—N(R₁₅₀)—CS—N(R₁₅₀)₂, —(CH₂)₀₋₄—N(R₁₅₀)—C(O)—R₁₀₅, —(CH₂)₀₋₄—NR₁₀₅R′₁₀₅, —(CH₂)₀₋₄—R₁₄₀, —(CH₂)₀₋₄—O—C(O)-(alkyl), —(CH₂)₀₋₄—C—P(O)—(O—R₁₁₀)₂, —(CH₂)₀₋₄—O—C(O)—N(R₁₅₀)₂—(CH₂)₀₋₄—O—CS—N(R₁₅₀)₂, —(CH₂)₀₋₄—O—(R₁₅₀), —(CH₂)₀₋₄—O—R₁₅₀′—C(O)OH, —(CH₂)₀₋₄—S—(R₁₅₀), —(CH₂)₀₋₄—N(R₁₅₀)—SO₂—R₁₀₅, —(CH₂)₀₋₄-cycloalkyl, and —(C₁-C₁₀)-alkyl; R_{E1} is selected from —H, —OH, —NH₂, —NH—(CH₂)₀₋₃—R_{E2}, —NHR_{E8}, —NR_{E35}OC(O)R_{E5}, —C₁-C₄ alkyl-NHC(O)R_{E5}, —(CH₂)₀₋₄R_{E8}, —O—(C₁-C₄ alkanoyl), —C₆-C₁₀ (aryloxy optionally substituted with 1, 2, or 3 groups that are independently selected from halogen, —C₁-C₄ alkyl, —CO₂H, —C(O)—C₁-C₄ alkoxy, and —C₁-C₄ alkoxy), alkoxy, -aryl-(C₁-C₄ alkoxy), —NR_{E350}CO₂R_{E351}, —C₁-C₄ alkyl-N R_{E350}CO₂R_{E351}, —CN, —CF₃, —CF₂—CF₃, —C≡CH, —CH₂—CH═CH₂, —(CH₂)₁₋₄—R_{E2}, —(CH₂)₁₋₄-NH-R_{E2}, —O—(CH₂)₀₋₃—R_{E2}, —S—(CH₂)₀₋₃—R_{E2}, —(CH₂)₀₋₄—NHC(O)—(CH₂)₀₋₆—R_{E352}, and —(CH₂)₀₋₄—(R_{E353})₀₋₁—(CH₂)₀₋₄—R_{E354}; R_{E2} is selected from —SO₂—(C₁-C₈ alkyl), —SO—(C₁-C₈ alkyl), —S—(C₁-C₈ alkyl), —S—C(O)-alkyl, —SO₂—NR_{E3}R_{E4}, —C(O)—C₁-C₂ alkyl, and —C(O)—NR_{E4}R_{E10}; R_{E3} and R_{E4} are independently selected from —H, —C₁-C₃ alkyl, and —C₃-C₆ cycloalkyl; R_{E10} is selected from alkyl, arylalkyl, alkanoyl, and arylalkanoyl; R_{E5} is selected from cycloalkyl, alkyl (optionally substituted with 1, 2, or 3 groups that are independently selected from halogen, —NR_{E6}R_{E7}, C₁-C₄ alkoxy, —C₅-C₆ heterocycloalkyl, —C₅-C₆ heteroaryl, —C₆-C₁₀ aryl, —C₃-C₇ cycloalkyl C₁-C₄ alkyl, —S—C₁-C₄ alkyl, —SO₂—C₁-C₄ alkyl, —CO₂H, —C(O)NR_{E6}R_{E7}, —CO₂—C₁-C₄ alkyl, and —C₆-C₁₀ aryloxy), heteroaryl (optionally substituted with 1, 2, or 3 groups that are independently selected from —C₁-C₄ alkyl, —C₁-C₄ alkoxy, halogen, —C₁-C₄ haloalkyl, and —OH), heterocycloalkyl (optionally substituted with 1, 2, or 3 groups independently selected from —C₁-C₄ alkyl, —C₁-C₄ alkoxy, halogen, and —C₂-C₄ alkanoyl), aryl (optionally substituted with 1, 2, 3, or 4 groups independently selected from halogen, —OH, —C₁-C₄ alkyl, —C₁-C₄ alkoxy, and —C₁-C₄ haloalkyl), and —NR_{E6}R_{E7}; R_{E6} and R_{E7} are independently selected from —H, alkyl, alkanoyl, aryl, —SO₂—C₁-C₄ alkyl, and -phenyl-C₁-C₄ alkyl; R_{E8} is selected from —SO₂-heteroaryl, —SO₂-aryl, —SO₂-heterocycloalkyl, —SO₂—C₁-C₁₀ alkyl, —C(O)NHR_{E9}, heterocycloalkyl, —S— alkyl, and —S—C₂-C₄ alkanoyl; R_{E9} is selected from H, alkyl, and -aryl C₁-C₄ alkyl; R_{E350} is selected from H and alkyl; R_{E351} is selected from alkyl, -aryl-(C₁-C₄ alkyl), alkyl (optionally substituted with 1, 2, or 3 groups independently selected from halogen, cyano, heteroaryl, —NR_{E6}R_{E7}, —C(O)NR_{E6}R_{E7}, —C₃-C₇ cycloalkyl, and —C₁-C₄ alkoxy), heterocycloalkyl (optionally substituted with 1 or 2 groups independently selected from —C₁-C₄ alkyl, —C₁-C₄ alkoxy, halogen, —C₂-C₄ alkanoyl, -aryl-(C₁-C₄ alkyl), and —SO₂—(C₁-C₄ alkyl)), heteroaryl (optionally substituted with 1, 2, or 3 groups independently selected from —OH, —C₁-C₄ alkyl, —C₁-C₄ alkoxy, halogen, —NH₂, —NH(alkyl), and —N(alkyl)(alkyl)), heteroarylalkyl (optionally substituted with 1, 2, or 3 groups independently selected from —C₁-C₄ alkyl, —C₁-C₄ alkoxy, halogen, —NH₂, —NH(alkyl), and —N(alkyl)(alkyl)), aryl, heterocycloalkyl, —C₃-C₈ cycloalkyl, and cycloalkylalkyl; wherein the aryl, heterocycloalkyl, —C₃-C₈ cycloalkyl, and cycloalkylalkyl groups included within R_{E351} are optionally substituted with 1, 2, 3, 4 or 5 groups independently selected from halogen, —CN, —NO₂, alkyl, alkoxy, alkanoyl, haloalkyl, haloalkoxy, hydroxy, hydroxyalkyl, alkoxyalkyl, —C₁-C₆ thioalkoxy, —C₁-C₆ thioalkoxy-alkyl, and alkoxyalkoxy; R_{E352} is selected from heterocycloalkyl, heteroaryl, aryl, cycloalkyl, —S(O)₀₋₂-alkyl, —CO₂H, —C(O)NH₂, —C(O)NH(alkyl), —C(O)N(alkyl)(alkyl), —CO₂-alkyl, —N HS(O)₀₋₂-alkyl, —N (alkyl)S(O)₀₋₂-alkyl, —S(O)₀₋₂-heteroaryl, —S(O)₀₋₂-aryl, —NH(arylalkyl), -N(alkyl)(arylalkyl), thioalkoxy, and alkoxy; wherein each group included within R₃₅₂ is optionally substituted with 1, 2, 3, 4, or 5 groups that are independently selected from alkyl, alkoxy, thioalkoxy, halogen, haloalkyl, haloalkoxy, alkanoyl, —NO₂, —CN, alkoxycarbonyl, and aminocarbonyl; R_{E353} is selected from —O—, —C(O)—, —NH—, —N(alkyl)-, —NH—S(O)₀₋₂—, —N(alkyl)-S(O)₀₋₂—, —S(O)₀₋₂—NH—, —S(O)₀₋₂—N(alkyl)-, —NH—C(S)—, and —N(alkyl)-C(S)—; R_{E354} is selected from heteroaryl, aryl, arylalkyl, heterocycloalkyl, —CO₂H, —CO₂-alkyl, —C(O)NH(alkyl), —C(O)N(alkyl)(alkyl), —C(O)NH₂, —C₁-C₈ alkyl, —OH, aryloxy, alkoxy, arylalkoxy, —NH₂, —NH(alkyl), —N(alkyl)(alkyl), and -alkyl-CO₂-alkyl; wherein each group included within R_{E354} is optionally substituted with 1, 2, 3, 4, or 5 groups that are independently selected from alkyl, alkoxy, —CO₂H, —CO₂-alkyl, thioalkoxy, halogen, haloalkyl, haloalkoxy, hydroxyalkyl, alkanoyl, —NO₂, —CN, alkoxycarbonyl, and aminocarbonyl; E₁ is selected from —NR_{E11}— and —C₁-C₆ alkyl-(optionally substituted with 1, 2, or 3 groups selected from —C₁-C₄ alkyl), and R_{E11} is selected from —H and alkyl; or R_{E1} and R_{E11} combine to form —(CH₂)₁₄—; E₂ is selected from a bond, —SO₂—, —SO—, —S—, and —C(O)—; and E₃ is selected from —H, —C₁-C₄ haloalkyl, —C₅-C₆ heterocycloalkyl, —C₆-C₁₀ aryl, —OH, —N(E₃ₐ)(E_{3b}), —C₁-C₁₀ alkyl (optionally substituted with 1, 2, or 3 groups independently selected from halogen, hydroxy, alkoxy, thioalkoxy, and haloalkoxy), —C₃-C₈ cycloalkyl (optionally substituted with 1, 2, or 3 groups independently selected from —C₁-C₃ alkyl and halogen), alkoxy, aryl (optionally substituted with at least one group selected from halogen, alkyl, alkoxy, —CN and —NO₂), arylalkyl (optionally substituted with a group selected from halogen, alkyl, alkoxy, —CN, and —NO₂); -E₃ₐ and E_{3b} are independently selected from —H, —C₁-C₁₀ alkyl (optionally substituted with 1, 2, or 3 groups independently selected from halogen, —C₁-C₄ alkoxy, —C₃-C₈ cycloalkyl, and —OH), —C₂-C₆ alkyl, —C₂-C₆ alkanoyl, aryl, —SO₂—C₁-C₄ alkyl, -aryl-C₁-C₄ alkyl, and —C₃-C₈ cycloalkyl C₁-C₄ alkyl; or -E₃ₐ, E_{3b}, and the nitrogen to which they are attached may optionally form a ring selected from piperazinyl, piperidinyl, morpholinyl, and pyrrolidinyl; wherein each ring is optionally substituted with 1, 2, 3, or 4 groups that are independently selected from alkyl, alkoxy, alkoxyalkyl, and halogen; W is selected from —(CH₂)₀₋₄—, —O—, —S(O)₀₋₂—, —N(R₁₃₅)—, —CR(OH)—, and —C(O)—; R₁₀₂ and R₁₀₂′ are independently selected from hydrogen, —OH, and —C₁-C₁₀ alkyl (optionally substituted with 1, 2, or 3 groups independently selected from -halogen, -aryl, and —R₁₁₀); R₁₀₅ and R′₁₀₅ are independently selected from —H, —R₁₁₀, —R₁₂₀, -cycloalkyl, —(C₁-C₂ alkyl)-cycloalkyl, -(alkyl)-O—(C₁-C₃ alkyl), and -alkyl (optionally independently substituted with at least one group selected from —OH, -amine, or -halogen); or R₁₀₅ and R′₁₀₅ together with the atom to which they are attached form a 3, 4, 5, 6, or 7 membered carbocyclic ring, wherein one member is optionally a heteroatom selected from —O—, —S(O)₀₋₂—, and —N(R₁₃₅)—, wherein the carbocyclic ring is optionally substituted with 1, 2 or 3 R₁₄₀ groups; and wherein at least one carbon of the carbocyclic ring is optionally replaced with —C(O)—; R₁₁₀ is aryl (optionally substituted with 1 or 2 R₁₂₅ groups); R₁₁₅ at each occurrence is independently selected from halogen, —OH, —C(O)—O—R₁₀₂, —C₁-C₆ thioalkoxy, —C(O)—O-aryl, —NR₁₀₅R′₁₀₅, —SO₂—(C₁-C₈ alkyl), —C(O)—R₁₈₀, R₁₈₀, —C(O)NR₁₀₅R′₁₀₅, —SO₂NR₁₀₅R′₁₀₅, —NH—C(O)-(alkyl), —NH—C(O)—OH, —NH—C(O)—OR, —NH—C(O)—O-aryl, —O—C(O)-(alkyl), —O—C(O)-amino, —O—C(O)-monoalkylamino, —O—C(O)-dialkylamino, —O—C(O)-aryl, —O-(alkyl)-C(O)—O—H, —NH—SO₂— (alkyl), -alkoxy, and -haloalkoxy; R₁₂₀ is -heteroaryl, (optionally substituted with 1 or 2 R₁₂₅ groups); R₁₂₅ at each occurrence is independently selected from -halogen, -amino, -monoalkylamino, -dialkylamino, —OH, —CN, —SO₂—NH₂, —SO₂—NH-alkyl, —SO₂—N(alkyl)₂, —SO₂—(C₁-C₄ alkyl), —C(O)—NH₂, —C(O)—NH-alkyl, —C(O)—N(alkyl)₂, -alkyl (optionally substituted with 1, 2, or 3 groups independently selected from C₁-C₃ alkyl, halogen, —OH, —SH, —CN, —CF₃, —C₁-C₃ alkoxy, -amino, -monoalkylamino, and -dialkylamino), and -alkoxy (optionally substituted with 1, 2, or 3-halogen); R₁₃₀ is heterocycloalkyl (optionally substituted with 1 or 2 R₁₂₅ groups; R₁₃₅ is independently selected from alkyl, cycloalkyl, —(CH₂)₀₋₂-(aryl), —(CH₂)₀₋₂-(heteroaryl), and —(CH₂)₀₋₂-(heterocycloalkyl); R₁₄₀ at each occurrence is independently selected from heterocycloalkyl (optionally substituted with 1, 2, 3, or 4 groups independently selected from -alkyl, -alkoxy, -halogen, -hydroxy, -cyano, -nitro, -amino, -monoalkylamino, -dialkylamino, -haloalkyl, -haloalkoxy, -amino-alkyl, -monoalkylamino-alkyl, and -dialkylaminoalkyl); and wherein at least one carbon of the heterocycloalkyl is optionally replaced with —C(O); R₁₅₀ is independently selected from -hydrogen, -cycloalkyl, —(C₁-C₂ alkyl)-cycloalkyl, —R₁₁₀, —R₁₂₀, and -alkyl (optionally substituted with 1, 2, 3, or 4 groups independently selected from —OH, —NH₂, —C₁-C₃ alkoxy, —R₁₁₀, and -halogen); R₁₅₀′ is independently selected from -cycloalkyl, —(C₁-C₃ alkyl)-cycloalkyl, —R₁₁₀, —R₁₂₀, and -alkyl (optionally substituted with 1, 2, 3, or 4 groups independently selected from —OH, —NH₂, —C₁-C₃ alkoxy, —R₁₁₀, and -halogen); and R₁₈₀ is independently selected from -morpholinyl, -thiomorpholinyl, -piperazinyl, -piperidinyl, -homomorpholinyl, -homothiomorpholinyl, -homothiomorpholinyl S-oxide, -homothiomorpholinyl S,S-dioxide, -pyrrolinyl, and -pyrrolidinyl; wherein each R₁₈₀ is optionally substituted with 1, 2, 3, or 4 groups independently selected from -alkyl, -alkoxy, -halogen, -hydroxy, -cyano, -nitro, -amino, -monoalkylamino, -dialkylamino, -haloalkyl, -haloalkoxy, -aminoalkyl, -monoalkylamino-alkyl, -dialkylamino-alkyl, and —C(O); and wherein at least one carbon of R₁₈₀ is optionally replaced with —C(O)—; R_{C} is 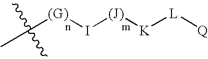
   n is 0 or 1; m is 0 or 1; G is selected from —C(O)— and —CO₂—; I is (CH₂)₀₋₄; J is selected from —(CR₂₄₅R₂₅₀)—; K is selected from aryl and heteroaryl; L is selected from a bond, -alkyl-(substituted with at least one group independently selected from R₂₀₅), —(CH₂)₀₋₄—(CO)₀₋₁—N(R₂₂₀)—, —(CH₂)₀₋₄—(CO)₀₋₁—, —(CH₂)₀₋₄—CO₂—, —(CH₂)₀₋₄—SO₂—N(R₂₂₀)—, —(CH₂)₀₋₄—N(H or R₂₁₅)—CO₂—, —(CH₂)₀₋₄—N(H or R₂₁₅)—SO₂—, —(CH₂)₀₋₄—N(H or R₂₁₅)—C(O)—N(R₂₁₅)—, —(CH₂)₀₋₄—N(H or R₂₁₅)—C(O)—, —(CH₂)₀₋₄—N(R₂₂₀)—, —(CH₂)₀₋₄—O—, and —(CH₂)₀₋₄—S—; Q is selected from aryl, heteroaryl, cycloalkyl, and heterocycloalkyl; wherein each cycloalkyl or heterocycloalkyl included within R_{C} is optionally substituted with at least one group independently selected from R₂₀₅; wherein each aryl or heteroaryl group included within R_{C} is optionally substituted with at least one group independently selected from R₂₀₀; wherein at least one heteroatom of the heteroaryl group included within R_{C} is optionally substituted with a group independently selected from —(CO)₀₋₁R₂₁₅, —(CO)₀₋₁R₂₂₀, and —S(O)₀₋₂R₂₀₀; R₂₀₀ at each occurrence is independently selected from alkyl (optionally substituted with at least one group independently selected from R₂₀₅), —OH, —NO₂, halogen, —CN, —(CH₂)₀₋₄—C(O)H, —(CO)₀₋₁R₂₁₅, —(CO)₀₋₁R₂₂₀, —(CH₂)₀₋₄—(CO)₀₋₁—NR₂₂₀R₂₂₅, —(CH₂)₀₋₄—C(O)-alkyl, —(CH₂)₀₋₄—(CO)O—-cycloalkyl, —(CH₂)₀₋₄—(CO)₀₋₁-heterocycloalkyl, —(CH₂)₀₋₄—(CO)₀₋₄-aryl, —(CH₂)₀₄—(CO)₀₁-heteroaryl, —(CH₂)₀₋₄—CO₂R₂₁₅, —(CH₂)₀₋₄—SO₂—NR₂₂₀R₂₂₅, —(CH₂)₀₋₄—S(O)₀₋₂-alkyl, —(CH₂)₀₋₄—S(O)₀₋₂-cycloalkyl, —(CH₂)₀₋₄—N(H or R₂₁₅)—CO₂R₂₁₅, —(CH₂)₀₋₄—N(H or R₂₁₅)—SO₂—R₂₂₀, —(CH₂)₀₋₄—N(H or R₂₁₅)—C(O)—N(R₂₁₅)₂, —(CH₂)₀₋₄—N(H or R₂₁₅)—C(O)—R₂₂₀, —(CH₂)₀₋₄—NR₂₂₀R₂₂₅, —(CH₂)₀₋₄—O—C(O)-alkyl, —(CH₂)₀₋₄—O—(R₂₁₅), —(CH₂)₀₋₄—S—(R₂₁₅), —(CH₂)₀₋₄—O-alkyl (optionally substituted with at least one halogen), and -adamantane; wherein each aryl and heteroaryl group included within R₂₀₀ is optionally substituted with at least one group independently selected from R₂₀₅, R₂₁₀, and alkyl (optionally substituted with at least one group independently selected from R₂₀₅ and R₂₁₀); wherein each cycloalkyl or heterocycloalkyl group included within R₂₀₀ is optionally substituted with at least one group independently selected from R₂₁₀; R₂₀₅ at each occurrence is independently selected-from -alkyl, -haloalkoxy, —(CH₂)₀₋₃-cycloalkyl, -halogen, —(CH₂)₀₋₆—OH, —O-aryl, —OH, —SH, —(CH₂)₀₋₄—C(O)H, —(CH₂)₀₋₆—CN, —(CH₂)₀₋₆—C(O)—NR₂₃₅R₂₄₀, —(CH₂)₀₋₆—C(O)—R₂₃₅, —(CH₂)₀₋₄—N(H or R₂₁₅)—SO₂—R₂₃₅, —CF₃, —CN, alkoxy, alkoxycarbonyl, and —NR₂₃₅R₂₄₀; R₂₁₀ at each occurrence is independently selected from —OH, —CN, —(CH₂)₀₋₄—C(O)H, alkyl (optionally substituted with at least one group independently selected from R₂₀₅), —S(O)₂-alkyl, halogen, alkoxy, haloalkoxy, —NR₂₂₀R₂₂₅, cycloalkyl (optionally substituted with at least one group independently selected from R₂₀₅), —C(O)-alkyl, —S(O)₂—NR₂₃₅R₂₄₀, —C(O)—NR₂₃₅R₂₄₀, and —S-alkyl; R₂₁₅ at each occurrence is independently selected from alkyl, —(CH₂)₀₋₂-cycloalkyl, —(CH₂)₀₋₂-aryl, —(CH₂)₀₋₂-heteroaryl, —(CH₂)₀₋₂-heterocycloalkyl, —CO₂—CH₂-aryl; wherein the aryl groups included within R₂₁₅ are optionally substituted with at least one group independently selected from R₂₀₅ and R₂₁₀, wherein the heterocycloalkyl and heteroaryl groups included within R₂₁₅ are optionally substituted with at least one group independently selected from R₂₁₀; R₂₂₀ and R₂₂₅ at each occurrence are independently selected from —H, alkyl, —(CH₂)₀₋₄—C(O)H, —(CH₂)₀₋₄—C(O)-alkyl, hydroxyalkyl, alkoxycarbonyl, alkylamino, —S(O)₂-alkyl, —C(O)-alkyl (optionally substituted with at least one halogen), —C(O)—NH₂, —C(O)—NH(alkyl), —C(O)—N(alkyl)(alkyl), haloalkyl, —(CH₂)₀₋₂cycloalkyl, -(alkyl)-O-(alkyl), aryl, heteroaryl, and heterocycloalkyl; wherein the aryl, heteroaryl and heterocycloalkyl groups included within R₂₂₀ and R₂₂₅ are each optionally substituted with at least one group independently selected from R₂₇₀; R₂₃₅ and R₂₄₀ at each occurrence are independently selected from —H, —OH, —CF₃, —OCH₃, —NH—CH₃, —N(CH₃)₂, —(CH₂)₀₋₄—C(O)—(H or alkyl), alkyl, —C(O)-alkyl, —SO₂-alkyl, and aryl; R₂₄₅ and R₂₅₀ at each occurrence are independently selected from —H, —OH, —(CH₂)₀₋₄CO₂-alkyl, —(CH₂)₀₋₄C(O)-alkyl, alkyl, hydroxyalkyl, alkoxy, haloalkoxy, —(CH₂)₀₋₄-cycloalkyl, —(CH₂)₀₋₄-aryl, —(CH₂)₀₋₄-heteroaryl, and —(CH₂)₀₋₄-heterocycloalkyl; or R₂₄₅ and R₂₅₀ are taken together with the carbon to which they are attached to form a monocyclic or bicyclic ring system of 3, 4, 5, 6, 7, or 8 carbon atoms; wherein at least one carbon atom is optionally replaced by at least one group independently selected from —O—, —S—, —SO₂—, —C(O)—, —NR₂₂₀—, and —N(alkyl)(alkyl); and wherein the ring is optionally substituted with at least one group independently selected from alkyl, alkoxy, —OH, —NH₂, —NH(alkyl), —N(alkyl)(alkyl), —NH—C(O)-alkyl, —NH—SO₂-alkyl, and halogen; wherein the aryl, heteroaryl, and heterocycloalkyl groups included within R₂₄₅ and R₂₅₀ are optionally substituted with at least one group independently selected from halogen, alkyl, —CN, and —OH; R₂₇₀ at each occurrence is independently selected from —R₂₀₅, alkyl (optionally substituted with at least one group independently selected from R₂₀₅), aryl, halogen, alkoxy, haloalkoxy, —NR₂₃₅R₂₄₀, —OH, —CN, cycloalkyl (optionally substituted with at least one group independently selected from R₂₀₅), —C(O)-alkyl, —S(O)₂—NR₂₃₅R₂₄₀, —CO—NR₂₃₅R₂₄₀, —S(O)₂-alkyl, and —(CH₂)₀₋₄—C(O)H.

An embodiment of the present invention is to provide selective compounds of formula (I), or pharmaceutically acceptable salts thereof, wherein R₁, R₂, and R_{C} are defined above.

Another embodiment of the present invention is to provide efficacious compounds of formula (I), or pharmaceutically acceptable salts thereof, wherein the inhibition is at least 10% for a dose of about 100 mg/kg or less, and wherein R₁, R₂, and R_{C} are defined above.

Another embodiment of the present invention is to provide orally bioavailable compounds of formula (I), or pharmaceutically acceptable salts thereof, wherein said compound has an F value of at least 10%, and wherein R₁, R₂, and R_{C} are defined above.

In an embodiment, the present invention provides a method of preventing or treating conditions which benefit from inhibition of at least one aspartyl-protease, comprising administering to a host in need thereof a composition comprising a therapeutically effective amount of at least one compound of the formula, 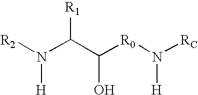
or pharmaceutically acceptable salts thereof, wherein R₁, R₂, and R_{C} are as defined below and R₀ is selected from —CH(alkyl), —C(alky)₂—, —CH(cycloalkyl)-, —C(alkyl)(cycloalkyl)-, and —C(cycloalkyl)₂.

In an embodiment, the hydroxyl alpha to the —(CHR₁)— group of formula (I) may be optionally replaced by —NH₂, —NHR₇₀₀, —NR₇₀₀R₇₀₀, —SH, and —SR₇₀₀, wherein R₇₀₀ is alkyl (optionally substituted with at least one group independently selected from R₁₁₀, R₁₁₅, R₂₀₅, and R₂₁₀); wherein R₁₁₀, R₁₁₅, R₂₀₅, and R₂₁₀ are defined herein.

In another embodiment, R₁ is selected from —CH₂-phenyl, wherein the phenyl ring is optionally substituted with at least one group independently selected from halogen, alkyl, alkoxy, and —OH.

In another embodiment, R₁ is selected from 3-Allyloxy-5-fluoro-benzyl, 3-Benzyloxy-5-fluoro-benzyl, 4-hydroxy-benzyl, 3-hydroxy-benzyl, 3-propyl-thiophen-2-yl-methyl, 3,5-difluoro-2-propylamino-benzyl, 5-chloro-thiophen-2-yl-methyl, 5-chloro-3-ethyl-thiophen-2-yl-methyl, 3,5-difluoro-2-hydroxy-benzyl, 2-ethylamino-3,5-difluoro-benzyl, piperidin-4-yl-methyl, 2-oxo-piperidin-4-yl-methyl, 2-oxo-1,2-dihydro-pyridin-4-yl-methyl, 5-hydroxy-6-oxo-6H-pyran-2-yl-methyl, 2-Hydroxy-5-methyl-benzamide, 3,5-Difluoro-4-hydroxy-benzyl, 3,5-Difluoro-benzyl, 3-Fluoro-4-hydroxy-benzyl, 3-Fluoro-5-[2-(2-methoxy-ethoxy)-ethoxy]-benzyl, 3-Fluoro-5-heptyloxy-benzyl, 3-Fluoro-5-hexyloxy-benzyl, 3-Fluoro-5-hydroxy-benzyl, and 3-Fluoro-benzyl.

In another embodiment, R₂ is selected from —C(O)—CH₃ and —C(O)—CH₂F.

In another embodiment, R₂ is selected from tert-butyl formate, 2,2-difluoroacetaldehyde, 2-hydroxyacetaldehyde, hydrosulfonylmethane, N-(3-formylphenyl)methanesulfonamide, and N-(3-formylphenyl)-N-methylmethanesulfonamide,

In another embodiment, R₂ is selected from glyoxylic acid, crotonic acid, pyruvic acid, butyric acid, sarcosine, 3-hydroxy-propionic acid, methoxyacetic acid, chloroacetic acid, penta-2,4-dienoic acid, pent-4-ynoic acid, 1-methyl-cyclopropanecarboxylic acid, pent-4-enoic acid, cyclopropylacetic acid, cyclobutanecarboxylic acid, trans-2-pentenoic acid, valeric acid, DL-2-ethylpropionic acid, isovaleric acid, 2-hydroxy-2-methyl-propionic acid, ethoxyacetic acid, DL-2-hydroxy-n-butyric acid, furan-3-carboxylic acid, 1H-pyrazole-4-carboxylic acid, 1H-imidazole-4-carboxylic acid, cyclopent-1-enecarboxylic acid, 4-Methyl-pent-2-enoic acid, cyclopentanecarboxylic acid, trans-2-hexenoic acid, 2-oxo-pentanoic acid, levulinic acid, tetrahydro-3-fluroic acid, tetrahydrofuran-2-carboxylic acid, caproic acid, tert-butylacetic acid, methylmalonic acid, 2-hydroxy-3-methyl-butyric acid, benzoic acid, 2-chloro-butyric acid, picolonic acid, nicotinic acid, isonicotinic acid, pyrazine-2-carboxylic acid, 3-methyl-furan-2-carboxylic acid, 1-methyl-1H-pyrazole-3-carboxylic acid, cyclopent-2-enyl-acetic acid, 5-methyl-isoxazole-3-carboxylic acid, thiophene-3-carboxylic acid, 2-Methyl-hex-2-enoic acid, L-pyroglutamic acid, 5-oxo-pyrrolidine-2-carboxylic acid, D-pyroglutamic acid, N-methylaleamic acid, thiazole 5-carboxylic acid, N-Me-Pro —OH, 3-Methyl-pyrrolidine-2-carboxylic acid, itaconic acid, citraconic acid, 2-oxo-imidazolidine-4-carboxylic acid, 4-Methyl-2-oxo-pentanoic acid, enanthic acid, L-hydroxyproline, Cis-4-hydroxy-D-proline, 6-Amino-hexanoic acid, oxalacetic acid, Mono-methyl succinate, Butoxy-acetic acid, (S)-(−)-2-hydroxy-3,3-dimethylbutyric acid, (2-methoxy-ethoxy)-acetic acid, Phenylacetic acid, 5-Chloro-pentanoic acid, Anthranilic acid, Aminonicotinic acid, 3-Hydroxy-pyridine-2-carboxylic acid, 2-Hydroxy-nicotinic acid, Furan-2-yl-oxo-acetic acid, 5-Formyl-furan-2-carboxylic acid, 6-Hydroxy-pyrimidine-4-carboxylic acid, 3-Furan-2-yl-propionic acid, Norbornane-2-carboxylic acid, 1-cyclohexenylacetic acid, 3,5-Dimethyl-isoxazole-4-carboxylic acid, Hexa-2,4-dienedioic acid, (2-Oxo-cyclopentyl)-acetic acid, 5-Methyl-thiophene-2-carboxylic acid, Thiophene-2-acetic acid, cylcohexylacetic acid, methyl cyclohexanone-2-carboxylate, (2-Imino-imidazolidin-1-yl)-acetic acid, 4-amino-cyclohexanecarboxylic acid, 2-methylene-succinic acid 1-methyl ester, Trans-beta-hydromuconic acid, Octanoic acid, 2-Propyl-pentanoic acid, 4-Acetylamino-butyric acid, 2-Oxo-pentanedioic acid, N-carbamyl-alpha-aminoisobutyric acid, 4-cyano-benzoic acid, and 2-Acetylamino-3-hydroxy-propionic acid.

In another embodiment, U is selected from —C(O)—, —C(S)—, —S(O)₀₋₂—, —C(NR₂₁)—, —C(N—OR₂₁)—, —C(O)—NR₂₀—, —C(O)—O—, —S(O)₂—NR₂₀—, and —S(O)₂—O—; and V is -(T)₀₋₁—R_{N}.

In another embodiment, U is —C(O)—.

In another embodiment, U is selected from —C(O)— and —S(O)₀₋₂—; and V is selected from alkyl, alkoxy, aryl, heteroaryl, cycloalkyl, and heterocycloalkyl; wherein the alkyl included within V are optionally substituted with at least one group independently selected from —OH, —NH₂, and halogen; and wherein the aryl, heteroaryl, cycloalkyl, and heterocycloalkyl groups included within V are optionally substituted with 1 or 2 R_{B} groups.

In another embodiment, U′ is selected from —C(O)—, —C(NR₂₁)—, —C(N—OR₂₁)—, —C(O)—NR₂₀—, and —C(O)—O—; and V′is -(T)₀₋₁-R_{N′}.

In another embodiment, R_{N} is selected from alkyl, —(CH₂)₀₋₂-aryl, C₂-C₆ alkyl, C₃-C₇ cycloalkyl, —(CH₂)₀₋₂-heteroaryl, and 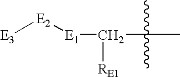
wherein E₁ is selected from —NR_{E11}— and C₁-C₆ alkyl optionally substituted with 1, 2, or 3 C₁-C₄ groups, R_{E1} is —NH₂, and R_{E11} is selected from —H and alkyl, or R_{E1} and R_{E11} combine to form —(CH₂)₁₋₄—; E₂ is selected from a bond; SO₂, SO, S, and C(O); E₃ is selected from —H, —C₁-C₄ haloalkyl, —C₅-C₆ heterocycloalkyl containing at least one N, O, or S, -aryl, —OH, —N(E₃ₐ)(E_{3b}), —C₁-C₁₀ alkyl optionally substituted with 1, 2, or thru 3 groups which can be the same independently or different and are se selected from halogen, hydroxy, alkoxy, thioalkoxy, and haloalkoxy, —C₃-C₈ cycloalkyl optionally substituted with 1, 2, or 3 groups independently selected from C₁-C₃ alkyl, and halogen, -alkoxy, -aryl optionally substituted with at least one group selected from halogen, C₁-C₄ alkyl, C₁-C₄ alkoxy, —CN, and —NO₂ and -aryl C₁-C₄ alkyl optionally substituted with at least one group selected from halogen, C₁-C₄ alkyl, C₁-C₄ alkoxy, —CN, and —NO₂, E₃ₐ and E_{3b} are independently selected from —H, —C₁-C₁₀ alkyl optionally substituted with 1, 2, or 3 groups independently selected from halogen, C₁-C₄ alkoxy, C₃-C₈ cycloalkyl, and —OH, —C₂-C₆ alkanoyl, -aryl, —SO₂—C₁-C₄ alkyl, -aryl C₁-C₄ alkyl, and —C₃-C₈ cycloalkyl C₁-C₄ alkyl, or E₃ₐ, E_{3b}, and the nitrogen to which they are attached form a ring selected from piperazinyl, piperidinyl, morpholinyl, and pyrolidinyl, wherein each ring is optionally substituted with 1, 2, 3, or 4 groups that are independently selected from alkyl, alkoxy, alkoxyalkyl, and halogen.

In another embodiment, V is —(CH₂)₁₋₃-aryl or —(CH₂)₁₋₃-heteroaryl, wherein each ring is independently optionally substituted with 1 or 2 groups independently selected from halogen, —OH, —OCF₃, —O-aryl, —CN, —NR₁₀₁R′₁₀₁, alkyl, alkoxy, (CH₂)₀₋₃(C₃-C₇ cycloalkyl), aryl, heteroaryl, and heterocycloalkyl, and wherein the alkyl, alkoxy, cycloalkyl, aryl, heteroaryl, or heterocycloalkyl groups are optionally substituted with 1 or 2 groups independently selected from C₁-C₄ alkyl, C₁-C₄ alkoxy, C₁-C₄ haloalkyl, C₁-C₄ haloalkoxy, halogen, —OH, —CN, and —NR₁₀₁R′₁₀₁.

In another embodiment, R_{C} is selected from 5-(2,2-dimethyl-propyl)-2-(2-propyl-imidazol-1-yl)-benzyl, 5-(2,2-dimethyl-propyl)-2-(1H-pyrrol-2-yl)-benzyl, 5-(2,2-dimethyl-propyl)-2-(imidazol-1-yl)-benzyl, 5-(2,2-dimethyl-propyl)-2-(1H-pyrazol-4-yl)-benzyl, 5(2,2-dimethyl-propyl)-2-[1,2,3]thiadiazol-4-yl-benzyl, 5-(2,2-dimethyl-propyl)-2-thiazol-5-yl-benzyl, 5-(2,2-dimethyl-propyl)-2-(3-methyl-isothiazol-5-yl)-benzyl, 5-(2,2-dimethyl-propyl)-2-(2H-[1,2,3] triazol-4-yl)-benzyl, 5-(2,2-dimethyl-propyl)-2-pyridin-3-yl-benzyl, 5-(2,2-dimethyl-propyl)-2-(6-fluoro-pyridin-3-yl)-benzyl, 5-(2,2-dimethyl-propyl)-2-(2-fluoro-pyridin-3-yl)-benzyl, 5-(2,2-dimethyl-propyl)-2-pyridazin-3-yl-benzyl, 5-(2,2-dimethyl-propyl)-2-pyrimidin-5-yl-benzyl, 5-(2,2-dimethyl-propyl)-2-imidazol-1-yl-phenyl, -cyclopropyl, 5-(2,2-dimethyl-propyl)-2-pyrazin-2-yl-benzyl, 5-(2,2-dimethyl-propyl)-2-(5-ethyl-imidazol-1-yl)-benzyl, 3-Chloro-5-(2,2-dimethyl-propyl)-2-imidazol-1-yl-benzyl, 5-(2,2-dimethyl-propyl)-2-tetrazol-1-yl-benzyl, and 5-(2,2-dimethyl-propyl)-2-imidazol-1-yl-benzyl.

An embodiment of the present invention is compounds of formula (I), or pharmaceutically acceptable salts thereof, wherein R and R′ are independently selected from hydrogen and —C₁-C₁₀ alkyl (substituted with at least one group selected from OH).

In another embodiment, R_{B} is selected from —CF₃, —C(O)₀₋₁—(O)₀₋₁-alkyl, —C(O)₀₋₁—OH.

In another embodiment, R_{N} is selected alkyl-R₁₀₀, —NH₂, —OH, —(CRR′)₁₋₆—P(O)(O-alkyl)₂, and alkyl-O-alkyl-C(O)OH.

In another embodiment, R₄ and R_{4′} are independently selected from —OH.

In another embodiment, R₁₀₀ and R′₁₀₀ are independently selected from alkoxy.

In another embodiment, R₁₀₁ and R′₁₀₁ are independently selected from —C(O)₀₋₁—(O)₀₋₁-alkyl and —C(O)₀₋₁—OH.

In another embodiment, R₁₁₅ is —NH—C(O)-(alkyl).

In another embodiment, R₂₀₀ is —(CH₂)₀₋₄—C(O)—NH(R₂₁₅).

In another embodiment, R₂₀₅ is selected from —(CH₂)₀₋₆—C(O)—R₂₃₅, —(CH₂)₀₋₄—N(H or R₂₁₅)—SO₂—R₂₃₅, —CN, and —OCF₃.

In another embodiment, R₂₁₀ is selected from heterocycloalkyl, heteroaryl, —(CO)₀₋₁R₂₁₅, —(CO)₀₋₁R₂₂₀, —(CH₂)₀₋₄—NR₂₃₅R₂₄₀, —(CH₂)₀₋₄—N R₂₃₅(alkoxy), —(CH₂)₀₋₄—S—(R₂₁₅), —(CH₂)₀₋₆—OH, —(CH₂)₀₋₆—CN, —(CH₂)₀₋₄—NR₂₃₅—C(O)H, —(CH₂)₀₋₄—NR₂₃₅—C(O)-(alkoxy), —(CH₂)₀₋₄—NR₂₃₅—C(O)—R₂₄₀, and —C(O)—NHR₂₁₅.

In another embodiment, R₂₃₅ and R₂₄₀ are independently selected from —OH, —CF₃, —OCH₃, —NH—CH₃, —N(CH₃)₂, —(CH₂)₀₋₄—C(O)—(H or alkyl).

In another embodiment, D is cycloalkyl.

In another embodiment, E₁ is C₁-C₄ alkyl.

In another embodiment, V is cycloalkyl.

In another embodiment, at least one carbon of the aryl, heteroaryl, cycloalkyl, and heterocycloalkyl groups included within V and V′ are optionally replaced with a group selected form —C(O)—, —C(S)—, —C(═N—H)—, —C(═N—OH)—, —C(═N-alkyl)—, and —C(═N—O-alkyl)—, —C(O)₀₋₁—(O)₀₋₁-alkyl, and C(O)₀₋₁—OH.

Among the compounds of formula (I), examples include N-{1-(3,5-difluoro-benzyl)-3-[5-(2,2-dimethyl-propyl)-2-(2-propyl-imidazol-1-yl)-benzylamino]-2-hydroxy-propyl}-acetamide, N-(1-(3,5-difluorophenyl)-3-hydroxy-4-(5-neopentyl-2-(1H-pyrrol-2-yl)benzylamino)butan-2-yl)acetamide, N-(4-(2-(1H-imidazol-1-yl)-5-neopentylbenzylamino)-1-(3,5-difluorophenyl)-3-hydroxybutan-2-yl)acetamide, N-(4-(2-(1H-imidazol-1-yl)-5-neopentylbenzylamino)-1-(3,5-difluorophenyl)-3-hydroxybutan-2-yl)acetamide, N-{1-(3,5-difluoro-benzyl)-3-[5-(2,2-dimethyl-propyl)-2-(1H-pyrazol-4-yl)-benzylamino]-2-hydroxy-propyl}-acetamide, N-{1-(3,5-difluoro-benzyl)-3-[5-(2 ,2dimethyl-propyl)-2-[1,2,3]thiadiazol-4-yl-benzylamino]-2-hydroxy-propyl}-acetamide, N-{1-(3,5-difluoro-benzyl)-3-[5-(2,2-dimethyl-propyl)-2-thiazol-5-yl-benzylamino]-2-hydroxy-propyl}-acetamide, N-{1-(3,5-difluoro-benzyl)-3-[5-(2,2-dimethyl-propyl)-2-(3-methyl-isothiazol-5-yl)-benzylamino]-2-hydroxy-propyl}-acetamide, N-{1-(3,5-difluoro-benzyl)-3-[5-(2,2-dimethyl-propyl)-2-(2H-[1,2,3]triazol-4-yl)-benzylamino]-2-hydroxy-propyl}-acetamide, N-(1-(3,5-difluorophenyl)-3-hydroxy-4-(5-neopentyl-2-(pyridin-3-yl)benzylamino)butan-2-yl)acetamide, N-(1-(3,5-difluorophenyl)-4-(2-(6-fluoropyridin-3-yl)-5-neopentylbenzylamino)-3-hydroxybutan-2-yl)acetamide, N-(4-(2-(3-acetylthiophen-2-yl)-5-neopentylbenzylamino)-1-(3,5-difluorophenyl)-3-hydroxybutan-2-yl)acetamide, N-{1-(3,5-difluoro-benzyl)-3-[5-(2,2-dimethyl-propyl)-2-(2-fluoro-pyridin-3-yl)-benzylamino]-2-hydroxy-propyl}-acetamide, N-{1-(3,5-difluoro-benzyl)-3-[5-(2,2-dimethyl-propyl)-2-pyridazin-3-yl-benzylamino]-2-hydroxy-propyl}-acetamide, N-{1-(3,5-difluoro-benzyl)-3-[5-(2,2-dimethyl-propyl)-2-pyrimidin-5-yl-benzylamino]-2-hydroxy-propyl}-acetamide, N-(1-(3,5-difluoro-benzyl)-3-{1-[5-(2,2-dimethyl-propyl)-2-imidazol-1-yl-phenyl-cyclopropylamino}-2-hydroxy-propyl}-acetamide, N-{1-(3,5-difluoro-benzyl)-3-[5-(2,2-dimethyl-propyl)-2-pyrazin-2-yl-benzylamino]-2-hydroxy-propyl-acetamide, N-{1-(3,5-difluoro-benzyl)-3-[5-(2,2-dimethyl-propyl)-2-(5-ethyl-imidazol-1-yl)-benzylamino]-2-hydroxy-propyl}-acetamide, N-[3-[3-chloro-5-(2,2-dimethyl-propyl)-2-imidazol-1-yl-benzylamino]-1-(3,5-difluoro-benzyl)-2-hydroxy-propyl]-acetamide, N-{1-(3,5-difluoro-benzyl)-3-[5-(2,2-dimethyl-propyl)-2-tetrazol-1-yl-benzylamino]-2-hydroxy-propyl}-acetamide, N-(1-(3,5-difluorophenyl)-4-(2-(3,5-dimethylisoxazol-4-yl)-5-neopentylbenzylamino)-3-hydroxybutan-2-yl)acetamide, N-{1-(3,5-Difluoro-benzyl)-2-hydroxy-3-[1-(3-thiazol-2-yl-phenyl)-cyclopropylamino]-propyl}-acetamide, N-{1-(3,5Difluoro-benzyl)-3-[2-(2,2-dimethyl-propyl)-5-(4-hydroxymethyl-imidazol-1-yl)-benzylamino]-2-hydroxy-propyl}-acetamide, N-{1-(3,5-Difluoro-benzyl)-3-[2-(2,2-dimethyl-propyl)-5-thiophen-2-yl-benzylamino]-2-hydroxy-propyl}-acetamide, N-[3-[5-(3-Acetyl-thiophen-2-yl)-2-(2,2-dimethyl-propyl)-benzylamino]-1-(3,5-difluoro-benzyl)-2-hydroxy-propyl]-acetamide, N-{1-(3,5-Difluoro-benzyl)-3-[2-(2,2-dimethyl-propyl)-5-furan-3-yl-benzylamino]-2-hydroxy-propyl}-acetamide, N-{1-(3,5-Difluoro-benzyl)-3-[2-(2,2-dimethyl-propyl)-5-furan-2-yl-benzylamino]-2-hydroxy-propyl)-acetamide, N-{1-(3,5-Difluoro-benzyl)-0.3-[2-(2,2-dimethyl-propyl)-5-(1H-pyrrol-2-yl)-benzylamino]-2-hydroxy-propyl}-acetamide, N-{1-(3,5-Difluoro-benzyl)-3-[2-(2,2-dimethyl-propyl)-5-(4-methyl-thiophen-2-yl)-benzylamino]-2-hydroxy-propyl}-acetamide, N-{1-(3,5-Difluoro-benzyl)-3-[2-(2,2-dimethyl-propyl)-5-thiophen-3-yl-benzylamino]-2-hydroxy-propyl}-acetamide, N-[3-[5-Benzofuran-2-yl-2-(2,2-dimethyl-propyl)-benzylamino]-1-(3,5-difluoro-benzyl)-2-hydroxy-propyl]-acetamide, N-[3-[5-Benzo[b]thiophen-2-yl-2-(2,2-dimethyl-propyl)-benzylamino]-1-(3,5-difluoro-benzyl)-2-hydroxy-propyl]-acetamide, N-{1-(3,5-Difluoro-benzyl)-3-[2-(2,2-dimethyl-propyl)-5-(1-propyl-1H-pyrazol-4-yl)-benzylamino]-2-hydroxy-propyl}-acetamide, N-{1-(3,5-Difluoro-benzyl)-3-[2-(2,2-dimethyl-propyl)-5-(2-formyl-thiophen-3-yl)-benzylamino]-2-hydroxy-propyl}-acetamide, N-{1-(3,5-Difluoro-benzyl)-3-[2-(2,2-dimethyl-propyl)-5-(5-formyl-thiophen-2-yl)-benzylamino]-2-hydroxy-propyl}-acetamide, N-(1-(3,5-difluorophenyl)-3-hydroxy-4-(1-(2-(thiazol-2-yl)phenyl)cyclopropylamino)butan-2-yl)acetamide, N-(1-(3,5-difluorophenyl)-3-hydroxy-4-(5-neopentyl-2-(thiophen-2-yl)benzylamino)butan-2-yl)acetamide, N-(4-(2-(5-acetylthiophen-2-yl)-5-neopentylbenzylamino)-1-(3,5-difluorophenyl)-3-hydroxybutan-2-yl)acetamide, N-(1-(3,5-difluorophenyl)-4-(2-(fu ran-3-yl)-5-neopentylbenzylamino)-3hydroxybutan-2-yl)acetamide, N-(1-(3,5-difluorophenyl)-4-(2-(furan-2-yl)-5-neopentylbenzylamino)-3-hydroxybutan-2-yl)acetamide, N-(1-(3,5-difluorophenyl)-3-hydroxy-4-(5-neopentyl-2-(thiophen-3-yl)benzylamino)butan-2-yl)acetamide, N-(1-(3,5-difluorophenyl)-3-hydroxy-4-(2-(4-methylthiophen-2-yl)-5-neopentylbenzylamino)butan-2-yl)acetamide, N-(4-(2-(benzofuran-2-yl)-5-neopentylbenzylamino)-1-(3,5-difluorophenyl)-3-hydroxybutan-2-yl)acetamide, N-(1-(3,5-difluorophenyl)-3-hydroxy-4-(5-neopentyl-2-(1-propyl-1H-pyrazol-4-yl)benzylamino)butan-2-yl)acetamide, N-(4-(2-(1H-indol-2-yl)-5-neopentylbenzylamino)-1-(3,5-difluorophenyl)-3-hydroxybutan-2-yl)acetamide, N-(1-(3,5-difluorophenyl)-3-hydroxy-4-(2-(1-methyl-1H-pyrazol-4-yl)-5-neopentylbenzylamino)butan-2-yl)acetamide, N-(1-(3,5-difluorophenyl)-3-hydroxy-4-(5-neopentyl-2-(1H-pyrazol-4-yl)benzylamino)butan-2-yl)acetamide, N-(1-(3,5-difluorophenyl)-3-hydroxy-4-(2-(5-methylthiophen-2-y)-5-neopentylbenzylamino)butan-2-yl)acetamide, N-(1-(3,5-difluorophenyl)-4-(2-(2-formylthiophen-3-yl)-5-neopentylbenzylamino)-3-hydroxybutan-2-yl)acetamide, N-(1-(3,5-difluorophenyl)-4-(2-(5-formylthiophen-2-yl)-5-neopentylbenzylamino)-3-hydroxybutan-2-yl)acetamide, N-(4-(2-(benzo[b]thiophen-2-yl)-5-neopentylbenzylamino)-1-(3,5-difluorophenyl)-3-hydroxybutan-2-yl)acetamide, N-{1-(3,5-difluoro-benzyl)-3-[5-(2,2-dimethyl-propyl)-2-imidazol-1-yl-benzylamino]-2-hydroxy-propyl}-acetamide, N-(1-(3,5-difluorophenyl)-3-hydroxy-4-(2-(4-methyl-1H-imidazol-1-yl)-5-neopentylbenzylamino)butan-2-yl)acetamide, N-(1-(3,5-difluorophenyl)-3-hydroxy-4-(5-neopentyl-2-(4-phenyl-1H-imidazol-1-yl)benzylamino)butan-2-yl)acetamide, N-(4-(2-(1H-benzo[d]imidazol-1-yl)-5-neopentylbenzylamino)-1-(3,5-difluorophenyl)-3-hydroxybutan-2-yl)acetamide, N-(4-(2-(3-acetyl-1H-pyrrol-1-yl)-5-neopentylbenzylamino)-1-(3,5-difluorophenyl)-3hydroxybutan-2-yl)acetamide, 1-(2-((3-acetamido-4-(3,5-difluorophenyl)-2-hydroxybutylamino)methyl)-4-neopentylphenyl)-1H-imidazole-4-carboxylic acid, N-{1-(3,5-Difluoro-benzyl)-3-[5-(2,2-dimethyl-propyl)-2-indol-1-yl-benzylamino]-2-hydroxy-propyl}-acetamide, N-(4-(2-(1H-indol-1-yl)-5-neopentylbenzylamino)-1-(3,5-difluorophenyl)-3-hydroxybutan-2-yl)acetamide, N-(1-(3,5-difluorophenyl)-3-hydroxy-4-(5-neopentyl-2-(1H-pyrazol-1-yl)benzylamino)butan-2-yl)acetamide, N-(4-(2-(3-acetyl-1H-pyrazol-1-yl)-5-neopentylbenzylamino)-1-(3,5-difluorophenyl)-3-hydroxybutan-2-yl)acetamide, N-(1-(3,5-difluorophenyl)-3-hydroxy-4-(2-(3-methyl-1H-pyrazol-1-yl)-5-neopentylbenzylamino)butan-2-yl)acetamide, N-{1-(3,5-Difluoro-benzyl)-3-[5-(2,2-dimethyl-propyl)-2-(4-methyl-pyrazol-1-yl)-benzylamino]-2-hydroxy-propyl}-acetamide, N-(4-(2-(1H-indazol-1-yl)-5-neopentylbenzylamino)-1-(3,5-difluorophenyl)-3-hydroxybutan-2-yl)acetamide, N-(1-(3,5-difluorophenyl)-3-hydroxy-4-(5-neopentyl-2-(1H-1,2,3-triazol-1-yl)benzylamino)butan-2-yl)acetamide, N-(1-(3,5-difluorophenyl)-3-hydroxy-4-(5-neopentyl-2-(2H-1,2,3-triazol-2-yl)benzylamino)butan-2-yl)acetamide, N-(1-(3,5-difluorophenyl)-3-hydroxy-4-(5-neopentyl-2-(1H-1,2,4-triazol-1-yl)benzylamino)butan-2-yl)acetamide, N-(1-(3,5-difluorophenyl)-3-hydroxy-4-(5-neopentyl-2-(pyrrolidin-1-yl)benzylamino)butan-2-yl)acetamide, N-(1-(3,5-difluorophenyl)-3-hydroxy-4-(2-(2-mercapto-1H-imidazol-1-yl)-5-neopentylbenzylamino)butan-2-yl)acetamide, methyl 3-(1-(2-((3-acetamido-4-(3,5-difluorophenyl)-2-hydroxybutylamino)methyl)-4-neopentylphenyl)-1H-imidazol-4-yl)acrylate, 3-(1-(2-((3-acetamido-4-(3,5-difluorophenyl)-2-hydroxybutylamino)methyl)-4-neopentylphenyl)-1H-imidazol-4-yl)-2-aminopropanoic acid, N-(1-(3,5-difluorophenyl)-3-hydroxy-4-(2-(3-hydroxypyrrolidin-1-yl)-5-neopentylbenzylamino)butan-2-yl)acetamide, and N-(1-(3,5-difluorophenyl)-3-hydroxy-4-(5-neopentyl-2-(piperidin-1-yl)benzylamino)butan-2-yl)acetamide, or a pharmaceutically acceptable salt thereof.

The present invention encompasses methods of treatment using compounds with structural characteristics designed for interacting with their target molecules. Such characteristics include at least one moiety capable of interacting with at least one subsite of beta-secretase. Such characteristics also include at least one moiety capable of enhancing the interaction between the target and at least one subsite of beta-secretase.

Accordingly, the compounds of formula (I) incorporate biaryl moieties at R_{C}. Compounds with such moieties possess structural characteristics that corresponds to desired properties such as increased bioavailability, efficacy, and/or selectivity.

It is preferred that the compounds of formula (I) are efficacious. For example, it is preferred that the compounds of formula (I) decrease the level of beta-secretase using low dosages of the compounds. Preferably, the compounds of formula (I) decrease the level of A-beta by at least 10% using dosages of about 100 mg/kg. It is more preferred that the compounds of formula (I) decrease the level of A-beta by at least 10% using dosages of less than 100 mg/kg. It is also more preferred that the compounds of formula (I) decrease the level of A-beta by greater than 10% using dosages of about 100 mg/kg. It is most preferred that the compounds of formula (I) decrease the level of A-beta by greater than 10% using dosages of less than 100 mg/kg.

Another embodiment of the present invention is to provide methods of preventing or treating conditions associated with amyloidosis using compounds with increased oral bioavailability (increased F values).

Accordingly, an embodiment of the present invention is also directed to methods for preventing or treating conditions associated with amyloidosis, comprising administering to a host a therapeutically effective amount of at least one compound of formula (I), or a pharmaceutically acceptable salt thereof, wherein R₁, R₂, and R_{C} are as previously defined, and wherein the compound has an F value of at least 10%.

Investigation of potential beta-secretase inhibitors produced compounds with increased selectivity for beta-secretase over other aspartyl proteases such as cathepsin D (catD), cathepsin E (catE), HIV protease, and renin. Selectivity was calculated as a ratio of inhibition (IC₅₀) values in which the inhibition of beta-secretase was compared to the inhibition of other aspartyl proteases. A compound is selective when the IC₅₀ value (i.e., concentration required for 50% inhibition) of a desired target (e.g., beta-secretase) is less than the IC₅₀ value of a secondary target (e.g., catD). Alternatively, a compound is selective when its binding affinity is greater for its desired target (e.g., beta-secretase) versus a secondary target (e.g., catD). Accordingly, methods of treatment include administering selective compounds of formula (I) having a lower IC₅₀ value for inhibiting beta-secretase, or greater binding affinity for beta-secretase, than for other aspartyl proteases such as catD, catE, HIV protease, or renin. A selective compound is also capable of producing a higher ratio of desired effects to adverse effects, resulting in a safer method of treatment.

In another embodiment, the host is a cell.

In another embodiment, the host is an animal.

In another embodiment, the host is human.

In another embodiment, at least one compound of formula (I) is administered in combination with a pharmaceutically acceptable carrier or diluent.

In another embodiment, the pharmaceutical compositions comprising compounds of formula (I) can be used to treat a wide variety of disorders or conditions including Alzheimer's disease, Down's syndrome or Trisomy 21 (including mild cognitive impairment (MCI) Down's syndrome), hereditary cerebral hemorrhage with amyloidosis of the Dutch type, chronic inflammation due to amyloidosis, prion diseases (including Creutzfeldt-Jakob disease, Gerstmann-Straussler syndrome, kuru scrapie, and animal scrapie), Familial Amyloidotic Polyneuropathy, cerebral amyloid angiopathy, other degenerative dementias including dementias of mixed vascular and degenerative origin, dementia associated with Parkinson's disease, dementia associated with progressive supranuclear palsy and dementia associated with cortical basal degeneration, diffuse Lewy body type of Alzheimer's disease, and frontotemporal dementias with parkinsonism (FTDP).

In another embodiment, the condition is Alzheimer's disease.

In another embodiment, the condition is dementia.

When treating or preventing these diseases, the methods of the present invention can either employ the compounds of formula (I) individually or in combination, as is best for the patient.

In treating a patient displaying any of the conditions discussed above, a physician may employ a compound of formula (I) immediately and continue administration indefinitely, as needed. In treating patients who are not diagnosed as having Alzheimer's disease, but who are believed to be at substantial risk for it, the physician may start treatment when the patient first experiences early pre-Alzheimer's symptoms, such as memory or cognitive problems associated with aging. In addition, there are some patients who may be determined to be at risk for developing Alzheimer's disease through the detection of a genetic marker such as APOE4 or other biological indicators that are predictive for Alzheimer's disease and related conditions. In these situations, even though the patient does not have symptoms of the disease or condition, administration of the compounds of formula (I) may be started before symptoms appear, and treatment may be continued indefinitely to prevent or delay the onset of the disease. Similar protocols are provided for other diseases and conditions associated with amyloidosis, such as those characterized by dementia.

In an embodiment, the methods of preventing or treating conditions associated with amyloidosis, comprising administering to a host in need thereof a composition comprising a therapeutically effective amount of at least one compound of formula (I), may include beta-secretase complexed with at least one compound of formula (I), or a pharmaceutically acceptable salt thereof.

Another embodiment of the present invention is a method of preventing or treating the onset of Alzheimer's disease comprising administering to a patient a therapeutically effective amount of at least one compound of formula (I), or a pharmaceutically acceptable salt thereof, wherein R₁, R₂, and R_{C} are as previously defined.

Another embodiment of the present invention is a method of preventing or treating the onset of dementia comprising administering to a patient a therapeutically effective amount of at least one compound of formula (I), or a pharmaceutically acceptable salt thereof, wherein R₁, R₂, and R_{C} are as previously defined.

Another embodiment of the present invention is a method of preventing or treating conditions associated with amyloidosis by administering to a host in need thereof an effective amount of at least one compound of formula (I), or a pharmaceutically acceptable salt thereof, wherein R₁, R₂, and R_{C} are as previously defined.

Another embodiment of the present invention is a method of preventing or treating Alzheimer's disease by administering to a host in need thereof an effective amount of at least one compound of formula (I), or a pharmaceutically acceptable salt thereof, wherein R₁, R₂, and R_{C} are as previously defined.

Another embodiment of the present invention is a method of preventing or treating dementia by administering to a host in need thereof an effective amount of at least one compound of formula (I), or a pharmaceutically acceptable salt thereof, wherein R₁, R₂, and R_{C} are as previously defined.

Another embodiment of the present invention is a method of inhibiting beta-secretase activity in a cell. This method comprises administering to the cell an effective amount of at least one compound of formula (I), or a pharmaceutically acceptable salt thereof, wherein R₁, R₂, and R_{C} are as previously defined.

Another embodiment of the present invention is a method of inhibiting beta-secretase activity in a host. This method comprises administering to the host an effective amount of at least one compound of formula (I), or a pharmaceutically acceptable salt thereof, wherein R₁, R₂, and R_{C} are as previously defined.

Another embodiment of the present invention is a method of inhibiting beta-secretase activity in a host. This method comprises administering to the host an effective amount of at least one compound of formula (I), or a pharmaceutically acceptable salt thereof, wherein R₁, R₂, and R_{C} are as previously defined, and wherein the host is a human.

Another embodiment of the present invention is methods of affecting beta-secretase-mediated cleavage of amyloid precursor protein in a patient, comprising administering a therapeutically effective amount of at least one compound of formula (I), or a pharmaceutically acceptable salt thereof, wherein R₁, R₂, and R_{C} are as previously defined.

Another embodiment of the present invention is a method of inhibiting cleavage of amyloid precursor protein at a site between Met596 and Asp597 (numbered for the APP-695 amino acid isotype), or at a corresponding site of an isotype or mutant thereof, comprising administering a therapeutically effective amount of at least one compound of formula (I), or a pharmaceutically acceptable salt thereof, wherein R₁, R₂, and R_{C} are as previously defined.

Another embodiment of the present invention is a method of inhibiting cleavage of amyloid precursor protein or mutant thereof at a site between amino acids, comprising administering a therapeutically effective amount of at least one compound of formula (I), or a pharmaceutically acceptable salt thereof, wherein R₁, R₂, and R_{C} are as previously defined, and wherein the site between amino acids corresponds to
between Met652 and Asp653 (numbered for the APP-751 isotype),
between Met671 and Asp672 (numbered for the APP-770 isotype),
between Leu596 and Asp597 of the APP-695 Swedish Mutation,
between Leu652 and Asp653 of the APP-751 Swedish Mutation, or
between Leu671 and Asp672 of the APP-770 Swedish Mutation.

Another embodiment of the present invention is a method of inhibiting production of A-beta, comprising administering to a patient a therapeutically effective amount of at least one compound of formula (I), or a pharmaceutically acceptable salt thereof, wherein R₁, R₂, and R_{C} are as previously defined.

Another embodiment of the present invention is a method of preventing or treating deposition of A-beta, comprising administering a therapeutically effective amount of at least one compound of formula (I), or a pharmaceutically acceptable salt thereof, wherein R₁, R₂, and R_{C} are as previously defined.

Another embodiment of the present invention is a method of preventing, delaying, halting, or reversing a disease characterized by A-beta deposits or plaques, comprising administering a therapeutically effective amount of at least one compound of formula (I), or a pharmaceutically acceptable salt thereof, wherein R₁, R₂, and R_{C} are as previously defined.

In an embodiment, the A-beta deposits or plaques are in a human brain.

Another embodiment of the present invention is a method of preventing, delaying, halting, or reversing a condition associated with a pathological form of A-beta in a host comprising administering to a patient in need thereof an effective amount of at least one compound of formula (I), or a pharmaceutically acceptable salt thereof, wherein R₁, R₂, and R_{C} are as previously defined.

Another embodiment of the present invention is a method of inhibiting the activity of at least one aspartyl protease in a patient in need thereof, comprising administering a therapeutically effective amount of at least one compound of formula (I), or a pharmaceutically acceptable salt thereof to the patient, wherein R₁, R₂, and R_{C} are as previously defined.

In an embodiment, the at least one aspartyl protease is beta-secretase.

Another embodiment of the present invention is a method of interacting an inhibitor with beta-secretase, comprising administering to a patient in need thereof a therapeutically effective amount of at least one compound of formula (I), or a pharmaceutically acceptable salt thereof, wherein R₁, R₂, and R_{C} are as previously defined, and wherein the at least one compound interacts with at least one beta-secretase subsite such as S1, S1′, or S2′.

Another embodiment of the present invention is a method of selecting compounds of formula (I) wherein the pharmacokinetic parameters are adjusted for an increase in desired effect (e.g., increased brain uptake).

Another embodiment of the present invention is a method of selecting compounds of formula (I) wherein Cₘₐₓ, Tₘₐₓ, and/or half-life are adjusted to provide for maximum efficacy.

Another embodiment of the present invention is a method of treating a condition in a patient, comprising administering a therapeutically effective amount of at least one compound of formula (I), or a pharmaceutically acceptable salt, derivative or biologically active metabolite thereof, to the patient, wherein R₁, R₂, and R_{C} are as previously defined.

In an embodiment, the condition is Alzheimer's disease.

In another embodiment, the condition is dementia.

In another embodiment of the present invention, the compounds of formula (I) are administered in oral dosage form. The oral dosage forms are generally administered to the patient 1, 2, 3, or 4 times daily. It is preferred that the compounds be administered either three or fewer times daily, more preferably once or twice daily. It is preferred that, whatever oral dosage form is used, it be designed so as to protect the compounds from the acidic environment of the stomach. Enteric coated tablets are well known to those skilled in the art. In addition, capsules filled with small spheres, each coated to be protected from the acidic stomach, are also well known to those skilled in the art.

Therapeutically effective amounts include, for example, oral administration from about 0.1 mg/day to about 1,000 mg/day, parenteral, sublingual, intranasal, intrathecal administration from about 0.2 to about 100 mg/day, depot administration and implants from about 0.5 mg/day to about 50 mg/day, topical administration from about 0.5 mg/day to about 200 mg/day, and rectal administration from about 0.5 mg/day to about 500 mg/day.

When administered orally, an administered amount therapeutically effective to inhibit beta-secretase activity, to inhibit A-beta production, to inhibit A-beta deposition, or to treat or prevent Alzheimer's disease is from about 0.1 mg/day to about 1,000 mg/day.

In various embodiments, the therapeutically effective amount may be administered in, for example, pill, tablet, capsule, powder, gel, or elixir form, and/or combinations thereof. It is understood that, while a patient may be started at one dose or method of administration, that dose or method of administration may be varied over time as the patient's condition changes.

Another embodiment of the present invention is a method of prescribing a medication for preventing, delaying, halting, or reversing disorders, conditions or diseases associated with amyloidosis. The method includes identifying in a patient symptoms associated with disorders, conditions or diseases associated with amyloidosis, and prescribing at least one dosage form of at least one compound of formula (I), or a pharmaceutically acceptable salt, to the patient, wherein R₁, R₂, and R_{C} are as previously defined.

Another embodiment of the present invention, is an article of manufacture, comprising (a) at least one dosage form of at least one compound of formula (I), or a pharmaceutically acceptable salt thereof, wherein R₁, R₂, and R_{C} are as previously defined, (b) a package insert providing that a dosage form comprising a compound of formula (I) should be administered to a patient in need of therapy for at least one disorder, condition or disease associated with amyloidosis, and (c) at least one container in which at least one dosage form of at least one compound of formula (I) is stored.

Another embodiment of the present invention is a packaged pharmaceutical composition for treating conditions related to amyloidosis, comprising (a) a container which holds an effective amount of at least one compound of formula (I), or a pharmaceutically acceptable salt thereof, and (b) instructions for using the pharmaceutical composition.

Another embodiment of the present invention is an article of manufacture, comprising (a) a therapeutically effective amount of at least one compound of formula (I), or a stereoisomer, or pharmaceutically acceptable salt thereof, wherein R₁, R₂, and R_{C} are as previously defined, (b) a package insert providing an oral dosage form should be administered to a patient in need of therapy for at least one disorder, condition or disease associated with amyloidosis, and (c) at least one container comprising at least one oral dosage form of at least one compound of formula (I).

Another embodiment of the present invention is an article of manufacture, comprising (a) at least one oral dosage form of at least one compound of formula (I), or a pharmaceutically acceptable salt thereof, wherein R₁, R₂, and R_{C} are as previously defined, in a dosage amount ranging from about 2 mg to about 1000 mg, associated with (b) a package insert providing that an oral dosage form comprising a compound of formula (I) in a dosage amount ranging from about 2 mg to about 1000 mg should be administered to a patient in need of therapy for at least one disorder, condition or disease associated with amyloidosis, and (c) at least one container in which at least one oral dosage form of at least one compound of formula (I) in a dosage amount ranging from about 2 mg to about 1000 mg is stored.

Another embodiment of the present invention is an article of manufacture, comprising (a) at least one oral dosage form of at least one compound of formula (I) in a dosage amount ranging from about 2 mg to about 1000 mg in combination with (b) at least one therapeutically active agent, associated with (c) a package insert providing that an oral dosage form comprising a compound of formula (I) in a dosage amount ranging from about 2 mg to about 1000 mg in combination with at least one therapeutically active agent should be administered to a patient in need of therapy for at least one disorder, condition or disease associated with amyloidosis, and (d) at least one container in which at least one dosage form of at least one compound of formula (I) in a dosage amount ranging from about 2 mg to about 1000 mg in combination with a therapeutically active agent is stored.

Another embodiment of the present invention is an article of manufacture, comprising (a) at least one parenteral dosage form of at least one compound of formula (I) in a dosage amount ranging from about 0.2 mg/mL to about 50 mg/mL, associated with (b) a package insert providing that a parenteral dosage form comprising a compound of formula (I) in a dosage amount ranging from about 0.2 mg/mL to about 50 mg/mL should be administered to a patient in need of therapy for at least one disorder, condition or disease associated with amyloidosis, and (c) at least one container in which at least one parenteral dosage form of at least one compound of formula (I) in a dosage amount ranging from about 0.2 mg/mL to about 50 mg/mL is stored.

Another embodiment of the present invention is an article of manufacture comprising (a) a medicament comprising an effective amount of at least one compound of formula (I) in combination with active and/or inactive pharmaceutical agents, (b) a package insert providing that an effective amount of at least one compound of formula (I) should be administered to a patient in need of therapy for at least one disorder, condition or disease associated with amyloidosis, and (c) a container in which a medicament comprising an effective amount of at least one compound of formula (I) in combination with therapeutically active and/or inactive agents is stored.

In an embodiment, the therapeutically active agent is selected from an antioxidant, an anti-inflammatory, a gamma-secretase inhibitor, a neurotropic agent, an acetyl cholinesterase inhibitor, a statin, an A-beta or fragment thereof, and/or an anti-A-beta antibody.

Another embodiment is a kit comprising at least one component independently selected from: (a) at least one dosage form of a formula (I) compound; (b) at least one container in which at least one dosage form of a formula (I) compound is stored; (c) a package insert (optionally containing information of the dosage amount and duration of exposure of a dosage form containing at least one compound of formula (I) and optionally providing that the dosage form should be administered to a patient in need of therapy for at least one disorder, condition or disease associated with amyloidosis; and (d) at least one therapeutically active agent (optionally selected from an antioxidant, an anti-inflammatory, a gamma-secretase inhibitor, a neurotrophic agent, an acetyl cholinesterase inhibitor, a statin, an A-beta or fragment thereof, and an anti-A-beta antibody).

Another embodiment of the present invention is a method of producing A-beta-secretase complex comprising exposing beta-secretase to a compound of formula (I), wherein R₁, R₂, and R_{C} are as previously defined, or a pharmaceutically acceptable salt thereof, in a reaction mixture under conditions suitable for the production of the complex.

Another embodiment of the present invention is a manufacture of a medicament for preventing, delaying, halting, or reversing Alzheimer's disease, comprising adding an effective amount of at least one compound of formula (I) to a pharmaceutically acceptable carrier.

Another embodiment of the present invention is a method of selecting a beta-secretase inhibitor comprising targeting the moieties of at least one formula (I) compound, or a pharmaceutically acceptable salt thereof, to interact with at least one beta-secretase subsite such as but not limited to S1, S1′, or S2′.

The methods of treatment described herein include administering the compounds of formula (I) orally, parenterally (via intravenous injection (IV), intramuscular injection (IM), depo-IM, subcutaneous injection (SC or SQ), or depo-SQ), sublingually, intranasally (inhalation), intrathecally, topically, or rectally. Dosage forms known to those skilled in the art are suitable for delivery of the compounds of formula (I).

In treating or preventing the above diseases, the compounds of formula (I) are administered using a therapeutically effective amount. The therapeutically effective amount will vary depending on the particular compound used and the route of administration, as is known to those skilled in the art.

The compositions are preferably formulated as suitable pharmaceutical preparations, such as for example, pill, tablet, capsule, powder, gel, or elixir form, and/or combinations thereof, for oral administration or in sterile solutions or suspensions for parenteral administration. Typically the compounds described above are formulated into pharmaceutical compositions using techniques and/or procedures well known in the art.

For example, a therapeutically effective amount of a compound or mixture of compounds of formula (I), or a physiologically acceptable salt, is combined with a physiologically acceptable vehicle, carrier, binder, preservative, stabilizer, flavor, and the like, in a unit dosage form as called for by accepted pharmaceutical practice and as defined herein. The amount of active substance in those compositions or preparations is such that a suitable dosage in the range indicated is obtained. The compound concentration is effective for delivery of an amount upon administration that lessens or ameliorates at least one symptom of the disorder for which the compound is administered. For example, the compositions can be formulated in a unit dosage form, each dosage containing from about 2 mg to about 1000 mg.

The active ingredient may be administered in a single dose, or may be divided into a number of smaller doses to be administered at intervals of time. It is understood that the precise dosage and duration of treatment is a function of the disease or condition being treated and may be determined empirically using known testing protocols or by extrapolation from in vivo or in vitro test data. Also, concentrations and dosage values may vary with the severity of the condition to be alleviated. It is also to be understood that the precise dosage and treatment regimens may be adjusted over time according to the individual need and the professional judgment of the person administering or supervising the administration of the compositions, and that the concentration ranges set forth herein are exemplary only and are not intended to limit the scope or practice of the claimed compositions. A dosage and/or treatment method for any particular patient also may depend on, for example, the age, weight, sex, diet, and/or health of the patient, the time of administration, and/or any relevant drug combinations or interactions.

To prepare compositions to be employed in the methods of treatment, at least one compound of formula (I) is mixed with a suitable pharmaceutically acceptable carrier. Upon mixing or addition of the compound(s), the resulting mixture may be a solution, suspension, emulsion, or the like. Liposomal suspensions may also be-suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. An effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. Additionally, the active materials can also be mixed with other active materials that do not impair the desired action, or with materials that supplement the desired action, or have another action. For example, the compounds of formula (I) may be formulated as the sole pharmaceutically active ingredient in the composition or may be combined with other active ingredients.

Where the compounds exhibit insufficient solubility, methods for solubilizing may be used. Such methods are known and include, for example, using co-solvents (such as dimethylsulfoxide, (DMSO)), using surfactants (such as Tween®), and/or dissolution in aqueous sodium bicarbonate. Derivatives of the compounds, such as salts, metabolites, and/or pro-drugs, may also be used in formulating effective pharmaceutical compositions. Such derivatives may improve the pharmacokinetic properties of treatment administered.

The compounds of formula (I) may be prepared with carriers that protect them against rapid elimination from the body, such as time-release formulations or coatings. Such carriers include controlled release formulations, such as, for example, microencapsulated delivery systems and the like. The active compound is included in the pharmaceutically acceptable carrier in an amount sufficient to exert a therapeutically useful effect in the absence of undesirable side effects on the patient treated. Alternatively, the active compound is included in an amount sufficient to exert a therapeutically useful effect and/or minimize the severity and form of undesirable side effects. The therapeutically effective concentration may be determined empirically by testing the compounds in known in vitro and/or in vivo model systems for the treated disorder.

The tablets, pills, capsules, troches, and the like may contain a binder (e.g., gum tragacanth, acacia, corn starch, gelatin, and the like); a vehicle (e.g., microcrystalline cellulose, starch, lactose, and the like); a disintegrating agent (e.g., alginic acid, corn starch, and the like); a lubricant (e.g., magnesium stearate and the like); a gildant (e.g., colloidal silicon dioxide and the like); a sweetening agent (e.g., sucrose, saccharin, and the like); a flavoring agent (e.g., peppermint, methyl salicylate, fruit flavoring, and the like); compounds of a similar nature, and/or mixtures thereof.

When the dosage unit form is a capsule, it can contain, in addition to material described above, a liquid carrier such as a fatty oil. Additionally, dosage unit forms can contain various other materials, which modify the physical form of the dosage unit, for example, coatings of sugar or other enteric agents. A method of treatment can also administer the compound as a component of an elixir, suspension, syrup, wafer, chewing gum or the like. A syrup may contain, in addition to the active compounds, sucrose as a sweetening agent, flavors, preservatives, dyes and/or colorings.

The methods of treatment may employ at least one carrier that protects the compound against rapid elimination from the body, such as time-release formulations or coatings. Such carriers include controlled release formulations, such as, for example, implants or microencapsulated delivery systems, or biodegradable, biocompatible polymers such as collagen, ethylene vinyl acetate, polyanhydrides, polyglycolic acid, polyorthoesters, polylactic acid, and the like. Methods for preparation of such formulations are known to those in the art.

When orally administered, the compounds of the present invention can be administered in usual dosage forms for oral administration as is well known to those skilled in the art. These dosage forms include the usual solid unit dosage forms of tablets and capsules as well as liquid dosage forms such as solutions, suspensions, and elixirs. When solid dosage forms are used, it is preferred that they be of the sustained release type so that the compounds of the present invention need to be administered only once or twice daily. When liquid oral dosage forms are used, it is preferred that they be of about 10 mL to about 30 mL each. Multiple doses may be administered daily.

The methods of treatment may also employ a mixture of the active materials and other active or inactive materials that do not impair the desired action, or with materials that supplement the desired action.

Solutions or suspensions used for parenteral, intradermal, subcutaneous, or topical application can include a sterile diluent (e.g., water for injection, saline solution, fixed oil, and the like); a naturally occurring vegetable oil (e.g., sesame oil, coconut oil, peanut oil, cottonseed oil, and the like); a synthetic fatty vehicle (e.g., ethyl oleate, polyethylene glycol, glycerine, propylene glycol, and the like, including other synthetic solvents); antimicrobial agents (e.g., benzyl alcohol, methyl parabens, and the like); antioxidants (e.g., ascorbic acid, sodium bisulfite, and the like); chelating agents (e.g., ethylenediaminetetraacetic acid (EDTA), and the like); buffers (e.g., acetates, citrates, phosphates, and the like); and/or agents for the adjustment of tonicity (e.g., NaCl, dextrose, and the like); or mixtures thereof.

Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and the like, and mixtures thereof. Liposomal suspensions including tissue-targeted liposomes may also be suitable as pharmaceutically acceptable carriers. These may be prepared according to methods known, for example, as described in U.S. Pat. No. 4,522,811.

The methods of treatment include delivery of the compounds of the present invention in a nano crystal dispersion formulation. Preparation of such formulations is described, for example, in U.S. Pat. No. 5,145,684. Nano crystalline dispersions of Human Immunodeficiency Viral (HIV) protease inhibitors and their method of use are described in U.S. Pat. No. 6,045,829. The nano crystalline formulations typically afford greater bioavailability of drug compounds.

The methods of treatment include administration of the compounds parenterally, for example, by IV, IM, SC, or depo-SC. When administered parenterally, a therapeutically effective amount of about 0.2 mg/mL to about 50 mg/mL is preferred. When a depot or IM formulation is used for injection once a month or once every two weeks, the preferred dose should be about 0.2 mg/mL to about 50 mg/mL.

The methods of treatment include administration of the compounds sublingually. When given sublingually, the compounds of the present invention should be given one to four times daily in the amounts described above for IM administration.

The methods of treatment include administration of the compounds intranasally. When given by this route, the appropriate dosage forms are a nasal spray or dry powder, as is known to those skilled in the art. The dosage of the compounds of the present invention for intranasal administration is the amount described above for IM administration.

The methods of treatment include administration of the compounds intrathecally. When given by this route the appropriate dosage form can be a parenteral dosage form as is known to those skilled in the art. The dosage of the compounds of the present invention for intrathecal administration is the amount described above for IM administration.

The methods of treatment include administration of the compounds topically. When given by this route, the appropriate dosage form is a cream, ointment, or patch. When topically administered, the dosage is from about 0.2 mg/day to about 200 mg/day. Because the amount that can be delivered by a patch is limited, two or more patches may be used. The number and size of the patch is not important. What is important is that a therapeutically effective amount of a compound of the present invention be delivered as is known to those skilled in the art. The compound can be administered rectally by suppository as is known to those skilled in the art. When administered by suppository, the therapeutically effective amount is from about 0.2 mg to about 500 mg.

The methods of treatment include administration of the compounds by implants as is known to those skilled in the art. When administering a compound of the present invention by implant, the therapeutically effective amount is the amount described above for depot administration.

Given a particular compound of the present invention and/or a desired dosage form and medium, one skilled in the art would know how to prepare and administer the appropriate dosage form and/or amount.

The methods of treatment include use of the compounds of the present invention, or acceptable pharmaceutical salts thereof, in combination, with each other or with other therapeutic agents, to treat or prevent the conditions listed above. Such agents or approaches include acetylcholinesterase inhibitors such as tacrine (tetrahydroaminoacridine, marketed as COGNEX®), donepezil hydrochloride, (marketed as Aricept®) and rivastigmine (marketed as Exelon®); gamma-secretase inhibitors; anti-inflammatory agents such as cyclooxygenase 11 inhibitors; anti-oxidants such as Vitamin E or ginkolides; immunological approaches, such as, for example, immunization with A-beta peptide or administration of anti-A-beta peptide antibodies; statins; and direct or indirect neurotropic agents such as Cerebrolysin®, AIT-082 (Emilien, 2000, Arch. Neurol. 57:454), and other neurotropic agents; and complexes with beta-secretase or fragments thereof.

Additionally, the methods of treatment also employ the compounds of the present invention with inhibitors of P-glycoprotein (P-gp). P-gp inhibitors and the use of such compounds are known to those skilled in the art. See, for example, Cancer Research, 53, 4595-4602 (1993), Clin. Cancer Res., 2, 7-12 (1996), Cancer Research, 56, 4171-4179 (1996), International Publications WO 99/64001 and WO 01/10387. The blood level of the P-gp inhibitor should be such that it exerts its effect in inhibiting P-gp from decreasing brain blood levels of the compounds of formula (I). To that end the P-gp inhibitor and the compounds of formula (I) can be administered at the same time, by the same or different route of administration, or at different times. Given a particular compound of formula (I), one skilled in the art would know whether a P-gp inhibitor is desirable for use in the method of treatment, which P-gp inhibitor should be used, and how to prepare and administer the appropriate dosage form and/or amount.

Suitable P-gp inhibitors include cyclosporin A, verapamil, tamoxifen, quinidine, Vitamin E-TGPS, ritonavir, megestrol acetate, progesterone, rapamycin, 10,11-methanodibenzosuberane, phenothiazines, acridine derivatives such as GF120918, FK506, VX-710, LY335979, PSC-833, GF-102,918, quinoline-3-carboxylic acid (2-{4-[2-(6,7-dimethyl-3,4-dihydro-1H-isoquinoline-2-yl)-ethyl]phenylcarbamoyl}-4,5-dimethylphenyl)-amide (Xenova), or other compounds. Compounds that have the same function and therefore achieve the same outcome are also considered to be useful.

The P-gp inhibitors can be administered orally, parenterally, (via IV, IM, depo-IM, SQ, depo-SQ), topically, sublingually, rectally, intranasally, intrathecally, or by implant.

The therapeutically effective amount of the P-gp inhibitors is from about 0.1 mg/kg to about 300 mg/kg daily, preferably about 0.1 mg/kg to about 150 mg/kg daily. It is understood that while a patient may be started on one dose, that dose may vary over time as the patient's condition changes.

When administered orally, the P-gp inhibitors can be administered in usual dosage forms for oral administration as is known to those skilled in the art. These dosage forms include the usual solid unit dosage forms of tablets or capsules as well as liquid dosage forms such as solutions, suspensions or elixirs. When the solid dosage forms are used, it is preferred that they be of the sustained release type so that the P-gp inhibitors need to be administered only once or twice daily. The oral dosage forms are administered to the patient one through four times daily. It is preferred that the P-gp inhibitors be administered either three or fewer times a day, more preferably once or twice daily. Hence, it is preferred that the P-gp inhibitors be administered in solid dosage form and further it is preferred that the solid dosage form be a sustained release form which permits once or twice daily dosing. It is preferred that the dosage form used is designed to protect the P-gp inhibitors from the acidic environment of the stomach. Enteric coated tablets are well known to those skilled in the art. In addition, capsules filled with small spheres each coated to protect from the acidic stomach, are also well known to those skilled in the art.

In addition, the P-gp inhibitors can be administered parenterally. When administered parenterally they can be administered via IV, IM, depo-IM, SQ or depo-SQ.

The P-gp inhibitors can be given sublingually. When given sublingually, the P-gp inhibitors should be given one through four times daily in the same amount as for IM administration.

The P-gp inhibitors can be given intranasally. When given by this route of administration, the appropriate dosage forms are a nasal spray or dry powder as is known to those skilled in the art. The dosage of the P-gp inhibitors for intranasal administration is the same as for IM administration.

The P-gp inhibitors can be given intrathecally. When given by this route of administration the appropriate dosage form can be a parenteral dosage form as is known to those skilled in the art.

The P-gp inhibitors can be given topically. When given by this route of administration, the appropriate dosage form is a cream, ointment or patch. Because of the amount of the P-gp inhibitors needed to be administered the patch is preferred. However, the amount that can be delivered by a patch is limited. Therefore, two or more patches may be required. The number and size of the patch is not important; what is important is that a therapeutically effective amount of the P-gp inhibitors be delivered as is known to those skilled in the art.

The P-gp inhibitors can be administered rectally by suppository or by implants, both of which are known to those skilled in the art.

It should be apparent to one skilled in the art that the exact dosage and frequency of administration will depend on the particular compounds of the present invention administered, the particular condition being treated, the severity of the condition being treated, the age, weight, or general physical condition of the particular patient, or any other medication the individual may be taking as is well known to administering physicians who are skilled in this art.

### EXPERIMENTAL PROCEDURES

The compounds and the methods of treatment of the present invention can generally be prepared by one skilled in the art based on knowledge of the compound's chemical structure. The chemistry for the preparation of compounds employed in the methods of treatment of this invention is known to those skilled in the art. In fact, there is more than one process to prepare the compounds employed in the methods of treatment of the present invention. Specific examples of methods of preparing the compounds of the present invention can be found in the art. For examples, see Zuccarello et al., J. Org. Chem. 1998, 63, 4898-4906; Benedetti et al., J. Org. Chem. 1997, 62, 9348-9353; Kang et al., J. Org. Chem. 1996, 61, 5528-5531; Kempf et al., J. Med. Chem. 1993, 36, 320-330; Lee et al., J. Am. Chem. Soc. 1999, 121, 1145-1155; and references cited therein; Chem. Pharm. Bull. (2000), 48(11), 1702-1710; J. Am. Chem. Soc. (1974), 96(8), 2463-72; Ind. J. Chem., §B: Organic Chemistry Including Medicinal Chemistry (2003), 42B(4), 910-915; and J. Chem. Soc. §C: Organic (1971), (9), 1658-10. See also U.S. Pat. Nos. 6,150,530, 5,892,052, 5,696,270, and 5,362,912, and references cited therein, which are incorporated herein by reference.

¹H and ¹³C NMR spectra were obtained on a Varian 400 MHz, Varian 300 MHz, or Bruker 300 MHz instrument. HPLC samples were analyzed using a YMC ODS-AQ S-3 120 A 3.0×50 mm cartridge, with a standard gradient from 5% acetonitrile containing 0.01% heptafluorobutyric acid (HFBA) and 1% isopropanol in water containing 0.01% HFBA to 95% acetonitrile containing 0.01% HFBA and 1% isopropanol in water containing 0.01% HFBA over 5 min. Mass spec samples were performed with electron spray ionization (ESI).

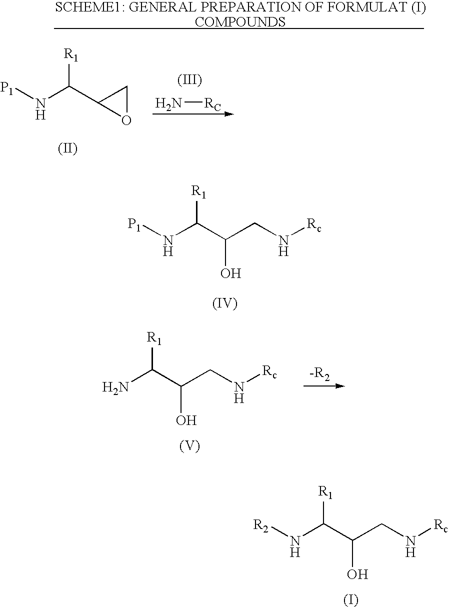

The general synthesis of compounds of formula (I) is shown in Scheme 1. First, epoxides (II), which were derived from amino acids and are known in the art (see Luly, J. R. et al. J. Org. Chem. 1987, 52, 1,487; Tucker, T. J. et al. J. Med. Chem. 1992, 35, 2525; Reeder, M. WO 02085877), were treated with 1.5-5 equivalents of primary amine H₂N—R_{C} (III) in an alcoholic solvent, such as ethanol, isopropanol, or sec-butanol to effect ring opening of the epoxide. In an embodiment, this reaction is performed at elevated temperatures from 40° C. to reflux. In another embodiment, the reaction is performed at reflux in isopropanol.

Second, the resulting amino alcohol (IV) was deprotected. Amino protecting groups were used when preparing compounds discussed herein. The chemistry of amino protection includes methods well known to those skilled in the art, e.g. those found in Greene, T. W.; Wuts, P. G. M. Protective Groups in Organic Synthesis, 3^{rd} ed., New York: John Wiley & Sons, Inc., 1999, and Kocienski, P. J. Protecting Groups. Stuttgart, FRG: G. T. Verlag, 1994.

The third step, addition of the group —C(O)—R₂ can be achieved by a variety of methods known in the art. For example, addition of the appropriate carboxylic acid with the use of BOP reagent (benzotriazolyl-N-hydroxytris(dimethylamino) phosphonium hexafluorophosphate) (Castro, B. et al. Tetrahedron Lett. 1975, 1219) or EDC (1-(3-dimethylaminopropyl)-3-ethylcarbodiimide hydrochloride) (Kimura, T. et al. Biopolymers 1981, 20,1823) to the amine yields an amido compound (I).

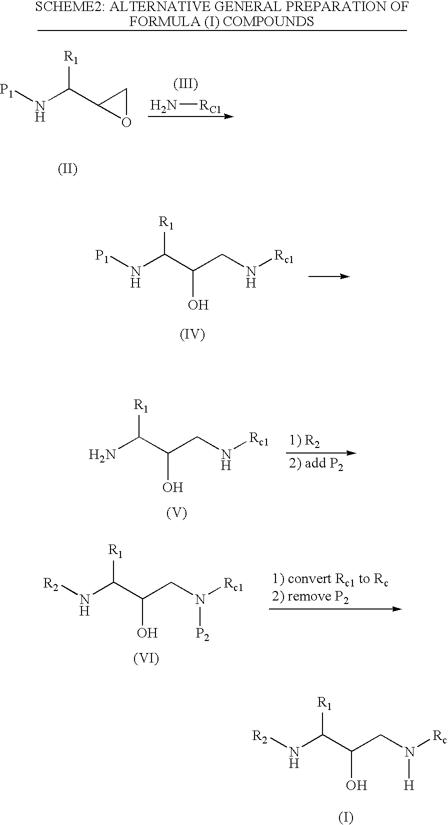

Alternatively, a common advanced intermediate (VI) may be used. In this scheme, a reactive group (R_{C1}) on compound (VI) may be converted to R_{C} to yield compounds (I). Epoxides (II) were treated with 1.5-5 equivalents of primary amine H₂N—R_{C1} (III) in an alcoholic solvent, such as ethanol, isopropanol, or sec-butanol to effect ring opening of the epoxide. In an embodiment, this reaction is prepared at elevated temperatures from 40° C. to reflux. In another embodiment, this reaction is performed at reflux in isopropanol. The resulting amino alcohol (IV) was then deprotected.

The coupling reaction proceeds as described in Scheme I. A protecting group may be used to protect the C-terminus amine group for the following step.

When R_{C1} contains a labile functional group, such as an aryl iodide, aryl bromide, aryl trifluoromethanesulfonate, or aryl boronic ester, which may be converted into R_{C} via transition metal-mediated coupling, this allows for the rapid synthesis of a variety of analogs (I). Such conversions may include Suzuki (aryl boronic acid or boronic ester and aryl halide), Negishi (arylzinc and aryl or vinyl halide), and Sonogashira (arylzinc and alkynyl halide) couplings. Subsequent to the coupling reaction, the protecting group P₂ is removed in methods known in the art to yield compounds (I).

### Example 1

### GENERAL PREPARATION OF BIARYL BETA-SECRETASE INHIBITOR USING SCHEME 2

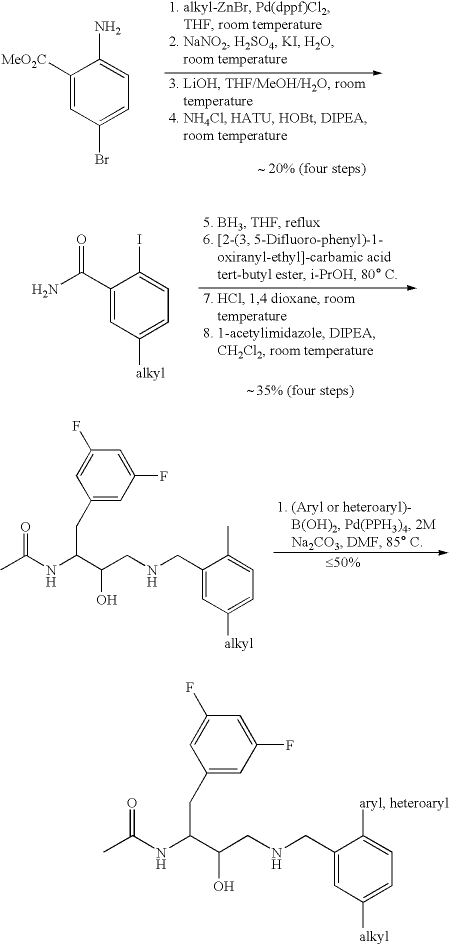

Suitable acids for use in deprotection (see steps 7 and 8) include trifluoroacetic acid in CH₂Cl₂ and 4 N HCl in ether or dioxane. Suitable acetylating reagents include, for example, acetylimidazole, diacetylmethoxylamine (Kikugawa, Y. et al. Tetrahedron Lett., 1990, 31, 243-246), and acetic acid/1-hydroxybenzotriazole/1-(3-dimethylaminopropyl)-3-ethylcarbodiimide.

### Example 2

### GENERAL PREPARATION OF BIARYL BETA-SECRETASE INHIBITORS USING SCHEME 1

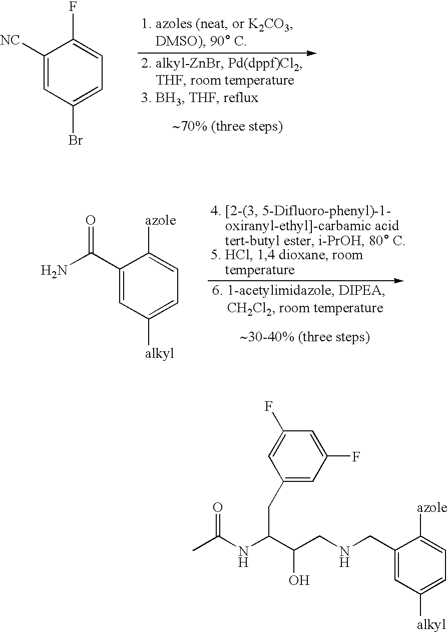

The Negishi coupling (see step 2) may be performed with 1.5-5 equivalents of alkylzinc halide (e.g., chloride, bromide, or iodide) reagent in ethereal solvent such as diethyl ether or tetrahydrofuran, at room temperature to 70° C. The coupling is facilitated by 2-20 mol % palladium catalyst. Appropriate catalysts include, for example, dichlorobis(triphenylphosphine)palladium(II), dichlorobis(tri-o-tolylphosphine) palladium(II), and [bis(diphenylphosphino)ferrocene]palladium(II) (1:1 complex with CH₂Cl₂).

Suitable reduction reagents for step 3 include, for example borane-dimethylsulfide complex, borane-tetrahydrofuran complex, borane-dimethylamine complex, lithium aluminum hydride, and hydrogen gas over palladium on carbon.

Suitable acids for use in deprotection (see steps 5 and 6) include, for example, trifluoroacetic acid in CH₂Cl₂ and 4 N HCl in ether or dioxane. Suitable acetylating reagents include, for example, acetylimidazole, diacetylmethoxylamine (Kikugawa, Y. et al. Tetrahedron Lett., 1990, 31, 243-246), and acetic acid /1-hydroxybenzotriazole /1-(3-dimethylamino propyl)-3-ethylcarbodiimide.

### Example 3

### PREPARATION OF BIARYL BETA-SECRETASE INHIBITORS USING SCHEME 1

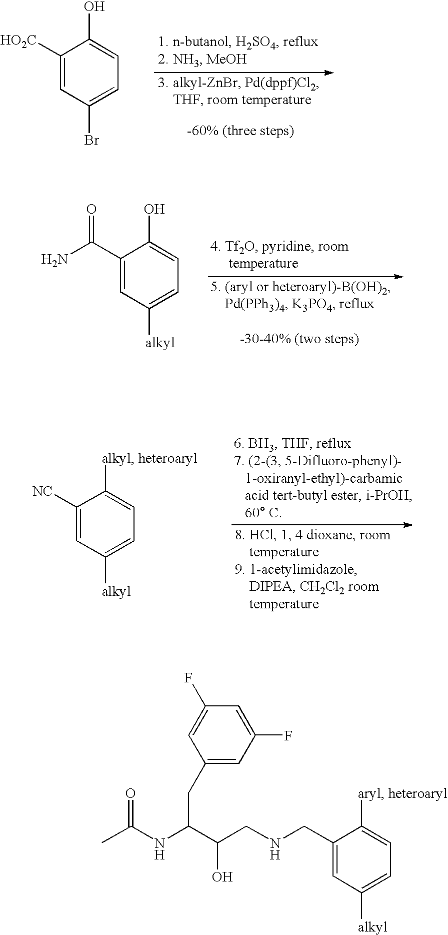

The Negishi coupling (see step 3) may be performed with 1.5-5 equivalents of alkylzinc halide (e.g., chloride, bromide, or iodide) reagent in ethereal solvent such as diethyl ether or tetrahydrofuran, at room temperature to 70° C. The coupling is facilitated by 2-20 mol % palladium catalyst. Appropriate catalysts include, for example dichlorobis(triphenylphosphine)palladium(II), dichlorobis(tri-o-tolylphosphine) palladium(II), and [bis(diphenylphosphino)ferrocene]palladium(II) (1:1 complex with CH₂Cl₂).

Suitable reduction reagents for step 6 include, for example, borane-dimethylsulfide complex, borane-tetrahydrofuran complex, borane-dimethylamine complex, lithium aluminum hydride, and hydrogen gas over palladium on carbon.

Suitable acids for use in deprotection (see steps 8 and 9) include, for example, trifluoroacetic acid in CH₂Cl₂ and 4 N HCl in ether or dioxane. Suitable acetylating reagents include acetylimidazole, diacetylmethoxylamine (Kikugawa, Y. et al. Tetrahedron Lett., 1990, 31, 243-246), and acetic acid /1-hydroxybenzotriazole /1-(3-dimethylaminopropyl)-3-ethylcarbodiimide.

### Example 4

### PREPARATION OF (1S,2R)N-[3-[3-BROMO-5-(2,2-DIMETHYL-PROPYL)-BENZYLAMINO]-1-(3,5-DIFLUORO-BENZYL)-2-HYDROXY-PROPYL]-ACETAMIDE

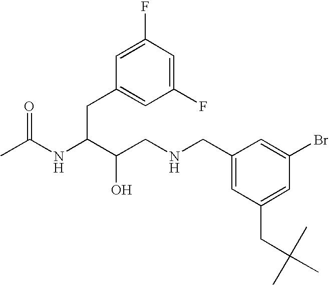

Dibromobenzylamine (1S,2R)N-[3-(2,5-Dibromo-benzylamino)-1-(3,5-difluoro-benzyl)-2-hydroxy-propyl]-acetamide (0.504 g, 1.0 mM, 1 eq) was added to 0.5 M THF solution of neopentylzinc iodide (20 mL, 10 eq) and [1,1′-bis(diphenylphosphino)ferrocene] dichloropalladium(II) complex (0.082 g, (0.1 mM, 0.1 eq)) with CH₂Cl₂ (Pd(dppf)Cl₂ CH₂Cl₂). A reaction mixture was stirred overnight at room temperature then quenched with saturated aqueous NH₄Cl (20 mL) and extracted with ethyl acetate. Combined organic layers were washed with brine, dried and concentrated.

The resulting solid was purified by HPLC, yielding (1S,2R)N-[3-[3-bromo-5-(2,2-dimethyl-propyl)-benzylamino]-1-(3,5-difluoro-benzyl)-2-hydroxy-propyl]-acetamide (0.055 g (11%)). ¹H NMR (300 MHz, DMSO-d₆) δ 8.60-9.00 (m, 1H), 7.88 (d, J=8.7 Hz, 1H), 7.61 (s, 1H), 7.39 (s, 1H), 7.08 (s, 1H), 7.05 (t, J=7.5 Hz, 1H), 6.93 (d, J=6.9 Hz, 2H), 4.16 (bs, 2H), 3.85 (m,1H), 3.70 (m, 1H), 3.02 (m, 2H), 2.81 (m, 1H), 2.57 (m, 1H), 2.47 (s, 2H), 1.69 (s, 3H), 0.87 (s, 9H); ¹³C NMR (300 MHz, DMSO-d₆) δ 170.0, 164.4, 164.2, 161.1, 160.9, 144.2, 142.9, 134.2, 133.9, 131.8, 131.0, 121.6, 112.9, 112.6,102.2, 69.3, 53.5, 50.1, 49.1, 35.4, 32.1, 29.6, 23.0; Cl MS m/z 497.2 [M+H]⁺.

### Examples 5-34

### GENERAL PROCEDURES FOR BIARYL PRECURSORS AND SYNTHESIS OF BIARYL COMPOUNDS

Additionally, where appropriate and unless otherwise noted, reactions were monitored, and purity evaluated by TLC on silica gel GF, 250μ slides obtained from Analtech, Inc., Newark, Del. Preparative low pressure (flash) chromatography was carried out on silica gel 60 (230-400 mesh ASTM) from EM Science. Proton NMR spectra were collected on a Bruker Avance 400 spectrometer. Chemical shifts (6) are in ppm, coupling constants (J) are in Hz. IR absorbances greater than 1200 cm⁻¹ are reported. All reagents were obtained from commercial sources and were used without further purification. Unless otherwise noted, all solvents used in reaction were run under N₂(g) in oven-dried glassware. HPLC analysis was carried out on a HP1100 system (Agilent) with the following a 1.0 mL/min linear gradient of 0.05% aqueous TFA (A) and 0.05% TFA in acetonitrile (B), 0% B: 5 min: 60% B, 15 min: 90% B, 2 min: 0% B. All solvents for chromatography were HPLC grade. Where not commercially available, starting materials and intermediates, including new and known compounds, were prepared by synthetic methods known in the art. HATU, which stands for N-[(dimethylamino)-1-H-1,2,3-triazolo[4,5-b]pyrindin-1-ylmethylene]-N-methylmethanaminium hexafluorophosphate N-oxide, was bought from PE Biosystems. All hydrochloride salts were formed by addition of ethereal HCl to an ethereal solution of amine, followed by concentration to dryness.

### Example 5

### PREPARATION OF N-1-(3,5-DIFLUOROBENZYL)-2-HYDROXY-3-{[(4-ISOBUTYL-1,1′-BI PHENYL-2-YL)METHYL]AMINO}PROPYL) ACETAMIDE

### STEP 1. 5-bromo-2-hydroxybenzamide

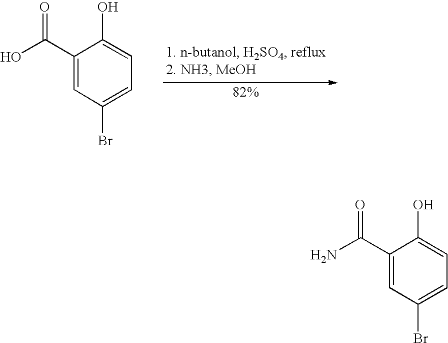

H₂SO₄ (95.6%, 289 μL, 5.42 mmol) was added to a stirred solution of 5-bromosalicyclic acid (30 g, 135.5 mmol) in n-butylalcohol (60 mL) in a 100 mL round bottom flask connected by a Dean-Stark trap/reflux condenser that was filled with 12 mL of n-butylalcohol. The reaction was refluxed for two days, then cooled to room temperature and concentrated to produce a pale yellow oil. MeOH (50 mL) was added to the mixture, followed by NH₃ in MeOH (7 N, 116 mL). The reaction was stirred at room temperature for another two days, then concentrated to yield a white solid. The crude solid was washed with small amount of ethyl acetate and hexane to yield the product as a white crystalline solid (24 g, 82%). ¹H NMR (CDCl₃) δ 12.15 (s, 1H), 7.54 (m, 2H), 6.97 (d, J=12 Hz, 1H), 6.00 (broad, 2H).

### STEP 2. Preparation of 2-hydroxy-5-isobutylbenzamide

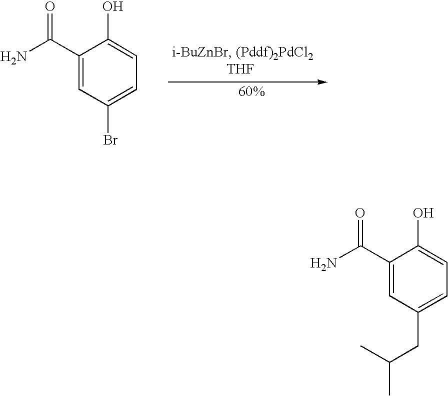

[1,1′-Bis(diphenylphosphino)ferrocene]dichloropalladium(II) (1.96 g, 2.4 mmol) was added to a stirred solution of the bromobenzamide (8.64 g, 40 mmol) in THF (100 mL) under argon followed by i-BuZnBr (0.5 M, 200 mL). The reaction was stirred at room temperature for four days then quenched with 1N HCl, and then concentrated. The resulting crude was diluted with ethyl acetate, and washed with water and brine, dried with sodium sulfate, filtered, and concentrated under reduced pressure. The crude product was purified by flash column chromatography (5˜10% ethyl acetate:hexane) to yield the isobutylbenzamide product as an off-white solid (4.63 g, 60% yield). ¹H NMR (CDCl₃) δ 12.02 (s, 1H), 7.24 (d, J=8 Hz, 1H), 7.12 (s, 1H), 6.93 (d, J=8 Hz, 1H), 2.44 (d, J=8 Hz, 2H), 1.83 (m, 1H), 0.93 (d, J=8 Hz, 6H).

### STEP 3. Preparation of 2-cyano-4-isobutylphenyl trifluoromethanesulfonate

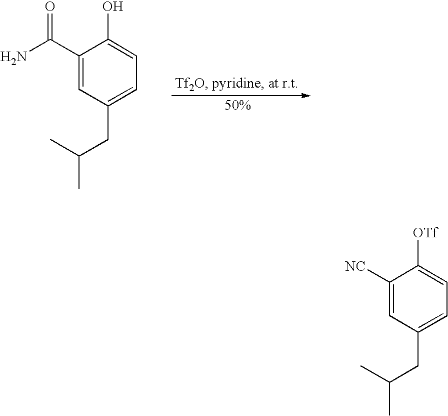

At 0° C., trifluoromethanesulfonic anhydride (10.2 mL, 57.8 mmol) was added to a stirred solution of the hydroxy-isobutylbenzamide (3.72 g, 19.3 mmol) in pyridine (15 mL) under argon. The reaction mixture was heated to room temperature and stirred overnight. The reaction was diluted with ethyl acetate, and washed with 1N HCl, water and brine, dried (sodium sulfate), filtered, and concentrated under reduced pressure. The crude product was purified by flash column chromatography (5% ethyl acetate:hexane) to yield the desired product as a clear oil (2.66 g, 50% yield). ¹H NMR (CDCl₃) δ 7.56 (s, 1H), 7.50 (d, J=8 Hz, 1H), 7.43 (d, J=8 Hz, 1H), 2.57 (d, J=8 Hz, 2H), 1.92 (m, 1H), 0.97 (d, J=4 Hz, 6H).

### STEP 4. Preparation of 4-isobutyl-1,1′-biphenyl-2-carbonitrile

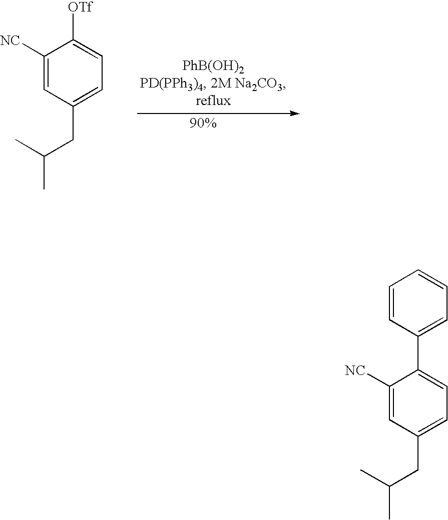

Tetrakis(triphenylphosphine) palladium(0) (109 mg, 0.094 mmol) was added to a stirred solution of the cyano compound (610 mg, 1.88 mmol), aqueous sodium carbonate (2.0 M, 3.76 mmol) in DME (6 mL) followed by phenylboronic acid (280 mg, 2.26 mmol). The reaction was heated to reflux overnight, and then cooled to room temperature. The reaction was diluted with ethyl acetate, and was washed with water and brine, dried (sodium sulfate), filtered, and concentrated under reduced pressure. The crude product was purified by flash column chromatography (3% ethyl acetate:hexane) to yield 450 mg of the product as a white solid (90% yield). ¹H NMR (CDCl₃) δ 7.60 (m, 3H), 7.54 (m, 2H), 7.48 (m, 3H), 2.60 (d, J=8 Hz, 2H), 1.96 (m, 1H), 1.00 (d, J=6 Hz, 6H).

### STEP 5. Preparation of (4-isobutyl-1,1′-biphenyl-2-yl)methylamine

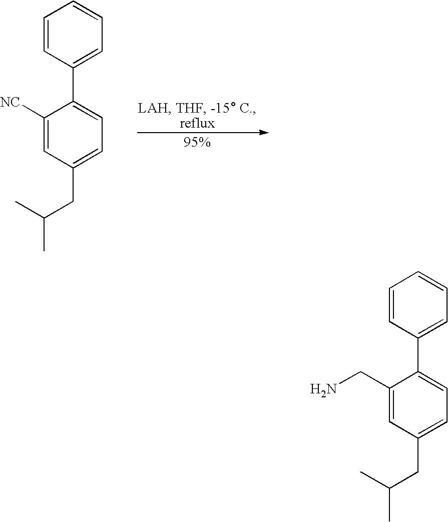

The above compound was prepared essentially according for the method of preparing 3-ethyl-α-propylbenzyl amine from 3-ethyl-α-propylbenzyl azide: 3-ethyl-α-propylbenzyl azide (724 mg, 3.57 mmol) in dry THF (10 mL) was added to a suspension of lithium aluminum hydride (280 mg, 7.38 mmol) in THF (10 mL) at 0° C. This was stirred at 0° C. for 30 min, then at room temperature for 1 h, whereupon the reaction was quenched using water (0.2 mL), 15% aq. NaOH (0.2 mL), and water (0.6 mL) in succession. This was stirred at room temperature for 1 h. The reaction mixture was then filtered through diatomaceous earth (CH₂Cl₂ elution), and the filtrate concentrated under reduced pressure. This material was used in subsequent reactions without further purification. ¹H NMR (CDCl₃) δ 7.47 (m, 2H), 7.44 (m, 3H), 7.30 (s, 1H), 7.20 (d, J=8 Hz, 1H), 7.14 (m, 1H), 3.84 (s, 2H), 2.58 (d, J=8 Hz, 2H), 1.93 (m, 1H), 1.47 (s, 2H), 1.00 (d, J=4 Hz, 6H); ESI-MS m/z 240.22 [M+H⁺]⁺.

### STEP 6. Preparation of tert-butyl (1S,2R)-1-(3,5-difluorobenzyl)-2-hydroxy-3-{[(4-isobutyl-1,1′-biphenyl-2-yl)methyl]amino}propylcarbamate

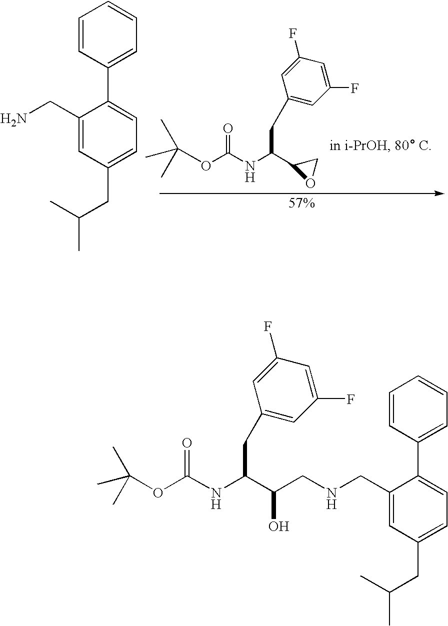

Tert-butyl (1S)-2-(3,5-difluorophenyl)-1-[(2S)-oxiran-2-yl]ethylcarbamate (336 mg, 1.12 mmol) was added to a stirred solution of the biphenyl amine (400 mg, 1.67 mmol) in isopropanol (10 mL). The reaction mixture was heated at 80° C. overnight. The reaction mixture was concentrated, and purified by flash column chromatography (2-5% MeOH: CH₂Cl₂) to yield of the product as an off-white solid (510 mg, 57% yield). ¹H NMR (CDCl₃) δ 7.45 (m, 2H), 7.38 (m, 3H), 7.25 (s, 1H), 7.21 (m, 1H), 7.16 (m, 1H), 6.76 (m, 2H), 6.70 (m, 1H), 4.55 (m, 1H), 3.76 (m, 3H), 3.34 (m, 1H), 2.90 (m, 1 H), 2.78 (m, 2H), 2.64 (m, 2H), 2.55 (m, 3H), 1.93 (m, 1H), 1.40 (s, 9H), 1.00 (d, 6H); ESI-MS m/z 539.22 [M+H⁺]⁺.

### STEP 7. Preparation of N-((1S,2R)-1-(3,5-Difluorobenzyl)-2-hydroxy-3-{[(4-isobutyl-1,1′-biphenyl-2-yl)methyl]amino}propyl) acetamide

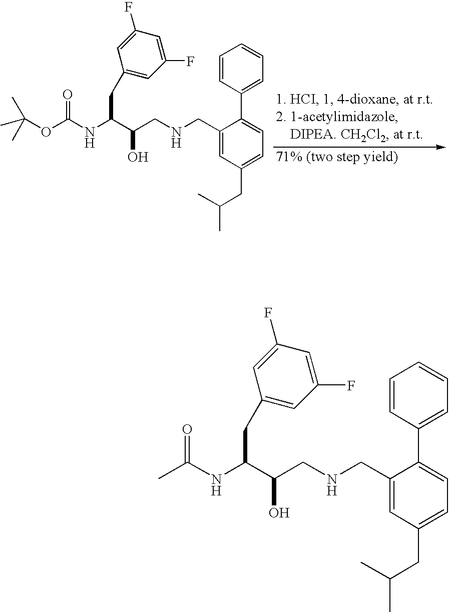

### STEP 7a

HCl in 1,4-dioxane (4.0 M, 2 mL) was added to a stirred solution of the starting material (377 mg, 0.7 mmol) in MeOH (5 mL). After stirring at room temperature overnight, the reaction mixture was concentrated under reduced pressure to yield an off-white solid, which was used without further purification.

### STEP 7b

DIPEA (304 μL, 1.75 mmol) was added to a stirred solution of amine from step 1 in CH₂Cl₂ (8 mL), followed by addition of 1-acetylimidazole (86 mg, 0.77 mmol). The reaction mixture was stirred at room temperature overnight, quenched by addition of 50% ammonium hydroxide, and diluted with CH₂Cl₂. The organic layer was washed with 1N HCl (×2), saturated aqueous sodium bicarbonate (×2) and brine (×1), dried with sodium sulfate, filtered, and concentrated under reduced pressure. The crude product was purified by flash column chromatography (3-5% MeOH: CH₂Cl₂) to yield 240 mg of product as an off-white solid (71% yield, two steps). ¹H NMR (CDCl₃) δ 9.63 (b, 1H), 8.48 (b, 1H), 7.63 (s, 1H), 7.46 (m, 3H), 7.28 (m, 4H), 6.74 (m, 2H), 6.67 (m, 1H), 4.24 (m, 1H), 4.17 (m, 1H), 4.05 (m, 2H), 2.80 (m, 4H), 2.57 (m, 3H), 1.97 (m, 4H), 0.97 (d, 6H); ESI-MS [M+H⁺]⁺=481.35.

### Example 6

### PREPARATION OF N-(1-(3,5-DIFLUOROBENZYL)-2-HYDROXY-3-{[2-(1H-IMIDAZOL-1-YL)-5-ISOBUTYLBENZYL]AMINO}PROPYL) ACETAMIDE

### STEP 1. Preparation of 2-(1H-imidazol-1-yl)-5-isobutyl benzonitrile

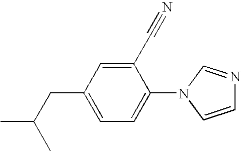

The above compound was prepared essentially according to the method described below in Example 5, step 2 and Examples 27 and 28. The resulting crude product was purified by flash column chromatography (50-100% o ethyl acetate:hexane) yielding the product as a dark-brown oil. ¹H NMR (CDCl₃) δ 7.89 (s, 1H), 7.60 (s, 1H), 7.53 (d, J=8 Hz, 1H), 7.40 (m, 2H), 7.28 (m, 1H), 2.60 (d, J=8 Hz, 2H), 1.93 (m, 1H), 0.97 (d, 6H); ESI MS m/z 226.03 [M+H⁺]⁺.

### STEP 2. Preparation of tert-butyl-(1S,2R)-1-(3,5-difluorobenzyl)-2-hydroxy-3-{[2-(1 h-imidazol-1-yl)-5-isobutylbenzyl]amino} propylcarbamate

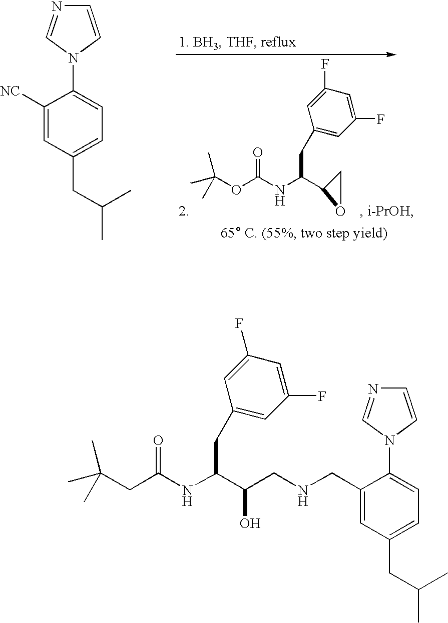

### STEP 2a

At 0° C., the imidazolyl product from step 1 (722 mg, 3.2 mmol) in anhydrous THF (8 mL) was added to a stirred solution of BH₃ (1.5 M in THF, 4.9 mL). The reaction was heated to room temperature, then refluxed for overnight. The reaction was cooled to room temperature and quenched with 5N aqueous HCl. The reaction was poured into CH₂Cl₂, washed with saturated aqueous sodium bicarbonate and brine, dried (sodium sulfate), filtered, and concentrated under reduced pressure. The product was used without further purification.

### STEP 2b

(1S)-2-(3,5-difluorophenyl)-1-[(2S)-oxiran-2-yl]ethylcarbamate (509 mg, 1.7 mmol) was added to a stirred solution of amine from step 2a in isopropanol (14 mL). The reaction mixture was heated at 65° C. overnight. The reaction mixture was concentrated, and purified by flash column chromatography (5-20% MeOH. CH₂Cl₂) to yield the product as an off-white solid (537 mg, 55% yield, two steps). ESI-MS m/z 529.35 [M+H⁺]⁺.

### STEP 3. Preparation of n-((1S,2R)-1-(3,5-difluorobenzyl)-2-hydroxy-3-{[2-(1 h-imidazol-1-yl)-5-isobutylbenzyl]amino}propyl) acetamide

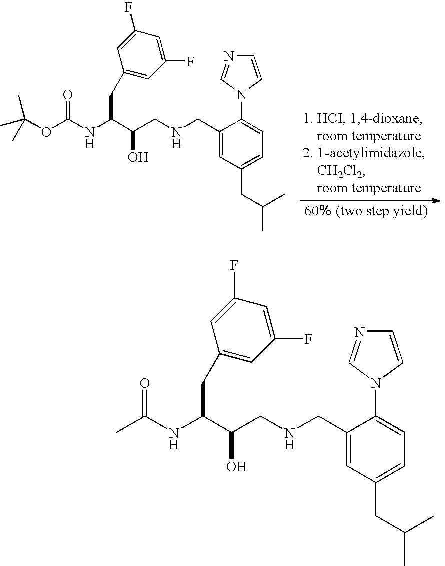

The above compound was prepared essentially according to the method of Example 5, step 7. The crude acetamide was purified by flash column chromatography (5-20% MeOH: CH₂Cl₂) to yield the desired product as an off-white solid (60% yield, two steps). ESI-MS m/z 471.33 [M+H⁺]⁺.

### Example 7

### PREPARATION OF N-((1S,2R)-1-(3,5-DIFLUOROBENZYL)-2-HYDROXY-3-{[5-ISOBUTYL-2-(1H-1,2,4-TRIAZOL-1-YL)BENZYL]AMINO}PROPYL)ACETAMIDE

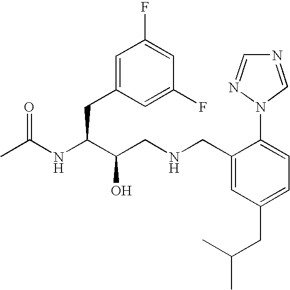

The above compound is synthesized using procedures essentially similar to Example 6, steps 2 and 3. ESI MS m/z 472.0 [M+H⁺]⁺.

### Example 8

### PREPARATION OF N-1-(3,5-DIFLUOROBENZYL)-3-{[(3′-FLUORO-4-ISOBUTYL-1,1′-BIPHENYL-2-YL)METHYL]AMINO}-2-HYDROXYPROPYL)ACETAMIDE

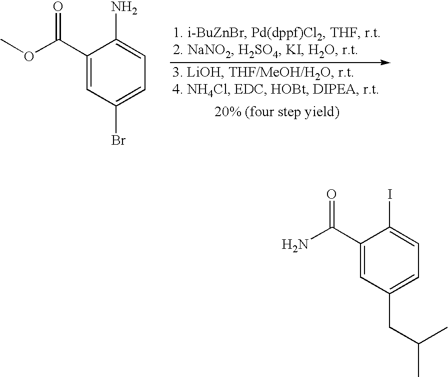

### STEP 1a: Preparation of 2-iodo-5-isobutylbenzamide

[1,1′-Bis(diphenylphosphino)ferrocene] dichloropalladium(II) (2.04 g, 2.5 mmol) followed by i-BuZnBr (0.5 M, 200 mL) was added to a stirred solution of methyl 2-amino-5-bromobenzoate (5.77 g, 25 mmol) in THF (20 mL) under argon. The reaction mixture was stirred at room temperature overnight, then quenched with 1N HCl and concentrated. The resulting crude was diluted with ethyl acetate, and washed with water and brine, dried (sodium sulfate), filtered, concentrated, and used without further purification.

### STEP 1b

At room temperature the amine from step 1a was treated with 5% H₂SO₄ (3.2 mL), then heated to 60° C. for 5-10 min. The reaction mixture was cooled to 0° C., and then NaNO₂ (1.87 g, 27 mmol) in H₂O (10 mL) was added drop-wise. After the addition was complete, the reaction was stirred at ice-cold temperature for 15-20 min, and then KI (4.94 g, 29.7 mmol) in H₂O (20 mL) was added. The reaction was stirred at room temperature overnight. The reaction was extracted with ethyl acetate, washed with brine, dried (sodium sulfate), filtered, and concentrated. The crude product was purified by flash column chromatography (5-10% MeOH: CH₂Cl₂) to yield of iodinated product (2 g).

### STEP 1c

LiOH.H₂O (4.4 mg, 104.5 mmol) was added to a stirred solution of iodinated product from step 1b (6.6 g, 20.9 mmol) in a mixed solvent of MeOH (30 mL), THF (30 mL), and water (30 mL) at room temperature. After stirring for 12 h at room temperature, the reaction mixture was quenched with 1N HCl, diluted with CH₂Cl₂, washed with saturated aqueous sodium bicarbonate, water, and brine, dried (sodium sulfate), and concentrated under reduced pressure. The crude product was used without further purification.

### STEP 1d

3-Amino-4-(3,5-difluorophenyl)-1-([(4S)-6-iodo-3,4-dihydro-2H-chromen-4-yl]amino}butan-2-ol (1 equiv) was combined with 2-methylacetic acid, (1.25 equiv), EDC (1.5 equiv) and HOBt (1.5 equiv) in DMF/DCM (1:1, 10 mL). The reaction mixture was treated with Et₃N and stirred for 6 h. The reaction mixture was then poured into ethyl acetate and washed with 1M HCl, dried (magnesium sulfate), and concentrated yielding an oil which was purified by reverse phase preparative HPLC, then purified by flash column chromatography (10-50% EtOAC: CH₂Cl₂) to yield the product as an off-white solid (900 mg, 20% yield, four steps). ¹H NMR (CDCl₃) δ 7.80 (d, J=8 Hz, 1H), 7.30 (s, 1H), 6.95 (d, J=8 Hz, H), 5.80 (b, 2H), 2.47 (d, J=6 Hz, 2H), 1.87 (m, 1H), 0.93 (2, H).

### STEP 2. Preparation of N-{1-(3,5-difluorobenzyl)-2-hydroxy-3-[(2-iodo-5-isobutylbenzyl)amino] propyl}acetamide

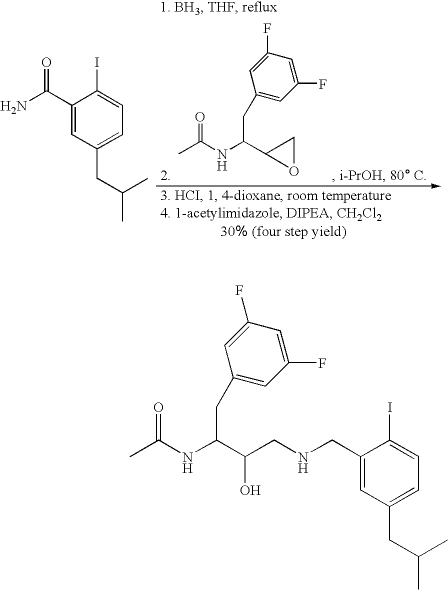

### STEP 2a

At 0° C., 2-Iodo-5-isobutyl-benzamide (1.838 g, 6.1 mmol) was added to a stirred solution of BH₃ (1.5 M in THF, 9.3 mL) in anhydrous THF (16 mL). The reaction was heated to room temperature, refluxed overnight, cooled to room temperature, and then quenched with 5N aqueous HCl. The reaction was added to CH₂Cl₂ (10 mL), washed with saturated aqueous sodium bicarbonate and brine, dried (sodium sulfate), filtered, concentrated under reduced pressure, and used without further purification.

### STEP 2b

An isopropyl alcohol (IPA) solution of tert-butyl (1S)-2-(3,5-difluorophenyl)-1-[(2S)-oxiran-2-yl]ethylcarbamate and 6-Iodo-chroman-4-ylamine was stirred at 75° C. overnight. The IPA was removed in vacuo and the resulting residue dissolved in EtOAc (200 mL). The organic layer was washed with 1 N HCl (4×50 mL), followed by NaHCO₃, and brine. The organic layer was dried (sodium sulfate) and concentrated in vacuo to yield a mixture of diastereomers. The reaction mixture was used without further purification.

### STEP 2c

The product of step 2b in a stirred solution in MeOH (10 mL) was added to HCl in 1,4-dioxane (4.0 M, 5.6 mL). After stirring at room temperature overnight, the reaction mixture was concentrated under reduced pressure. The crude product was re-dissolved in CH₂Cl₂, washed with saturated aqueous sodium bicarbonate and brine, dried (sodium sulfate), filtered, concentrated under reduced pressure, and used without further purification.

### STEP 2d

DIPEA (3.88 mL, 22.3 mmol), and then 1-acetylimidazole (516 mg, 4.46 mmol), were added to a stirred solution of amine from step 2c in CH₂Cl₂ (60 mL). The reaction mixture was stirred at room temperature overnight, quenched by addition of 50% ammonium hydroxide, and diluted with CH₂Cl₂. The organic layer was washed with 1N HCl, saturated aqueous sodium bicarbonate and brine, dried with sodium sulfate, filtered, and concentrated under reduced pressure. The crude product was purified by flash column chromatography (3-5% MeOH: CH₂Cl₂) to yield the product as an off-white solid (1 mg, 30% yield, four steps). ¹H NMR (CDCl₃) δ 7.76 (d, J=9 Hz, 1H), 7.14 (s, 1H), 6.76 (m, 4H), 5.97 (d, J=3 Hz, 1H), 4.20 (m, 1H), 3.84 (m, 2H), 3.63 (m, 1H), 2.81 (m, 4H), 2.46 (d, J=6 Hz, 2H), 1.88 (m, 4H), 0.92 (d, 6H).

### STEP 3. Preparation of N-((1S,2R)-1-(3,5-difluorobenzyl)-3-{[(3′-fluoro-4-isobutyl-1,1′-biphenyl-2-yl)methyl]amino}-2-hydroxypropyl)acetamide

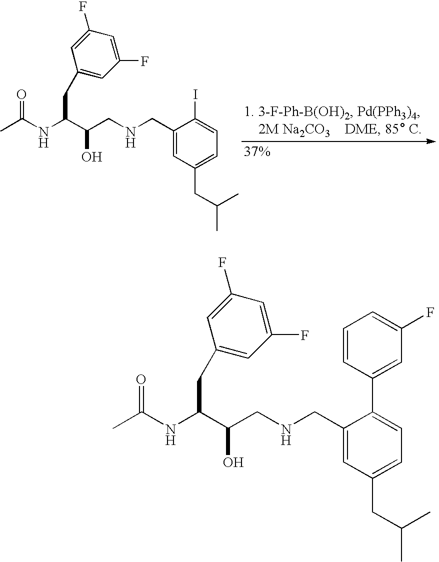

Tetrakis(triphenylphosphine) palladium(0) (21 mg, 0.0183 mmol) was added to a stirred solution of the product of step 2 (97 mg, 0.183 mmol), aqueous sodium carbonate (2.0 M, 0.403 mmol) in DME (1 mL) followed by addition of 3-fluoro-phenylboronic acid (64 mg, 0.458 mmol). The reaction mixture was heated to reflux overnight, cooled to room temperature, diluted with CH₂Cl₂, washed with water and brine, dried (sodium sulfate), filtered, and concentrated under reduced pressure. The crude product was purified by flash column chromatography (3-10% MeOH: CH₂Cl₂) to yield the product as a white solid (36 mg, 37% yield). ESI MS m/z 499.32 [M+H⁺]⁺.

### Example 9

### PREPARATION OF N-[(1S,2R)-3-(3,4-DIBROMOBENZYL AMINO)-1-(3,5-DIFLUOROBENZYL)-2-HYDROXY-PROPYL]-ACETAMIDE

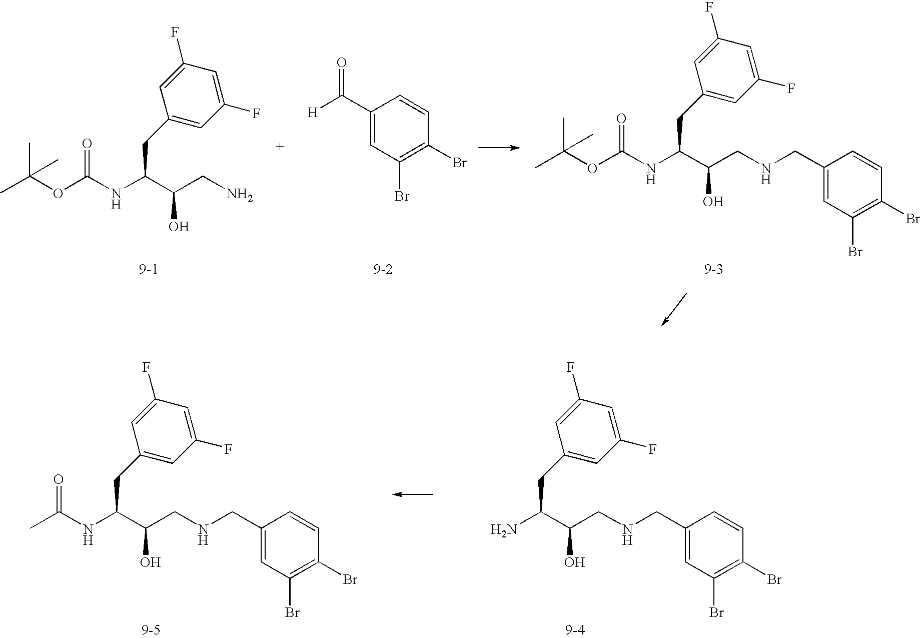

### STEP 1. Preparation of [(1S,2R)-3-(3,4-dibromobenzylamino)-1-(3,5-difluorobenzyl)-2-hydroxypropyl] carbamic acid tert-butyl ester

Commercially available 3,4-dibromobenzaldehyde (250 mg, 0.95 mmol) and N-Boc-diamine 9-1 (250 mg, 0.79 mmol) were dissolved together in 10% acetic acid in THF (10 mL). After the solution was allowed to stand at room temperature for 30 min., 1.7 g (˜3.8 mmol) of MP-cyanoborohydride (a macroporous triethylammonium methylpolystyrene cyanoborohydride, Argonaut Corporation) was added. The suspension was agitated for 3 h, then filtered, and concentrated under reduced pressure. The residue was dissolved in methanol and purified by reversed phase HPLC. Fractions containing pure compound 9-3 were concentrated under reduced pressure. MS m/z 564.7 [M+H]⁺.

### STEP 2. Preparation of (3S,2R)-3-amino-1-(3,4-dibromo-benzylamino)-4-(3,5-difluorophenyl)-butan-2-ol

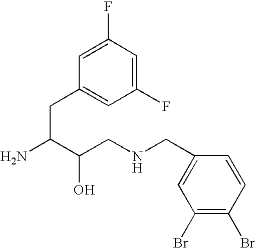

Trifluoroacetic acid (anhydrous) was added to a solution of compound 9-3 in anhydrous CH₂Cl₂. The solution stood for 90 min then the volatiles are removed with a stream of N₂(g). The compound was dissolved in ethyl acetate and washed with saturated aqueous sodium bicarbonate. The organic phase is then dried (magnesium sulfate (anh.)), filtered, and concentrated yielding (3S,2R)-3-amino-1-(3,4-dibromo-benzylamino)-4-(3,5-difluorophenyl)-butan-2-ol. MS m/z 464.8[M+H]⁺.

### STEP 3. Preparation of N-[(1S,2R)-3-(3,4-dibromobenzyl amino)-1-(3,5-difluorobenzyl)-2-hydroxy-propyl]-acetamide

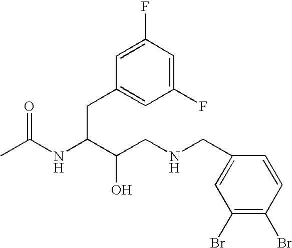

HOBt, N-methyl-morpholine, and glacial acetic acid were added to a solution of compound 9-4 in anhydrous CH₂Cl₂. This solution is cooled to 0° C. and then solid EDC-HCl (1-[3-(dimethylamino)propyl]-3-ethylcarbodiimide hydrochloride and a stir bar were added. The reaction was stirred at 0° C. for 12 h. After warming to room temperature, the solvent was removed with a stream of N₂(g). The residue was dissolved in ethyl acetate and washed with aqueous saturated sodium bicarbonate. The ethyl acetate phase is dried (magnesium sulfate), filtered, then concentrated under reduced pressure to yield N-[(1S,2R)-3-(3,4-dibromobenzyl amino)-1-(3,5-difluorobenzyl)-2-hydroxy-propyl]-acetamide. MS m/z 506.8 [M+H]⁺.

### Example 10

### PREPARATION OF N-[(1S,2R)-3-{[(4-BROMO-1,1′-BIPHENYL-2-YL)METHYL]AMINO}-1-(3,5-DIFLUOROBENZYL)-2-HYDROXYPROPYL] ACETAMIDE

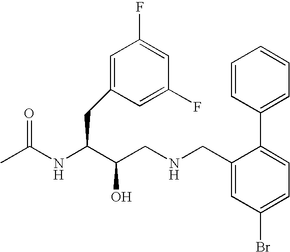

### STEP 1. Preparation of 5-Bromo-2-iodobenzamide

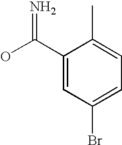

HATU (25 g, 65.8 mmol) was added to 5-bromo-2-iodobenzoic acid (20 g, 61.2 mmol) in 1:1 mixture of CH₂Cl₂ and dimethylformamide (200 mL) and the solution stirred 2 min. Ammonium chloride (20 g) was added, and the heterogeneous mixture was stirred 1 h. Ammonium hydroxide (20 mL) was added. The solution was filtered, diluted with ethyl acetate, washed with water, 1 N HCl, saturated sodium bicarbonate, and saturated NaCl, dried (magnesium sulfate), filtered, and concentrated under reduced pressure to yield a white precipitate. The solid was filtered to provide 5-bromo-2-iodobenzamide (14.4 g). ESI MS m/z 327.0 [M+H]⁺.

### STEP 2. Preparation of (4-Bromo-1,1′-biphenyl-2-yl)methylamine

Palladium(0) tetrakis(triphenylphosphine) (2.6 g, 2.2 mmol) was added to a stirred solution of 5-bromo-2-iodobenzamide (14.1 g, 43.3 mmol), phenyl boronic acid (5.3 g, 43.3 mmol), and potassium carbonate (24.4 g, 176.8 mmol) in degassed dimethylformamide (100 mL). The reaction was refluxed overnight under N₂(g). The brown solution was cooled and filtered through Celite. The solution was diluted in ethyl acetate, washed with water, 1 N HCl, saturated sodium bicarbonate, and saturated NaCl, dried (magnesium sulfate), filtered, and concentrated under reduced pressure to a tar. Flash chromatography (silica, 50% ethyl acetate/hexane) gave a tan solid (2.4 g). The biphenyl amide was dissolved in tetrahydrofuran (20 mL), and BH₃-THF (1N, 20 mL, 20 mmol) was added slowly. The reaction was refluxed overnight under N₂. The reaction was cooled to 0° C. and quenched with ethyl acetate resulting in gas evolution. After gas evolution ceased, the organics were washed with water, saturated sodium bicarbonate, saturated NaCl, dried (sodium sulfate), filtered, and concentrated yielding (4-bromo-1,1′-biphenyl-2-yl)methylamine as a gray semi-solid (2.4 g). ESI MS m/z 262.0/264.0 [M+H]⁺.

### STEP 3. Preparation of N-[(1S,2R)-3-{[(4-Bromo-1,1′-biphenyl-2-yl)methyl]amino}-1-(3,5-difluorobenzyl)-2-hydroxypropyl] acetamide

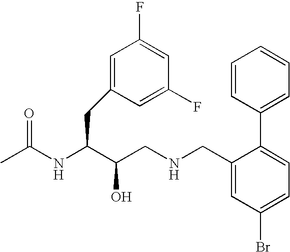

Tert-butyl (1S)-2-(3,5-difluorophenyl)-1-[(2S)-oxiran-2-yl]ethylcarbamate (1.8 g, 6.1 mmol) was added to solution of (4-bromo-1,1′-biphenyl-2-yl)methylamine (2.4 g, 9.2 mmol) in isopropanol (50 mL) and the reaction was refluxed 2 h. The solution was concentrated, and the residue was redissolved in ethyl acetate, washed with 1 N HCl and saturated NaCl, dried (sodium sulfate), filtered, and concentrated under reduced pressure. The residue (3.3 g) was dissolved in methanol, and 4 N HCl in dioxane (5 mL) was added. The reaction was stirred for 30 min, then concentrated to yield a tan foam (3.1 g). The salt was dissolved in CH₂Cl₂ (25 mL) and diisopropylethylamine (4 mL, 23 mmol), then acetylimidazole (636 mg, 5.8 mmol) was added. The reaction was stirred overnight at room temperature. The organics were washed with water, 1N HCl, saturated sodium bicarbonate, and saturated NaCl, dried (sodium sulfate), filtered, and concentrated under reduced pressure. Purification by flash chromatography (10% methanol/CH₂Cl₂) provided N-[(1S,2R)-3-{[(4-bromo-1,1′-biphenyl-2-yl)methyl]amino}-1-(3,5-difluorobenzyl)-2-hydroxypropyl] acetamide (550 mg). ESI MS m/z 504.3 [M+H]⁺. A small amount of the product was dissolved in ether, precipitated with excess 1 N HCl in ether, and concentrated to provide the mono-HCl salt.

### Example 11

### PREPARATION OF N-[(1S,2R)-3-{[(4-ACETYL-1,1′-BIPHENYL-2-YL)METHYL]AMINO}-1-(3,5-DIFLUOROBENZYL)-2-HYDROXYPROPYL] ACETAMIDE HYDROCHLORIDE

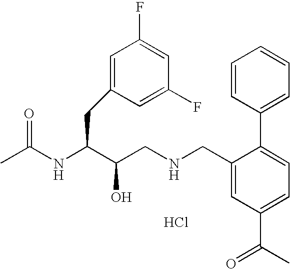

Tributyl(1-ethoxyvinyl)tin (100 μL, 0.28 mmol) and bis-triphenylphoshine palladium(II) dichloride (10 mg, 0.012 mmol) were added to N-[(1S,2R)-3-{[(4-bromo-1,1′-biphenyl-2-yl)methyl]amino}-1-(3,5-difluorobenzyl)-2-hydroxypropyl] acetamide (120 mg, 0.24 mmol) in toluene (1 mL), and the reaction was heated at 100° C. for 3 h under N₂. The solution was cooled to room temperature, 1 N HCl (1 mL) was added, and the mixture was stirred for 20 min. The mixture was partitioned and washed with saturated potassium fluoride (aq). The reaction mixture was dried (sodium sulfate), filtered, and concentrated under reduced pressure. Purification by flash column chromatography (silica gel, 8% methanol/CH₂Cl₂) yielded an oil. The residue was dissolved in ether, precipitated with 1N HCl in ether, yielding N-[(1S,2R)-3-{[(4-acetyl-1,1′-biphenyl-2-yl)methyl]amino}-1-(3,5-difluorobenzyl)-2-hydroxypropyl] acetamide hydrochloride (11 mg). ESI MS m/z 467.28 [M+H]⁺.

### Example 12

### PREPARATION OF N-[(1S,2R)-3-{[(4-SEC-BUTYL-1,1′-BIPHENYL-2-YL)METHYL]AMINO}-1-(3,5-DIFLUOROBENZYL)-2-HYDROXYPROPYL] ACETAMIDE

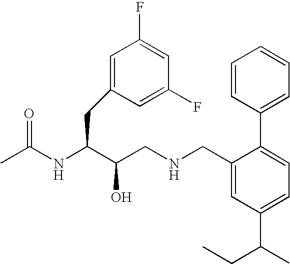

2M potassium phosphate (0.65 mmol), tri-sec butylborane (1M in THF, 330 μL, 0.33 mmol), and bis-triphenylphoshine palladium(II) dichloride (3 mg, 0.003 mmol) were added to N-[(1S,2R)-3-{[(4-bromo-1,1′-biphenyl-2-yl)methyl]amino}-1-(3,5difluorobenzyl)-2-hydroxypropyl] acetamide (150 mg, 0.3 mmol) in THF (2 mL), and the reaction was heated at reflux for 2 days. Tri-sec butylborane (1M in THF, 1.2 mL, 1.2 mmol) was added, then bis-triphenylphoshine palladium(II) dichloride (10 mg, 0.012 mmol), and the reaction was refluxed 16 h. The solution was diluted in ethyl acetate and washed with water, 1N HCl, saturated sodium bicarbonate, and saturated NaCl. The organic layer was dried over sodium sulfate, filtered and concentrated under reduced pressure. Flash chromatography (7% methanol/dichloromethane) yielded N-[(1S,2R)-3-{[(4-sec-butyl-1,1′-biphenyl-2-yl)methyl]amino)-1-(3,5-difluorobenzyl)-2-hydroxypropyl}acetamide. ESI MS m/z 481.34 [M+H⁺].

### Example 13

### PREPARATION OF N-(1-(3,5-DIFLUORO-BENZYL)-3-{[4-(2,2-DIMETHYL-PROPYL)-BIPHENYL-2-YLMETHYL]-AMINO}-2-HYDROXY-PROPYL)-ACETAMIDE

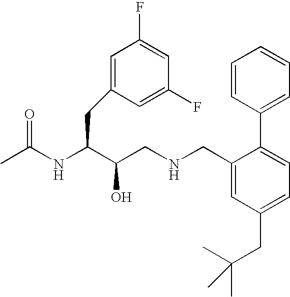

### STEP 1. Preparation of 4-Neopentyl-1,1′-biphenyl-2-carboxamide

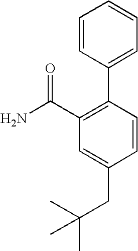

Palladium(0) tetrakis(triphenylphosphine) (751 mg, 0.65 mmol) was added to methyl 5-bromo-2-iodobenzoate (4.41 g, 13 mmol), phenylboronic acid (1.6 g, 13 mmol), potassium carbonate (3.6 g, 26 mmol), and cesium carbonate (4.2 g, 13 mmol) in degassed DMF (50 mL). The reaction was refluxed 16 h, cooled and washed with water, 1N HCl, saturated sodium bicarbonate, and saturated NaCl, dried (sodium sulfate), filtered, and concentrated under reduced pressure. The residue was purified by flash chromatography (5% ethyl acetate/hexane) to yield methyl 4-bromo-1,1′-biphenyl-2-carboxylate (1.3 g). 1M neopentyl magnesium chloride (5 mL, 5 mmol) was added to methyl 4-bromo-1,1′-biphenyl-2-carboxylate (500 mg, 1.72 mmol) and Pd(dppf)C₁₂-CH₂Cl₂ (70 mg, 0.086 mmol) in THF (5 mL) slowly at room temperature. The reaction was stirred overnight and then quenched with water. The reaction was diluted with ethyl acetate, and the resulting brown solid was filtered away. The solution was washed with water, 1N HCl, saturated sodium bicarbonate, and saturated NaCl, and dried (sodium sulfate), filtered, and concentrated under reduced pressure. The residue was purified by flash chromatography (1% ethyl acetate/hexane) to yield a yellow solid (200 mg).

The solid was redissolved in 2:1:1 THF/methanol/water (8 mL) and lithium hydroxide monohydrate (60 mg, 1.4 mmol) was added. The reaction was stirred for 6 days, and the solution was concentrated to dryness. (An additional 1.7 g 4-bromo-1,1′-biphenyl-2-carboxylate was used to prepare a combined total of 1.8 g residue from hydrolysis). The pooled lots were dissolved in DMF (10 mL), and diisopropylethylamine (3.7 mL, 21 mmol), HATU (4 g, 10.2 mmol), and ammonium chloride (5 g) were added. The reaction was stirred for 1 h. Ammonium hydroxide was added causing a white precipitate. The liquid was diluted in ethyl acetate, washed with water, 1 N HCl, saturated sodium bicarbonate, and saturated NaCl, then dried (sodium sulfate), filtered and concentrated under reduced pressure to yield a black oil. The residue was purified by flash chromatography (60% ethyl acetate/hexane) to yield 4-neopentyl-1,1′-biphenyl-2-carboxamide as a tan solid (210 mg). ESI MS m/z 268 [M+H]⁺.

### STEP 2. Preparation of (4-Neopentyl-1,1′-biphenyl-2-yl)methylamine

4-Neopentyl-1,1′-biphenyl-2-carboxamide (200 mg, 0.75 mmol) was added to borane-THF (1M, 1.7 mL, 1.7 mmol), and the reaction was stirred at reflux for 16 h. The solution was cooled and quenched with 1N HCl. The solution was basified with saturated sodium bicarbonate, and the product was extracted into ethyl acetate. The organics were washed with saturated NaCl, dried (sodium sulfate), filtered, and concentrated under reduced pressure yielding (4-neopentyl-1,1′-biphenyl-2-yl)methylamine as an oil (200 mg). ESI MS m/z 254.22 [M+H]⁺.

### STEP 3. Preparation of N-((1S,2R)-1-(3,5-Difluorobenzyl)-2-hydroxy-3-{[(4-neopentyl-1,1′-biphenyl-2-yl)methyl]amino}propyl) acetamide hydrochloride

Tert-butyl (1S)-2-(3,5-difluorophenyl)-1-[(2S)-oxiran-2-yl]ethylcarbamate (120 mg, 0.4 mmol) was added to solution of (4-neopentyl-1,1′-biphenyl-2-yl)methylamine (200 mg, 0.8 mmol) in isopropanol (5 mL), and the reaction was refluxed 2 h. The solution was concentrated, dissolved in ethyl acetate, washed with 1 N HCl and saturated NaCl, dried (sodium sulfate), filtered, and concentrated under reduced pressure. The residue was dissolved in methanol, and 4 N HCl in dioxane (5 mL) was added. The reaction was stirred for 30 min, then concentrated to a white foam (100 mg). The salt was dissolved in CH₂Cl₂ (2 mL) and diisopropylethylamine (100 μL, 0.5 mmol), then acetylimidazole (30 mg, 0.3 mmol) was added. The reaction was stirred for 1 h at room temperature, then washed with water, 1 N HCl, saturated sodium bicarbonate, and saturated NaCl, dried (sodium sulfate), filtered, and concentrated under reduced pressure. Purification by flash chromatography (8% methanol/CH₂Cl₂) yielded N-((1S,2R)-1-(3,5-difluorobenzyl)-2-hydroxy-3-{[(4-neopentyl-1,1′-biphenyl-2-yl)methyl]amino}propyl) acetamide hydrochloride (60 mg) in crude form. The material was purified by preparative reversed phase HPLC yielding the desired compound. The product was dissolved in ether, precipitated with 1N HCl in ether, and concentrated to provide the mono-HCl salt (6 mg). ESI MS m/z 495 [M+H]⁺.

### Example 14

### PREPARATION OF N-[1-(3,5-DIFLUORO-BENZYL)-3-(2-FLUORO-5-ISOBUTYL-BENZYLAMINO)-2-HYDROXY-PROPYL]-ACETAMIDE

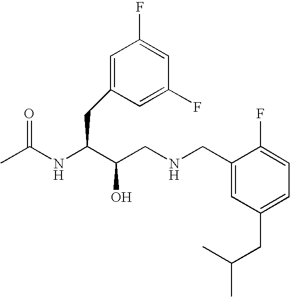

### STEP 1. Preparation of 2-Fluoro-5-isobutyl-benzonitrile

0.5 M isobutylzinc bromide in THF (70 mL, 35 mmol), then Pd(dppf)Cl₂ (955 mg, 1.17 mmol), were added to 5-bromo-2-fluorobenzonitrile (2.3 g, 11.7 mmol) in THF (5 mL), and the reaction was stirred 16 h at room temperature under N₂. The reaction was quenched with excess aqueous HCl (1N). Ethyl acetate was added, and the solution was washed with saturated NaCl. Flash chromatography (4% ethyl acetate/hexane) yielded a colorless oil (1.3 g).

### STEP 2. Preparation of N-[1-(3,5,-difluoro-benzyl)-3-(2-fluoro-5-isobutyl-benzylamino)-2-hydroxy-propyl]-acetamide

Borane-THF (1 M, 3 mL, 3 mmol) was added to the product from step 1 (230 mg, 1.3 mmol) in THF (2 mL) slowly at 0° C. The reaction was stirred 16 h at room temperature. The solution was cooled and quenched with 1N HCl. The solution was basified with saturated sodium bicarbonate, and the product was extracted into ethyl acetate. The organic layer was washed with saturated NaCl, dried (sodium sulfate), filtered, and concentrated under reduced pressure yielding an oil. The residue was dissolved in isopropanol (2 mL), tert-butyl (1S)-2-(3,5-difluorophenyl)-1-[(2S)-oxiran-2-yl]ethylcarbamate (120 mg, 0.4 mmol) was added, and the reaction was refluxed 3 h. 4N HCl in dioxane (5 mL) was added, and the reaction was stirred for 1.5 h, then concentrated to a white foam. The residue was dissolved in CH₂Cl₂ (5 mL) and diisopropylethylamine (678 μL, 3.9 mmol), then acetylimidazole (66 mg, 0.6 mmol) was added. The reaction was stirred for 30 min at room temperature. Additional acetylimidazole (30 mg, 0.3 mmol) was added. The organics were washed with water, saturated sodium bicarbonate, and saturated NaCl, dried (sodium sulfate), filtered, and concentrated under reduced pressure. Purification by flash chromatography (silica, 8% methanol/CH₂Cl₂) provided N-[1-(3,5-difluoro-benzyl)-3-(2-fluoro-5-isobutyl-benzylamino)-2-hydroxy-propyl]-acetamide as a white solid (89 mg). ESI MS m/z 423 [M+H]⁺.

### Example 15

### PREPARATION OF N-[(1S,2R)-1-(3,5-DIFLUOROBENZYL)-2-HYDROXY-3-({2-[(2-HYDROXYETHYL)AMINO]-5-ISOBUTYLBENZYL}AMINO)PROPYL]ACETAMIDE

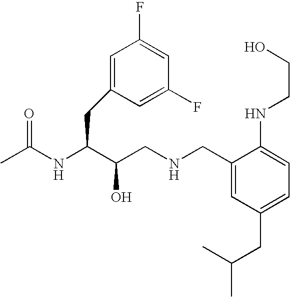

2-Fluoro-5-isobutyl-benzonitrile (0.533 g, 3 mmol) in ethanolamine (5 mL) was heated at 100° C. for 2 h in a sealed tube. The reaction was diluted with ethyl acetate, and the organic layer was washed with water and saturated NaCl, dried (sodium sulfate), filtered, and concentrated to an oil. The residue was dissolved in THF (3 mL) and added to borane-THF (9 mL) at 0° C. The reaction was stirred at room temperature for 16 h. The solution was poured onto ice, and ethyl acetate was added. The organics were washed with saturated NaCl, dried (sodium sulfate), filtered, and concentrated to an oil (220 mg).

The residue was dissolved in isopropanol (5 mL), tert-butyl (1S)-2-(3,5-difluorophenyl)-1-[(2S)-oxiran-2-yl]ethylcarbamate (160 mg, 0.5 mmol) was added, and the reaction was refluxed 2 h. The reaction was cooled and concentrated. Flash chromatography (silica, 8% methanol/CH₂Cl₂) yielded an oil (108 mg). The residue was treated with 4 N HCl in dioxane (5 mL). The reaction was stirred for 1 h, then concentrated to a white solid. The residue was dissolved in CH₂Cl₂ (5 mL) and diisopropylethylamine (108 μL, 0.6 mmol), then acetylimidazole (44 mg, 0.4 mmol) was added. The reaction was stirred for 30 min at room temperature. The organics were washed with water, saturated sodium bicarbonate, and saturated NaCl, dried (sodium sulfate), filtered, and concentrated under reduced pressure. Purification by flash chromatography (8% methanol/CH₂Cl₂) provided N-[(1S,2R)-1-(3,5-difluorobenzyl)-2-hydroxy-3-({2-[(2-hydroxyethyl)amino]-5-isobutylbenzyl}amino)propyl]acetamide as an oil (18 mg). ESI MS m/z 464.34 [M+H]⁺.

### Example 16

### PREPARATION OF N-{1-(3,5-DIFLUORO-BENZYL)-3-[5-(2,2-DIMETHYL-PROPYL)-2-(6-FLUORO-PYRIDIN-3-YL)-BENZYLAMINO]-2-HYDOXY-PROPYL}-ACETAMIDE

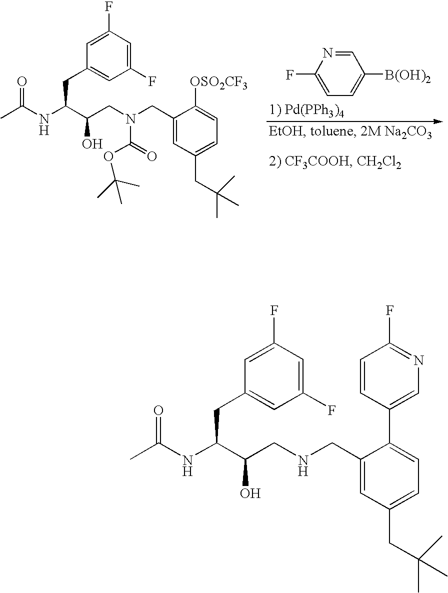

Trifluoro-methanesulfonic acid 2-({[3-acetylamino-4-(3,5-difluoro-phenyl)-2-hydroxyl-tert-butyoxycarbonyl-amino}-methyl)-4-(2,2-dimethyl-propyl)-phenyl ester (0.169 g, 0.253 mmol) was added to a flask followed by tetrakis(triphenyl)phosphine palladium(0) (0.018 g, 0.016 mmol), anh. toluene (0.5 mL) and 2.0 M sodium bicarbonate (0.5 mL). A solution of 2-fluoropyridine-5-boronic acid (0.051 g, 0.362 mmol) in EtOH (0.5 mL) was added to the stirred mixture. The reaction mixture was refluxed under N₂(g) overnight and then cooled to room temperature prior to partitioning between H₂O and Et₂O. The organic layer was separated, dried (sodium sulfate) and concentrated under reduced pressure. The residue was purified by flash chromatography (hexane/ethyl acetate, 1:1) yielding the coupled product (0.137 g, 88%): Cl MS m/z 514.2 [M+H-Boc]+, 636.3 [M+Na]⁺.

A solution of N-(1-(3,5-difluorophenyl)-4-(2-(6-fluoropyridin-3-yl)-5-neopentylbenzylamino)-3-hydroxybutan-2-yl)acetamide was stirred in a mixture of CH₂Cl₂ (1 mL) and CF₃COOH (1 mL) at room temperature for 2.5 h. The reaction mixture was partitioned between sat. NaHCO₃ (aq) and CH₂Cl₂. The organic layer was separated, dried (sodium sulfate), and concentrated under reduced pressure to yield the desired product. Cl MS m/z 514.2 [M+H]₊.

### Example 17

### SCHEME FOR HYDROXYL REPLACEMENT

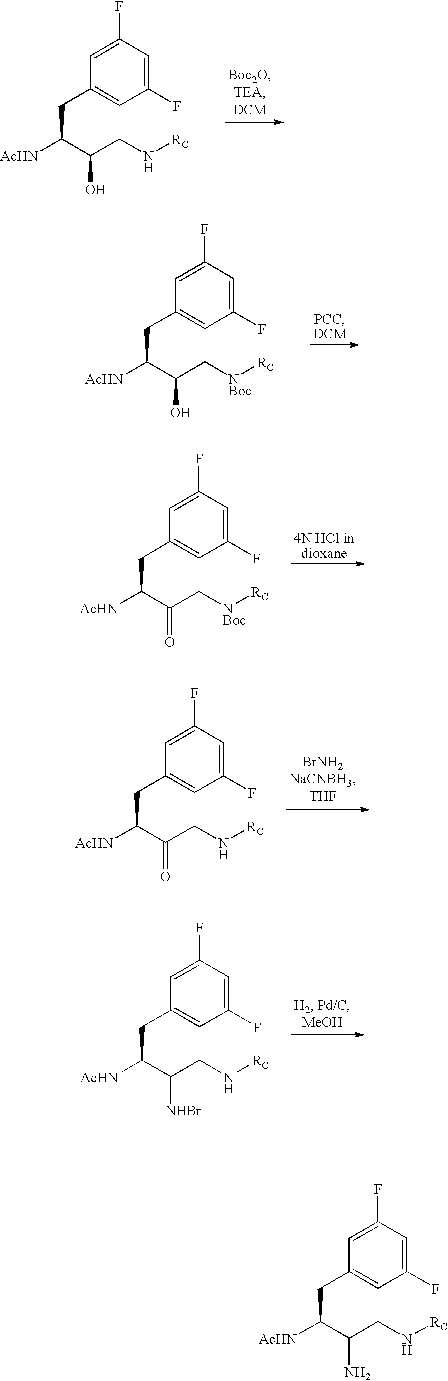

### Example 18

### ALTERNATIVE SCHEME FOR HYDROXYL REPLACEMENT

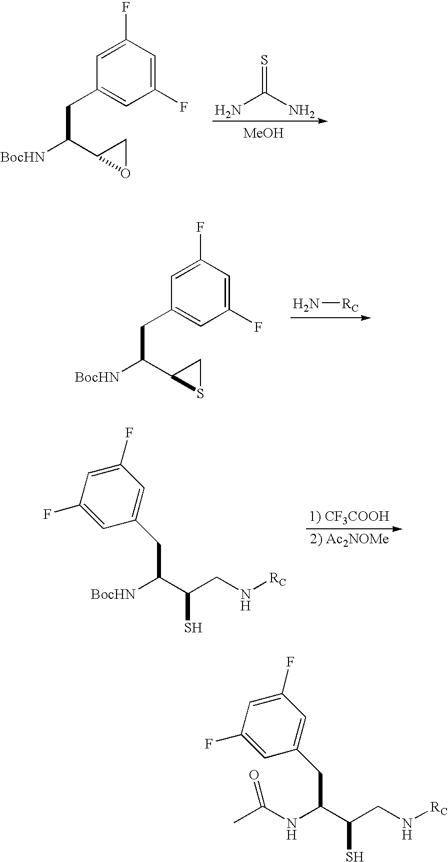

### Examples 19-34

### SYNTHESIS OF PRECURSORS

### Example 19

### PREPARATION OF PRECURSOR SUBSTITUTED AMINES

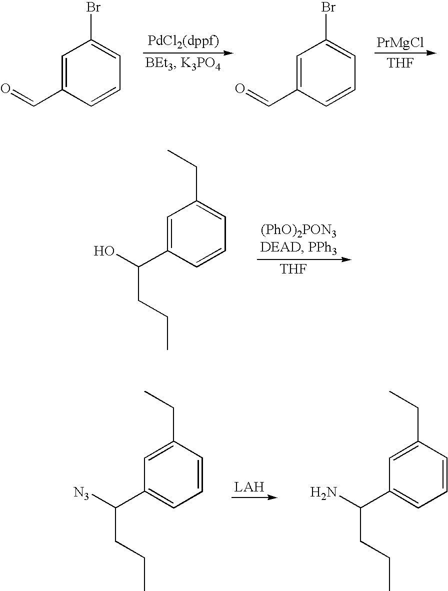

Precursor amines can generally be prepared as shown above. Specific examples are described below.

### Example 20

### PREPARATION OF TRIFLUORO-METHANESULFONIC ACID 2-AMINOMETHYL-4-(2,2-DIMETHYL-PROPYL)-PHENYL ESTER

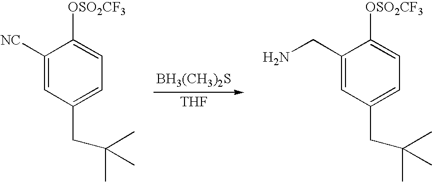

Trifluoro-methanesulfonic acid 2-cyano-4-(2,2-dimethyl-propyl)-phenyl ester (2.87 g, 8.93 mmol) in anhydrous THF (37 mL) was cooled to 0° C. under N₂(g) inlet. Borane-methyl sulfide complex (3.4 mL, 35.85 mmol) was added slowly to the reaction and refluxed overnight at 75° C. The reaction mixture was cooled to room temperature and quenched with 5N HCl. After stirring for 30 min the mixture was concentrated under reduced pressure. This was then partitioned between sat. NaHCO₃ (aq) and a solvent mixture of iPA/CHCl₃ (1:3). The organic layer was separated, dried (sodium sulfate) and concentrated under reduced pressure. The residue was purified by flash chromatography (Hexane:EtOAc, 1:1) yielding the desired amine (1.63 g, 56%): ¹H NMR (CDCl₃, 300 MHz) δ 7.26 (s, 1H), 7.15 (d, J=8.4 Hz, 1H), 7.07 (d, J=6.6 Hz, 1H), 3.94 (s, 2H), 2.51 (s, 2H), 1.54 (s, 2H), 0.91 (s, 9H).

### Examples 21-26

### SYNTHESIS OF PYRIDINE DERIVATIVES

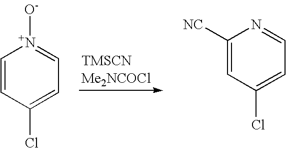

The nitrile was introduced essentially according to the method of Ornstein, P. L. et al. J. Med. Chem., 1991, 34, 90-97. The crude product was filtered through silica (CH₂Cl₂ elution) yielding the product as a white crystalline solid: ¹H NMR (300 MHz, CDCl₃) δ 8.64 (d, J=5.3 Hz, 1H), 7.72 (d, J=1.7 Hz, 1H), 7.56 (dd, J=5.3, 1.7 Hz, 1H); Cl MS m/z 139.0 [M+H]⁺(³⁵Cl).

### Example 21

### PREPARATION OF 2-CYANO-4-ISOPROPYLPYRIDINE

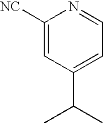

2-Cyano-4-isopropylpyridine was synthesized according to the method of Ornstein, P. L. et al. J. Med. Chem., 1991, 34, 90-97: Cl MS m/z 147.1 [M+H]⁺.

### Example 22

### PREPARATION OF 2-CYANO-4-TERT-BUTYLPYRIDINE

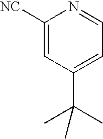

2-Cyano-4-tert-butylpyridine was synthesized according to the method of Ornstein, P. L. et al. J. Med. Chem., 1991, 34, 90-97: ¹H NMR (300 MHz, CDCl₃) δ 8.60 (d, J=5.3 Hz, 1H), 7.68 (d, J=1.5 Hz, 1H), 7.49 (dd, J=5.3,1.9 Hz, 1H), 1.33 (s, 9H); Cl MS m/z 161.1 [M+H]⁺.

### Example 23

### PREPARATION OF 2-CYANO-6-NEOPENTYLPYRIDINE

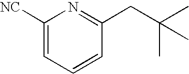

2-Cyano-6-neopentylpyridine was synthesized from 2-neopentylpyridine according to the method of Ornstein, P. L. et al. J. Med. Chem., 1991, 34, 90-97: R_{f}=0.62 in 20% ethyl acetate/hexanes; Cl MS m/z 175.1 [M+H]⁺.

### Example 24

### PREPARATION OF 2-NEOPENTYLPYRIDINE FROM 2-BROMOPYRIDINE

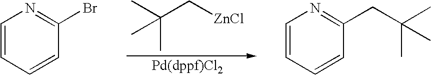

A solution of neopentylzinc chloride was prepared according to the method of Negishi, E.-I. et al. Tetrahedron Lett., 1983, 24, 3823-3824.

2-Bromopyridine (0.48 mL, 5.0 mmol) and [1,1′-bis(diphenylphosphino) ferrocene]dichloropalladium(II), complex with CH₂Cl₂ (1:1) (200 mg, 0.25 mmol) were added to the neopentylzinc chloride suspension. The resulting suspension was stirred at room temperature for 21 h, whereupon saturated ammonium chloride solution (25 mL) was added. The mixture was extracted with ethyl acetate. The combined organic extracts were dried (sodium sulfate), filtered and concentrated under reduced pressure. The residue was dissolved in CH₂Cl₂ and washed with 1 N HCl. The aqueous layer was separated, basified with 10 N NaOH (aq), and extracted with CH₂Cl₂. The organic layer was dried (sodium sulfate), filtered and concentrated under reduced pressure yielding 2-neopentylpyridine as an oil: R_{f}=0.33 in 5% MeOH/CH₂Cl₂.

### Example 25

### PREPARATION OF 2-CYANO-4-NEOPENTYLPYRIDINE

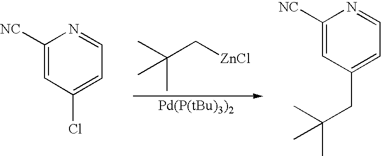

This transformation was performed according to the method of Dai, C. and Fu, G. J. Am. Chem. Soc., 2001, 123, 2719-2724. The crude residue was purified by filtration through a small plug of silica (20% ether/hexanes elution) yielding 2-cyano-4-neopentylpyridine: R_{f}=0.25 in 20% Et₂O/hexanes; Cl MS m/z 175.1 [M+H]⁺.

### Example 26

### PREPARATION OF 4-CYANO-2-NEOPENTYLPYRIDINE

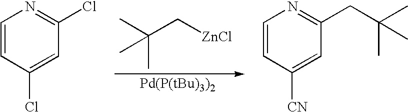

The method for the synthesis of 2-cyano-4-neopentylpyridine described in Example 25 was used to convert 2-chloro-4-cyanopyridine (Oakwood) into 4-cyano-2-neopentylpyridine: R_{f}=0.47 in 10% ethyl acetate/hexanes; ¹H NMR (300 MHz, CDCl₃) δ 8.73 (dd, J=4.9, 0.7 Hz, 1H), 7.55-7.40 (m, 2H), 2.75 (s, 2H), 0.96 (s, 9H); Cl MS m/z 175.1 [M+H]⁺.

### Example 27

### PREPARATION OF 5-BROMO-2-(1H-IMIDAZOL-1-YL)BENZONITRILE

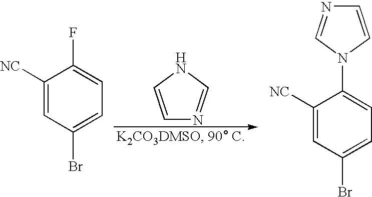

K₂CO₃ (3.337 g, 24.4 mmol) was added to a stirred solution of 5-bromo-2-fluorobenzonitrile (2.5 g, 12.2 mmol) in DMSO (50 mL), followed by the addition of 1H-imidazole (996 mg, 14.64 mmol). The reaction mixture was heated to 90° C. overnight, and diluted with water. The reaction mixture was extracted with ethyl acetate. The organic layer was washed with water and brine, dried with sodium sulfate, filtered, and concentrated under reduced pressure to yield the imidazolylbenzonitrile as an off-white solid (2.97 g, 98% yield). ¹H NMR (CDCl₃) δ 7.97 (m, 2H), 7.90 (m, 1H), 7.41 (d, J=8 Hz, 1H), 7.37 (s, 1H), 7.32 (s, 1H).

### Example 28

### PREPARATION OF 5-(2,2-DIMETHYLPROPYL)-2-IMIDAZOL-1-YL-BENZONITRILE

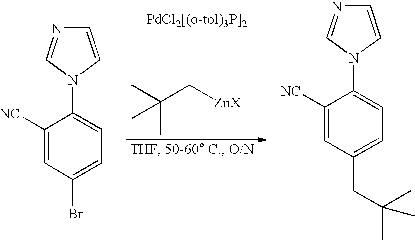

Neopentyl iodide (25.4 mL, 191 mmol) was added to a stirred Rieke Zn suspension (250 mL of a 5 g/100 mL THF solution, 191 mmol) at room temperature. The mixture was heated to 50° C. for 3 h, then dichlorobis(tri-o-tolylphosphine)palladium(II) (5.0 g, 6.4 mmol) and 5-bromo-2-(1H-imidazol-1-yl)benzonitrile (16 g, 64.5 mmol) were added in portions to the stirring suspension at 50° C. The reaction mixture was heated at 50-60° C. for 17 h.

The reaction was quenched with 1 N HCl, then filtered through celite, and separated. The organic layer was washed with water, and then 1 N HCl. The acidic extracts were combined, and basified with 10 N NaOH to pH 12. The resulting aqueous suspension was extracted(ethyl acetate). The combined extracts were washed with brine, dried (sodium sulfate), filtered, and concentrated under reduced pressure. The crude material (7 g) was taken to subsequent reaction without further purification: MS m/z 240.1 [M+H]⁺.

### Example 29

### PREPARATION OF 5-(2,2-DIMETHYLPROPYL)-2-IMIDAZOL-1-YL-BENZYLAMINE

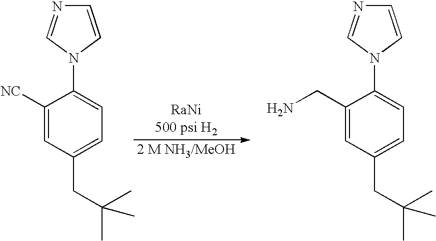

Raney nickel suspension (50% in water, ˜2 mL) was added to 5-(2,2-dimethylpropyl)-2-imidazol-1-yl-benzonitrile (5.3 g, 22.1 mmol) in 2 M ammonia in methanol (˜180 mL) in a pressure bomb. The vessel was sealed and pressurized with 500 psi H₂ for 16 h at room temperature. The reaction mixture was filtered through celite and concentrated under reduced pressure. The crude product was purified by flash chromatography (0-20% MeOH/CH₂Cl₂) yielding the product as a slightly red oil (2.5 g, 42%).

### Example 30

### PREPARATION OF FLUOROACETYL IMIDAZOLE

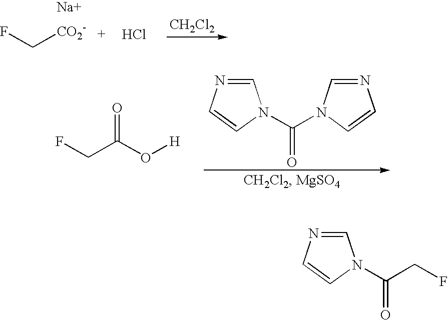

Concentrated HCl (1 mL, 12 mmol) was added to a slurry of sodium fluoroacetate (1.2 g, 12 mmol) in 25 mL of CH₂Cl₂, while the flask is swirled. The mixture was dried (magnesium sulfate), filtered, placed under N₂(g), and carbonyldiimidazole (1.3 g, 8 mmol) was added over 20 min. After 1 h, magnesium sulfate was added, and the mixture was allowed to stir overnight. It was then filtered and concentrated under reduced pressure yielding a pale yellow oil (1.6 g) containing CH₂Cl₂, fluoroacetic acid, imidazole, and fluoroacetyl imidazole: ¹H NMR (CDCl₃) δ 8.26 (s, 1H), 7.53 (s, 1H), 7.15 (s, 1H), 5.40 (d, J=47 Hz, 2H). Integration revealed the oil to be 28% by weight fluoroacetyl imidazole (0.45 g, 3.5 mmol, 44%). The oil was diluted with CH₂Cl₂ to make a solution that is 0.2 M fluoroacetyl imidazole.

### Example 31

### SYNTHESIS OF 5-CARBOETHOXY-2-ISO-BUTYLTHIAZOLE

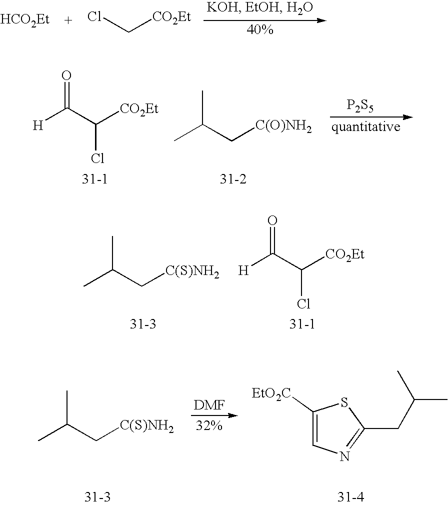

Claisen condensation of ethyl formate and ethyl chloroacetate gave ester 31-1. Treatment of isovaleramide 31-2 with phosphorus pentasulfide yielded 3-methyl-thiobutyramide 31-3. Cyclization of 31-1 and 31-3 yielded 5-carboethoxy-2-iso butylthiazole 31-4.

### STEP 1

A solution of ethyl formate (38 mL, 470 mmol) and ethyl chloroacetate (44 mL, 416 mmol) in diethyl ether (200 mL) was added to an ice-cold solution of potassium ethoxide (33.5 g, 400 mmol) in 1:2 ethyl alcohol/diethyl ether (300 mL). The suspension was stirred overnight at room temperature. The solid was filtered, washed with diethyl ether, and dissolved in water. The solution was cooled in an ice bath and acidified to pH 4 with concentrated HCl. The solution was extracted with diethyl ether, washed with saturated NaCl, dried (sodium sulfate), filtered, and concentrated under reduced pressure yielding formylchloroacetate 31-1 (24.2 g, 40%) as a yellow oil: ¹H NMR (300 MHz, CDCl₃) δ 4.99-4.19 (m, 2H), 4.08 (s, 1H), 3.64-3.57 (m, 1H), 1.35-1.18 (m, 3H).

STEP 2:

Phosphorus pentasulfide (3.8 g, 10.9 mmol) was added in portions to a solution of isovaleramide 31-2 (10 g, 99 mmol) in diethyl ether (400 mL). The reaction mixture was stirred at room temperature for 2 h and then filtered. The filtrate was concentrated under reduced pressure yielding isovalerothioamide 31-3 (11.60 g, quantitative) as a yellow oil: ¹H NMR (300 MHz, DMSO-d₆) δ 9.34 (s, 1H), 9.12 (s, 1H), 2.33 (d, J=7.3 Hz, 2H), 2.17-2.12 (m, 1H), 0.86 (d, J=8.4 Hz, 6H).

### STEP 3

A solution of 31-3 (11.60 g, 98.97 mmol) and 31-1 (9.98 g, 66.31 mmol) in N,N-dimethylformamide (40 mL) was heated at 95° C. overnight. The reaction mixture was cooled to 0° C. and cold water (100 mL) added. The reaction mixture was adjusted to pH 8 by slow addition of solid sodium bicarbonate and extracted with diethyl ether, washed with water, saturated NaCl, dried (sodium sulfate), filtered, and concentrated under reduced pressure. Purification by flash column chromatography (90:10 hexanes/ethyl acetate) gave 5-carboethoxy-2-iso-butylthiazole 31-4 (4.53 g, 32%) as a yellow oil: ¹H NMR (300 MHz, CDCl₃) δ 4.36 (q, J=7.2 Hz, 2H), 2.90 (d, J=7.2 Hz, 2H), 2.15 (m, 1H), 1.38 (t, J=7.2 Hz, 3H), 1.06 (d, J=6.7 Hz, 6H).

### Example 32

### ALTERNATIVE PREPARATION OF 5-(2,2-DIMETHYL-PROPYL)-2-IMIDAZOL-1-YL-BENZYLAMINE

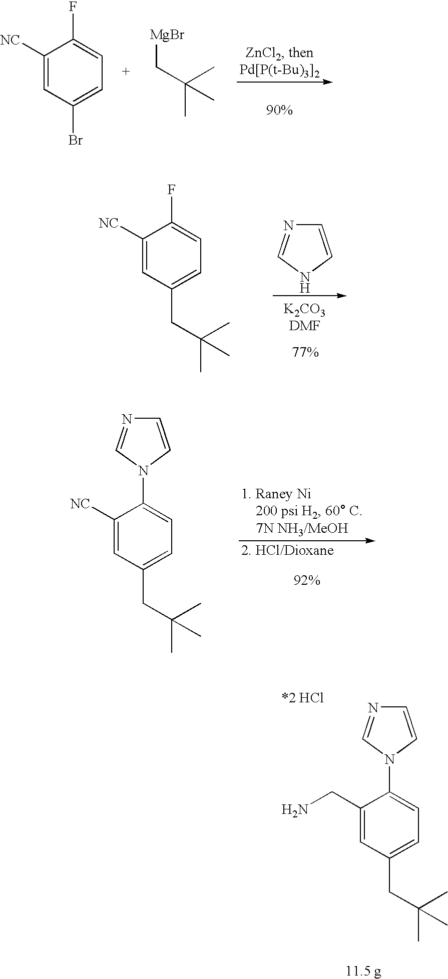

Incorporation of the neopentyl group was performed using a Negishi coupling with the neopentyl zinc species generated from the commercially available neopentylmagnesium chloride. The in situ generated neopentyl zinc reagent underwent cross-coupling reaction with the aryl bromide using the Fu catalyst at room temperature. Displacement of the aryl fluoride with imidazole occurred in DMF with heating. Reduction of the nitrile-was carried out with Raney Ni. During the reduction, a significant amount of dimer was seen when Boc anhydride was used instead of ammonia. The reaction was found to proceed to completion at 200 psi of hydrogen at 60° C. Reduction of the temperature to either 20° C. or 40° C. or reducing the pressure of H₂(g) significantly reduced the rate of the reduction. The product was an oil, but treating with HCl in dioxane gave the salt as a free flowing solid.

### STEP 1: Preparation of 5-neopentyl-2-fluoro-benzonitrile.

To a solution of zinc chloride (50 mL, 1.0M in diethyl ether, 50 mmol) was added neopentylmagnesium chloride (50 mL, 1.0M in THF, 50 mmol) dropwise at 0° C. During the addition, the generated magnesium salts formed a white precipitate. The reaction was removed from the ice bath and allowed to stir for 1 h then 1-bromo-2-fluorobenzonitrile (5 g, 25 mmol) was added followed by bis(tri-tert-butylphosphine) palladium (0.127 g, 0.25 mmol, 1%). The reaction began to reflux and was placed back into the ice bath. After 1 h, the reaction was diluted with 200 mL of diethyl ether and washed with 1N HCl (2×100 mL), brine (100 mL), dried over magnesium sulfate and concentrated to give an oily solid (4.3 g, 22 mmol, 90%).

### STEP 2: Preparation of 5-neopentyl-2-imidazol-1-yl-benzonitrile

A solution of 5-neopentyl-2-fluoro-benzonitrile (4.3 g, 22.5 mmol), imidazole (1.68 g, 24.73 mmol) and potassium carbonate (6.25 g, 44.97 mmol) were stirred in DMF (50 mL) at 90° C. The reaction was stopped after 4 h and worked up, but LCMS and ¹H NMR show starting material remaining. The crude product was resubmitted to reaction conditions and stirred overnight. The reaction was diluted with ethyl acetate (100 mL) and washed with water (2×75 mL) and brine (75 mL). The organic layer was dried over magnesium sulfate and concentrated to give a white solid (4.16 g, 17.4 mmol, 77%); MH+240.2.

### STEP 3: Preparation of 5-neopentyl-2-fluoro-benzylamine.

To a solution of 5-neopentyl-2-imidazol-1-yl-benzonitrile (10.00 g, 41.79 mmol) in ammonia in methanol solution (7 N, 350 mL) was added a slurry of Raney nickel (10 mL). The reaction was sealed in a parr bomb and placed under H₂ (200 psi) then heated to 60° C. As the pressure dropped, H₂ was added to adjust the pressure to 200 psi. After 8 h, the vessel was cooled, the hydrogen was removed and the reaction was placed under N₂(g). The reaction was filtered, washed with methanol and concentrated. The resulting oil was dried for 48 h. The oil was dissolved in 50 mL of diethyl ether and 4N HCl in dioxane (32 mL) was added which caused a precipitate to form. This precipitate was collected by filtration, washed with diethyl ether (100 mL) and CH₂Cl₂ (100 mL). Drying under high vacuum gave a white solid (12.1 g, 38.3 mmol, 92%); MH+244.2.

### Example 33

### ALTERNATIVE PREPARATION OF [2-(3,5-DIFLUORO-PHENYL)-1-OXIRANYL-ETHYL]-CARBAMIC ACID TERT-BUTYL ESTER

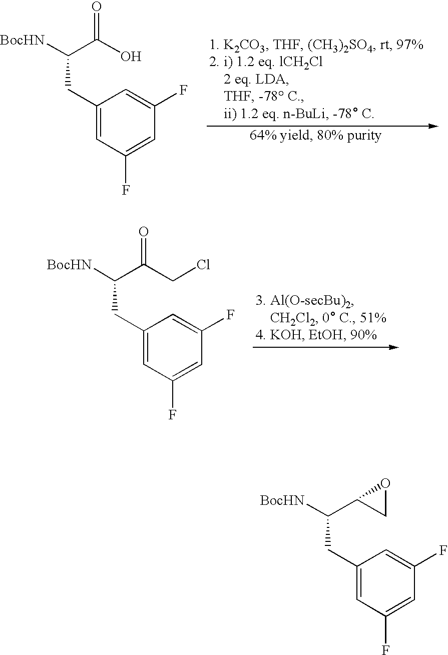

The synthesis of tert-butyl (1S)-2-(3,5-difluorophenyl)-1-[(2S)-oxiranyl]ethylcarbamate was carried out using the procedure described by Reeder. (2S)-2-[(tert-butoxycarbonyl)amino]-3-(3,5-difluorophenyl)propionic acid was purchased from Chem Impex and converted to the methyl ester without incident. Conversion of the methyl ester to the chloroketone was carried out on a 50 g scale and repeatedly gave yields between 60-65% of an impure product. The chlorohydrin was obtained via a diastereoselective Meerwein-Ponndorf-Verley reduction. The product was washed with octane to remove some, but not all, of the impurities. Conversion of the chlorohydrin to the epoxide occurred with potassium hydroxide in ethanol with the product being isolated from the reaction mixture by precipitation after the addition of water. The epoxide could be recrystallized from hexanes/isopropanol, although some batches of epoxide contained an unidentified impurity.

### STEP 1: Preparation of (2S)-2-[(tert-Butoxycarbonyl)amino]-3-(3,5-difluorophenyl)propionic acid methyl ester

A solution of (2S)-2-[(tert-butoxycarbonyl)amino]-3-(3,5-difluorophenyl)propionic acid (138 g, 458 mmol) was dissolved in THF (1000 mL) and cooled to 0° C. Potassium carbonate (69.6 g, 503.8 mmol) was added followed by the dropwise addition of dimethyl sulfate (45.5 mL, 480.9 mmol). The reaction was removed from the ice bath and allowed to stir at room temperature overnight after which HPLC analysis shows the complete consumption of starting material. The reaction was quenched by the addition of 10% ammonium hydroxide (150 mL). The aqueous layer was removed and extracted with ethyl acetate (500 mL). The combined organics were washed with brine (500 mL), dried over magnesium sulfate and concentrated to give a yellow solid. The solid was recrystallized from hexanes to give the product as an off white solid (140.3 g, 445.0 mmol, 97%).

### STEP 2: tert-Butyl (1S)-3-chloro-1-(3,5-difluorobenzyl)-2-oxopropylcarbamate

A solution of LDA was prepared by adding n-BuLi (26 mL, 260 mmol) to a solution of diisopropylamine (26.3 g, 260 mmol) in THF (200 mL) at −78° C. After the addition was complete, the reaction was allowed by warm to 0° C. This light yellow solution was added dropwise to a solution of (2S)-2-[(tert-butoxycarbonyl)amino]-3-(3,5-difluorophenyl)propionic acid methyl ester (40 g, 127 mmol) and chloroiodomethane (11.1 mL, 152 mmol) keeping the temperature below −65° C. After the addition, the solution was stirred for 30 min at −78° C. n-BuLi (15 mL, 150 mmol) was added dropwise keeping the internal temperature below −62° C. The reaction was stirred for 30 min at −78° C. then quenched into 500 mL of 1N HCl at 0° C. The product was extracted into EtOAc (500 mL), washed with brine (300 mL), dried over magnesium sulfate and concentrated. Octane (400 mL) was added to the product and the resulting solid collected by filtration and dried. The octane was cooled to −78° C. then allowed to warm until the octane melted. The resulting solid was collected and added to the previously collected solid. Drying of the combined solid gave the title compound as an off-white solid (33.9 g, 101.5 mmol, 64.5%).

### STEP 3: tert-Butyl (1S, 2S)-3-chloro-1-(3,5-diflurorbenzyl)-2-hydroxypropylcarbamate

A solution of tert-butyl (1S)-3-chloro-1-(3,5-difluorobenzyl)-2-oxopropylcarbamate (67.4 g, 202 mmol) was dissolved in DCM (500 mL) and cooled to 0° C. Tri(sec-butoxy)aluminum (54.7 g, 222.1 mmol, 1.1 eq) in DCM (50 mL) was added dropwise. After stirring for 2 h at 0° C., the reaction was complete by HPLC. The reaction was quenched with 1N HCl (750 mL) and the product extracted into ethyl acetate (2×400 mL). The combined organics were washed with brine (500 mL), dried over magnesium sulfate and concentrated to give an oily yellow solid. Octane (300 mL) was added and the resulting solid was collected by filtration and washed with octane (100 mL). Drying overnight gave a white solid. The octane layers were collected and concentrated to about 100 mL of volume, then placed in the freezer for 48 h to yield a second crop of the title compound (35 g, 104 mmol, 51%).

### STEP 4: tert-Butyl (1S)-2-(3,5-diflurorphenyl)-1-[(2S)-oxiranyl]ethylcarbamate

A solution of tert-butyl (1S, 2S)-3-chloro-1-(3,5-diflurorbenzyl)-2-hydroxypropylcarbamate in ethanol (150 mL) was cooled to 0° C. A solution of KOH in EtOH (25 mL) was added. The reaction was removed from the ice bath and stirred for 2 h. The reaction was diluted with 300 mL of water and placed into an ice bath. The resulting solid was collected by filtration and washed with cold water (100 mL). Drying overnight gave an off-white solid (6.74 g, 22.51 mmol, 90%).

### Example 34

### ALTERNATIVE PREPARATION OF 5-BROMO-2-IMIDAZOLE-1-YL-BENZONITRILE

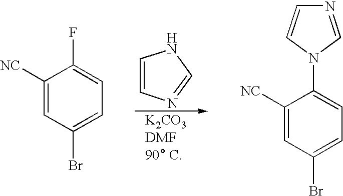

Several different solvents were used to filter off impurities from the crude product. Hexanes did not require additional purification.

To a stirred solution 5-bromo-2-fluorobenzonitrile (50.0 g, 250 mmol) in DMF (300 mL) was added K₂CO₃ (69 g, 500 mmol), and then imidazole (20.0 g, 300 mmol). The reaction mixture was heated to 90° C. and stirred overnight. The reaction mixture was diluted with water and extracted with EtOAc (2×). The organic layer was washed with water (1×) and brine (1×), dried with sodium sulfate, filtered, and concentrated. Hexane was added to the resulting solid and allowed to stir for 5 min then filtered off leaving a white solid.

### Example 35

### PREPARATION OF 1-[4-(2,2-DIMETHYL-PROPYL)-2-METHYL-PHENYL]-PYRROLIDIN-3-OL; 4-(3,5-DIFLUORO-PHENYL)-3-METHYL-1-METHYLAMINO-BUTAN-2-OL

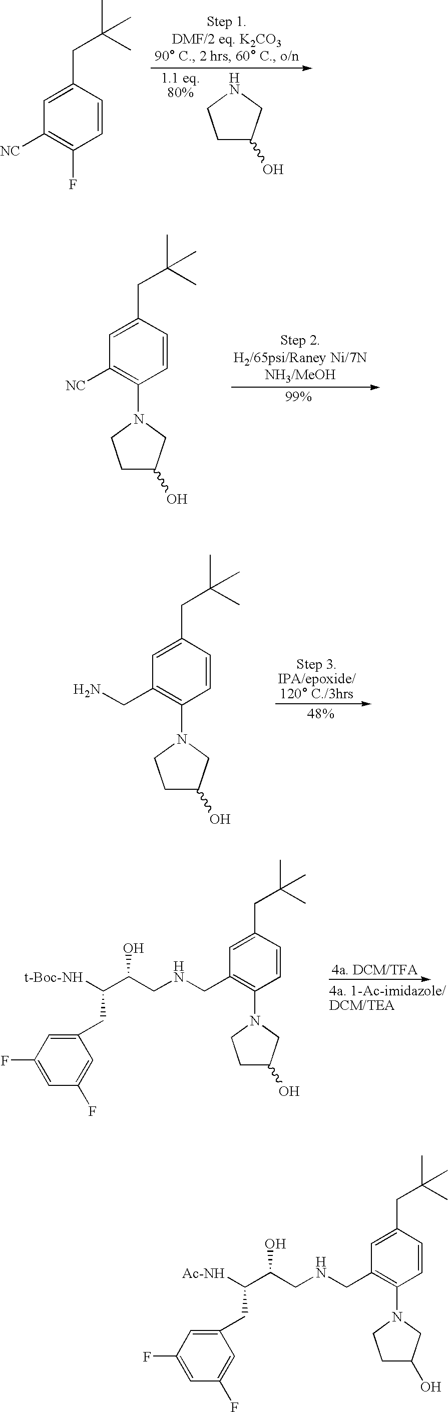

### Step 1. Preparation of 5-(2,2-Dimethyl-propyl)-2-(3-hydroxy-pyrrolidin-1-yl)-benzonitrile

To 0.76 g (4 mmol) of 2-fluoro-5-neopentylbenzonitrile in 15 mL of DMF was added 1.11 g (8 mmol, 2 eq.) of potassium carbonate and 0.43 mL (5.2 mmol, 1.3 eq.) of 3-pyrrolidinol and heated to 90-100° C. overnight. The reaction was monitored by HPLC/MS, Rt=1.349 min, m/e=259.2/281.2. The reaction was allowed to cool to room temperature and quenched with ice/water/DCM. It was then extracted and washed with brine, dried, stripped of solvent and purified by flash column to give 0.82 g of 5-(2,2-Dimethyl-propyl)-2-(3-hydroxy-pyrrolidin-1-yl)-benzonitrile (80% yield).

TLC (30% EtOAc/Hexane). Rf=0.16 where s. m. at Rf=0.84. LCMS m/e=259.2(M+H), Rt (retention time, minutes)=1.349. Analytical method: 50% [B]: 50% [A] to 95% [B]: 5% [A] gradient in 2.5 min, then hold, at 2 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.

### Step 2. Preparation of 1-[2-Aminomethyl-4-(2,2-dimethyl-propyl)-phenyl]-pyrrolidin-3-ol

To 0.8 g (3.1 mmol) of 5-(2,2-Dimethyl-propyl)-2-(3-hydroxy-pyrrolidin-1-yl)-benzonitrile in 27 mL of 7 M NH₃/methanol was added 1 g of Raney 2800 Ni/water in a Parr bottle, saturated with hydrogen to 65 psi and shaken overnight. The reaction mixture was filtered through a cake of celite and the solvents/ammonia were stripped off to give 0.82 g of 1-[2-Aminomethyl-4-(2,2-dimethyl-propyl)-phenyl]-pyrrolidin-3-ol. (99% yield).

LCMS m/e=246.2(M−NH₂), Rt (retention time, minutes)=1.324. Analytical Method: 20% [B]: 80% [A] to 70% [B]: 30% [A] gradient in 1.75 min, then hold, at 2 mL/min, where [A]=0.1% trifluoroacetic acid in water; [B]=0.1% trifluoroacetic acid in acetonitrile on a Phenomenex Luna C18 (2) 4.6 mm×30 cm column, 3 micron packing, 210 nm detection, at 35° C.).

### Step 3. Preparation of 1-[4-(2,2-Dimethyl-propyl)-2-methyl-phenyl]-pyrrolidin-3-ol; 4-(3,5-difluoro-phenyl)-3-methyl-1-methylamino-butan-2-ol

LCMS m/e=504.3(M+H), Rt (retention time, minutes)=2.168 (Using the method from step 2.).

### Step 4. 1-[4-(2,2-Dimethyl-propyl)-2-methyl-phenyl]-pyrrol idin-3-ol; 4-(3,5-difluoro-phenyl)-3-methyl-1-methylamino-butan-2-ol

LCMS m/e=457.2(M+H), Rt (retention time, minutes)=1.761 (Using the method from step 2.).

¹H NMR (CDCl₃) δ 7.33-7.30 (d, J=8.8 Hz, 1H), 7.27-7.15 (m, 2H), 6.97-6.57 (m, 3H), 4.42-4.38 (d, J=14.2 Hz, 1H), 4.19-4.14 (d, J=14.3 Hz, 1H), 4.01-3.95 (m, 2H), 3.42-2.86 (m, 8H), 2.68-2.51 (m, 3H), 2.45 (s, 2H), 2.33-2.31 (m, 1H), 1.99-1.95 (m, 1H), 1.83 (s, 3H), 0.87 (s, 9H). ¹³C NMR (CDCl₃) δ 172.4, 164.7, 161.2, 144.7, 141.8, 137.9, 133.3, 132.9, 124.1, 120.3, 112.1, 111.79, 102.06, 77.4, 77.0, 76.6, 70.4, 69.1, 60.8, 53.2, 50.9, 50.4, 50.1, 49.2, 35.3, 33.4, 31.6, 29.1, 22.4.

### Example 36

### PREPARATION OF N-{1-(3,5-DIFLUORO-BENZYL)-3-[5-(2,2-DIMETHYL-PROPYL)-2-PYRROLIDIN-1-YL-BENZYLAMINO]-2-HYDROXY-PROPYL}-ACETAMIDE

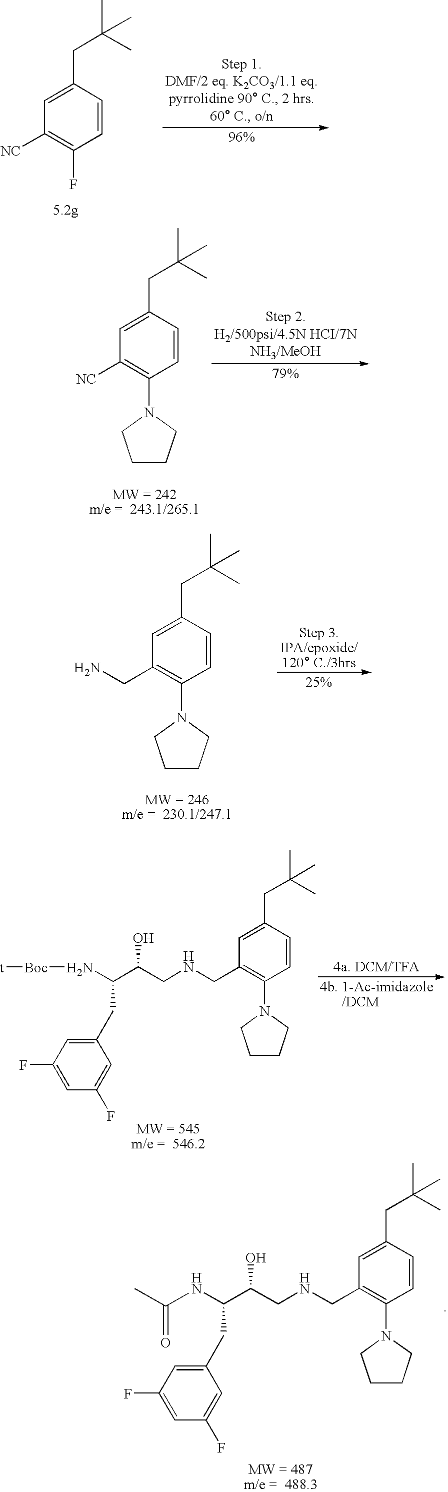

N-{1-(3,5-difluoro-benzyl)-3-[5-(2,2-dimethyl-propyl)-2-pyrrolidin-yl-benzylamino]-2-hydroxy-propyl}-acetamide was prepared essentially according to the protocol in Example 35.

### Step 1. Preparation of 5-(2,2-Dimethyl-Propyl)-2-Pyrrolidin-1-Yl-Benzonitrile

LCMS m/e=243.1/265.1 (M+H), Rt (retention time, minutes)=2.436. (Using the method from Example 35, step 2.).

### Step 2. Preparation of 5-(2,2-Dimethyl-propyl)-2-pyrrolidin-1-yl-benzylamine

LCMS m/e=230.1/247.1 (M+H), Rt (retention time, minutes)=1.528. (Using the method from Example 35, step 2.).

### Step 3. Prepartion of {1-(3,5-Difluoro-benzyl)-3-[5-(2,2-dimethyl-propyl)-2-pyrrolidin-1-yl-benzylamino]-2-hydroxy-propyl}-carbamic acid tert-butyl ester

LCMS m/e=546.2 (M+H), Rt (retention time, minutes)=2.463. (Using the method from Example 35, step 2.).

### Step 4. Prepartion of N-{1-(3,5-Difluoro-benzyl)-3-[5-(2,2-dimethyl-propyl)-2-pyrrolidin-1-yl-benzylamino]-2-hydroxy-propyl}-acetamide

LCMS m/e=488.3 (M+H), Rt (retention time, minutes)=1.904. (Using the method from Example 35, step 2.).

### Example 37

### PREPARATION OF 4-(3,5-DIFLUORO-BENZYL)-6-[5-(2,2-DIMETHYL-PROPYL)-2-PIPERIDIN-1-YL-BENZYLAMINO]-5-HYDROXY-HEXAN-2-ONE

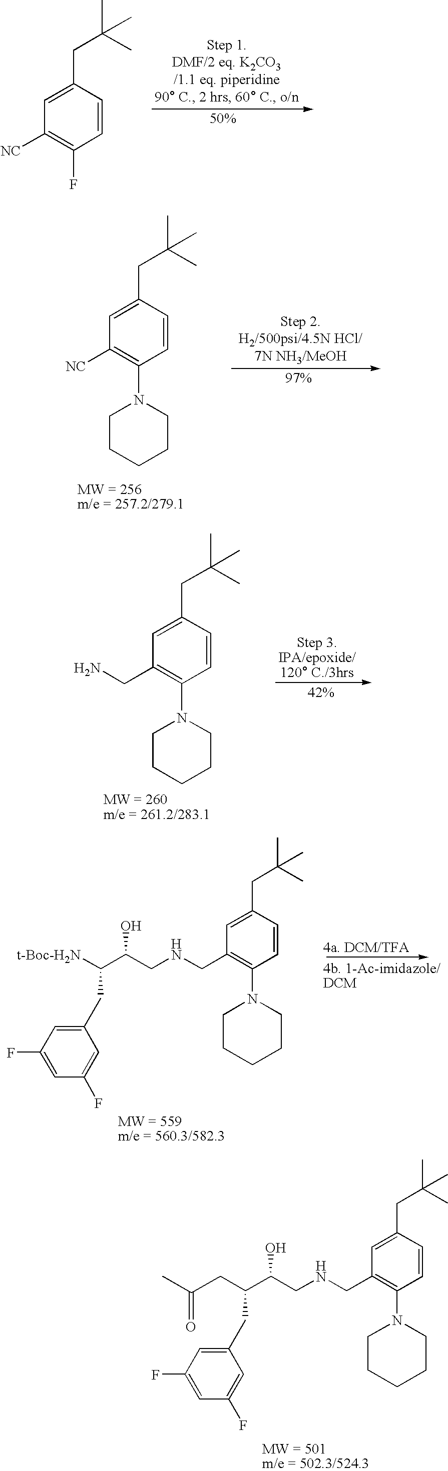

4-(3,5-Difluoro-benzyl)-6-[5-(2,2-dimethyl-propyl)-2-piperidin-1-yl-benzylamino]-5-hydroxy-hexan-2-one was prepared essentially according to the procedures in Examples 35 and 36.

### Step 1. Preparation of 5-(2,2-Dimethyl-propyl)-2-piperidin-1-yl-benzonitrile

LCMS m/e=257.1/279.1 (M+H), Rt (retention time, minutes)=2.599. (Using the method from Example 35, step 2.).

### Step 2. Preparation of 5-(2,2-Dimethyl-propyl)-2-piperidin-1-yl-benzylamine

LCMS m/e=261.2/283.1 (M+H), Rt (retention time, minutes)=1.358. (Using the method from Example 35, step 2.).

### Step 3. Preparation of {1-(3,5-Difluoro-benzyl)-3-[5-(2,2-dimethyl-propyl)-2-piperidin-1-yl-benzylamino]-2-hydroxy-propyl}-carbamic acid tert-butyl ester

LCMS m/e=560.3/582.3 (M+H), Rt (retention time, minutes)=2.422. Using the method from Example 35, step 2.).

### Step 4. Preparation of N-{1-(3,5-Difluoro-benzyl)-3-[5-(2,2-dimethyl-propyl)-2-piperidin-1-yl-benzylamino]-2-hydroxy-propyl}-acetamide

LCMS m/e=502.3/524.3 (M+H), Rt (retention time, minutes)=2.108. (Using the method from Example 35, step 2.).

### Example 38

### COPPER(I) CATALYZED N-ARYLATIONS: PREPARATION OF N-(1-(3,5-DIFLUOROPHENYL)-3-HYDROXY-4-(5-NEOPENTYL-2-(1H-PYRAZOL-1-YL)BENZYLAMINO)BUTAN-2-YL)ACETAMIDE

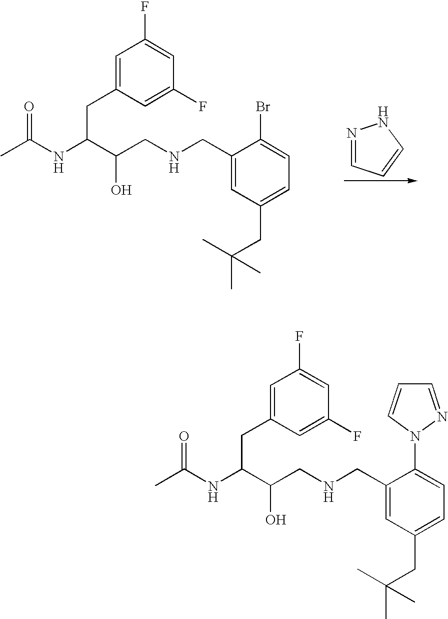

CuI (1.9 mg, 0.01 mmol) was added to a 4 mL vial containing pyrazole (8.2 mg, 0.12 mmol), Cs₂CO₃ (28 mg, 0.2 mmol), N-(4-(2-bromo-5-neopentylbenzylamino)-1-(3,5-difluorophenyl)-3-hydroxybutan-2-yl)acetamide (49.5 mg, 0.1 mmol), and dioxane (0.5 mL). The reaction was capped and shaken at 80° C. until the starting bromide was consumed (1 to 3 days as determined by LCMS). The dioxane was evaporated under N₂(g), acidified with 1N HCl in ethanol (100 μL), diluted (400 μL ethanol), and filtered. N-(1-(3,5-difluorophenyl)-3-hydroxy-4-(5-neopentyl-2-(1H-pyrazol-1-yl)benzylamino)butan-2-yl)acetamide was purified via RP-HPLC.

Analytical Protocol: 50 mm(long)×3 mm(i.d.), C-18 stationary phase, 5 micron particle size, 100 angstrom pore size. Mobile phases are 0.05% trifluoroacetic acid in water (solvent A), and 0.05% trifluoroacetic acid in acetonitrile. Chromatographic conditions are 3 mL/min: 5% solvent B from 0 to 0.275 min, 5% to 95% solvent B from 0.275 to 2.75 min, then 95% solvent B from 2.75 to 3.50 min.

### Example 39

### EXAMPLES OF COMPOUNDS SYNTHESIZED USING ANALOGOUS METHODS

The example compounds listed below can be synthesized using methods analogous to those described in Examples 5-34:

N-{1-(3,5-difluoro-benzyl)-3-[5-(2,2-dimethyl-propyl)-2-(2-propyl-imidazol-1-yl)-benzylamino]-2-hydroxy-propyl}-acetamide, N-(1-(3,5-difluorophenyl)-3-hydroxy-4-(5-neopentyl-2-(1H-pyrrol-2-yl)benzylamino)butan-2-yl)acetamide, N-(4-(2-(1H-imidazol-1-yl)-5-neopentylbenzylamino)-1-(3,5-difluorophenyl)-3-hydroxybutan-2-yl)acetamide, N-(1-(3,5-difluorophenyl)-3-hydroxy-4-(5-neopentyl-2-(1H-pyrazol-4-yl)benzylamino)butan-2-yl)acetamide, N-{1-(3,5-difluoro-benzyl)-3-[5-(2,2-dimethyl-propyl)-2-[1,2,3]thiadiazol-4-yl-benzylamino]-2-hydroxy-propyl}-acetamide, N-{1-(3,5-difluoro-benzyl)-3-[5-(2,2-dimethyl-propyl)-2-thiazol-5-yl-benzylamino]-2-hydroxy-propyl}-acetamide, N-{1-(3,5-difluoro-benzyl)-3-[5-(2,2-dimethyl-propyl)-2-(3-methyl-isothiazol-5-yl)-benzylamino]-2-hydroxy-propyl}-acetamide, N-{1-(3,5-difluoro-benzyl)-3-[5-(2,2-dimethyl-propyl)-2-(2H-[1 ,2,3]triazol-4-yl)-benzylamino]-2-hydroxy-propyl}-acetamide, N-(1-(3,5-difluorophenyl)-3-hydroxy-4-(5-neopentyl-2-(pyridin-3-yl)benzylamino)butan-2-yl)acetamide, N-(1-(3,5-difluorophenyl)-4-(2-(6-fluoropyridin-3-yl)-5-neopentylbenzylamino-3-hydroxybutan-2-yl)acetamide, N-{1-(3,5-difluoro-benzyl)-3-[5-(2,2-dimethyl-propyl)-2-(2-fluoro-pyridin-3-yl)-benzylamino]-2-hydroxy-propyl}-acetamide, N-{1-(3,5-difluoro-benzyl)-3-[5-(2,2-dimethyl-propyl)-2-pyridazin-3-yl-benzylamino]-2-hydroxy-propyl}-acetamide, N-{1-(3,5-difluoro-benzyl)-3-[5-(2,2-dimethyl-propyl)-2-pyrimidin-5-yl-benzylamino]-2-hydroxy-propyl}-acetamide, N-(1-(3,5-difluoro-benzyl)-3-{1-[5-(2,2-dimethyl-propyl)-2-imidazol-1-yl-phenyl]-cyclopropylamino}-2-hydroxy-propyl)-acetamide, N-{1-(3,5-difluoro-benzyl)-3-[5-(2,2-dimethyl-propyl)-2-pyrazin-2-yl-benzylamino]-2-hydroxy-propyl}-acetamide, N-{1(3,5-difluoro-benzyl)-3-[5-(2,2-dimethyl-propyl)-2-(5-ethyl-imidazol-1-yl)-benzylamino]-2-hydroxy-propyl}-acetamide, N-[3-[3-chloro-5-(2,2-dimethyl-propyl)-2-imidazol-1-yl-benzylamino]-1-(3,5-difluoro-benzyl)-2-hydroxy-propyl}-acetamide, N-{1-(3,5-difluoro-benzyl)-3-[5-(2,2-dimethyl-propyl)-2-tetrazol-1-yl-benzylamino]-2-hydroxy-propyl}-acetamide, N-{1-(3,5-difluoro-benzyl)-3-[5-(2,2-dimethyl-propyl)-2-imidazol-1-yl-benzylamino]-2-hydroxy-propyl}-acetamide, N-((1S,2R)-1-(3,5-difluorobenzyl)-2-hydroxy-3-{[(4-isobutyl-1,1′-biphenyl-2-yl)methyl]amino}propyl) acetamide, N-{(1S,2R)-1-(3,5-difluorobenzyl)-2-hydroxy-3-[(5-isobutyl-2-pyridin-3-ylbenzyl)amino]propyl} acetamide, N-{(1S,2 R)-1-(3,5-difluorobenzyl)-2-hydroxy-3-[(5-isobutyl-2-pyridin-4-ylbenzyl)amino]propyl}acetamide, N-((1S,2R)-1-(3,5-difluorobenzyl)-3-{[(4′-fluoro-4-isobutyl-1,1′-biphenyl-2-yl)methyl]amino}-2-hydroxypropyl)acetamide, N-((1S,2R)-1-(3,5-difluorobenzyl)-3-{[(2′-fluoro-4-isobutyl-1,1′-biphenyl-2-yl)methyl]amino)-2-hydroxypropyl)acetamide, N-((1S,2R)-1-(3,5-difluorobenzyl)-2-hydroxy-3-{[5-isobutyl-2-(6-methoxypyridin-3-yl)benzyl]amino}propyl) acetamide, N-((1S,2R)-1-(3,5-difluorobenzyl)-2-hydroxy-3-{[(3′-hydroxy-4-isobutyl-1,1′-biphenyl-2-yl)methyl]amino} propyl)acetamide, N-[(1 S ,2R)-3-{[(3′-acetyl-4-isobutyl-1,1′-biphenyl-2-yl)methyl]amino}-1-(3,5-difluorobenzyl)-2-hydroxypropyl]acetamide, N-((1S,2R)-1-(3,5-difluorobenzyl)-2-hydroxy-3-{[5-isobutyl-2-(5-methoxypyridin-3-yl)benzyl]amino}propyl) acetamide, N-((1S,2R)-1-(3,5-difluorobenzyl)-3-{[2-(3-furyl)-5-isobutylbenzyl]amino}-2-hydroxypropyl) acetamide, and N-((1S,2R)-1-(3,5-difluorobenzyl)-3-{[2-(2-furyl)-5-isobutylbenzyl]amino}-2-hydroxypropyl)acetamide.

Generally, the protection of amines is conducted, where appropriate, by methods known to those skilled in the art. See, for example, Protecting Groups in Organic Synthesis, John Wiley and sons, New York, N.Y., 1981, Chapter 7; Protecting Groups in Organic Chemistry, Plenum Press, New York, N.Y., 1973, Chapter 2. When the amino protecting group is no longer needed, it is removed by methods known to those skilled in the art. By definition the amino protecting group must be readily removable. A variety of suitable methodologies are known to those skilled in the art; see also T. W. Green and P. G. M. Wuts, Protective Groups in Organic Chemistry, John Wiley and Sons, 3^{rd} edition, 1999. Suitable amino protecting groups include t-butoxycarbonyl, benzyl-oxycarbonyl, formyl, trityl, phthalimido, trichloro-acetyl, chloroacetyl, bromoacetyl, iodoacetyl, 4-phenylbenzyloxycarbonyl, 2-methylbenzyloxycarbonyl, 4-ethoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, 2-(4-xenyl) isopropoxycarbonyl, 1,1-diphenyleth-1-yloxycarbonyl, 1,1-diphenylprop-1-yloxycarbonyl, 2-phenylprop-2-yloxycarbonyl, 2-(p-toluyl)prop-2-yloxy-carbonyl, cyclopentanyloxycarbonyl, 1-methylcyclo-pentanyloxycarbonyl, cyclohexanyloxycarbonyl, 1-methyl-cyclohexanyloxycabonyl, 2-methylcyclohexanyloxycarbonyl, 2-(4-toluylsulfonyl)ethoxycarbonyl, 2-(methylsulfonyl)-ethoxycarbonyl, 2-(triphenylphosphino)ethoxycarbonyl, fluorenylmethoxycarbonyl, 2-(trimethylsilyl)ethoxy-carbonyl, allyloxycarbonyl, 1-(trimethylsilylmethyl)prop-1-enyloxycarbonyl, 5-benzisoxalylmethoxycarbonyl, 4-acetoxybenzyloxycarbonyl, 2,2,2-trichloroethoxycarbonyl, 2-ethynyl-2-propoxycarbonyl, cyclopropylmethoxycarbonyl, 4-(decyloxyl)benzyloxycarbonyl, isobornyloxycarbonyl, 1-piperidyloxycarbonyl, 9-fluoroenylmethyl carbonate, —CH—CH═CH₂, and the like.

In an embodiment, the protecting group is t-butoxycarbonyl (Boc) and/or benzyloxycarbonyl (CBZ). In another embodiment, the protecting group is Boc. One skilled in the art will recognize suitable methods of introducing a Boc or CBZ protecting group and may additionally consult Protective Groups in Organic Chemistry, for guidance.

The compounds of the present invention may contain geometric or optical isomers as tautomers. Thus, the present invention includes all tautomers and pure geometric isomers, such as the E and Z geometric isomers, as mixtures thereof. Further, the present invention includes pure enantiomers, diastereomers and/or mixtures thereof, including racemic mixtures. The individual geometric isomers, enantiomers or diastereomers may be prepared or isolated by methods known to those in the art, including, for example chiral chromatography; preparing diastereomers, separating the diastereomers and then converting the diastereomers into enantiomers.

Compounds of the present invention with designated stereochemistry can be included in mixtures, including racemic mixtures, with -other enantiomers, diastereomers, geometric isomers or tautomers. In a preferred embodiment, compounds of the present invention are typically present in these mixtures in diastereomeric and/or enantiomeric excess of at least 50%. Preferably, compounds of the present invention are present in these mixtures in diastereomeric and/or enantiomeric excess of at least 80%. More preferably, compounds of the present invention with the desired stereochemistry are present in diastereomeric and/or enantiomeric excess of at least 90%. Even more preferably, compounds of the present invention with the desired stereochemistry are present in diastereomeric and/or enantiomeric excess of at least 99%. Preferably the compounds of the present invention have the “S” configuration at position 1. Also preferred are compounds that have the “R” configuration at position 2. Most preferred are compounds that have the “1S,2R” configuration.

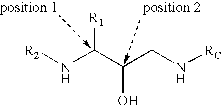

All compound names were generated using AutoNom (AUTOmatic NOMenclature) version 2.1, ACD Namepro version 5.09, Chemdraw Ultra (versions 6.0, 8.0, 8.03, and 9.0), or were derived therefrom.

Several of the compounds of formula (I) are amines, and as such form salts when reacted with acids. Pharmaceutically acceptable salts are preferred over the corresponding amines since they produce compounds, which are more water soluble, stable and/or more crystalline.

### Example 40

EXEMPLARY FORMULA (I) COMPOUNDS

| Example No. | Compound |
|---|---|
| 40.1. | 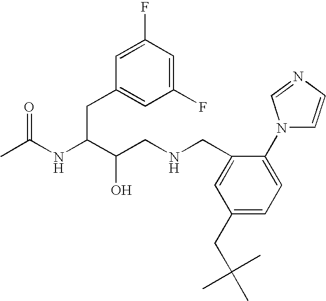 |
| | N-(4-(2-(1H-imidazol-1-yl)-5-neopentylbenzylamino)-1-(3,5- |
| | difluorophenyl)-3-hydroxybutan-2-yl)acetamide |
| 40.2. | 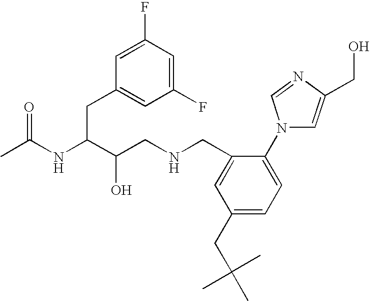 |
| | N-(1-(3,5-difluorophenyl)-3-hydroxy-4-(2-(4-(hydroxymethyl)-1H- |
| | imidazol-1-yl)-5-neopentylbenzylamino)butan-2-yl)acetamide |
| 40.3. | 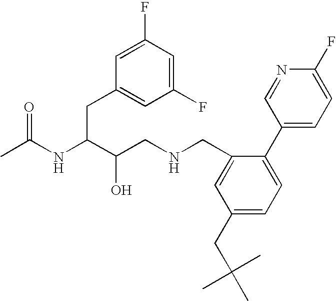 |
| | N-(1-(3,5-difluorophenyl)-4-(2-(6-fluoropyridin-3-yl)-5- |
| | neopentylbenzylamino)-3-hydroxybutan-2-yl)acetamide |
| 40.4. | 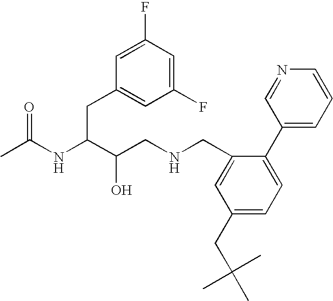 |
| | N-(1-(3,5-difluorophenyl)-3-hydroxy-4-(5-neopentyl-2-(pyridin-3- |
| | yl)benzylamino)butan-2-yl)acetamide |
| 40.5. | 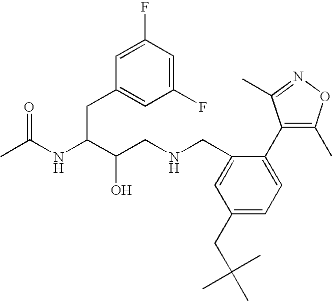 |
| | N-(1-(3,5-difluorophenyl)-4-(2-(3,5-dimethylisoxazol-4-yl)-5- |
| | neopentylbenzylamino)-3-hydroxybutan-2-yl)acetamide |
| 40.6. | 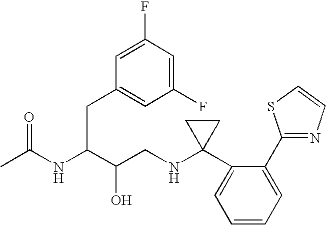 |
| | N-(1-(3,5-difluorophenyl)-3-hydroxy-4-(1-(2-(thiazol-2- |
| | yl)phenyl)cyolopropylamino)butan-2-yl)acetamide |
| 40.7. | 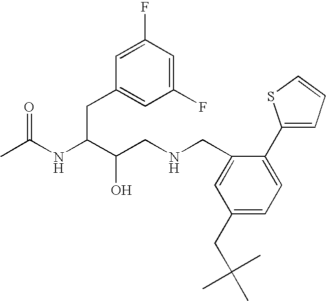 |
| | N-(1-(3,5-difluorophenyl)-3-hydroxy-4-(5-neopentyl-2-(thiophen-2- |
| | yl)benzylamino)butan-2-yl)acetamide |
| 40.8. | 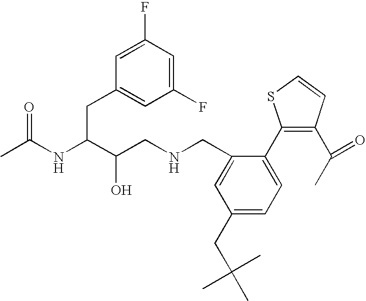 |
| | N-(4-(2-(3-acetylthiophen-2-yl)-5-neopentylbenzylamino)-1-(3,5- |
| | difluorophenyl)-3-hydroxybutan-2-yl)acetamide |
| 40.9. | 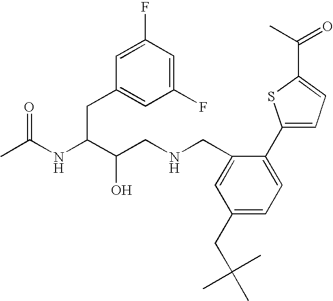 |
| | N-(4-(2-(5-acetylthiophen-2-yl)-5-neopentylbenzylamino)-1-(3,5- |
| | difluorophenyl)-3-hydroxybutan-2-yl)acetamide |
| 40.10. | 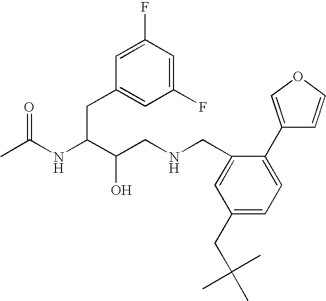 |
| | N-(1-(3,5-difluorophenyl)-4-(2-(furan-2-yl)-5- |
| | neopentylbenzylamino)-3-hydroxybutan-2-yl)acetamide |
| 40.11. | 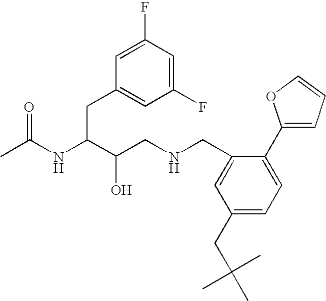 |
| | N-(1-(3,5-difluorophenyl)-4-(2-(furan-2-yl)-5- |
| | neopentylbenzylamino)-3-hydroxybutan-2-yl)acetamide |
| 40.12. | 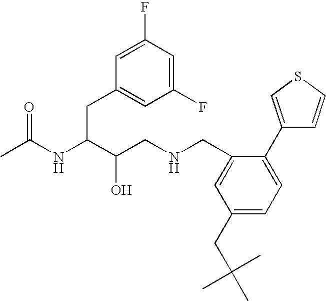 |
| | N-(1-(3,5-difluorophenyl)-3-hydroxy-4-(5-neopentyl-2-(thiophen-3- |
| | yl)benzylamino)butan-2-yl)acetamide |
| 40.13. | 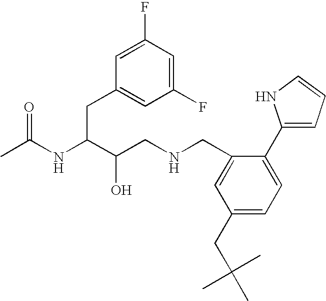 |
| | N-(1-(3,5-difluorophenyl)-3-hydroxy-4-(5-neopentyl-2-(1H-pyrrol-2- |
| | yl)benzylamino)butan-2-yl)acetamide |
| 40.14. | 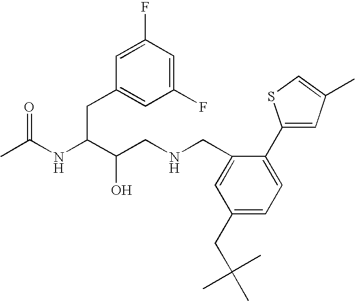 |
| | N-(1-(3,5-difluorophenyl)-3-hydroxy-4-(2-(4-methylthiophen-2-yl)- |
| | 5-neopentylbenzylamino)butan-2-yl)acetamide |
| 40.15. | 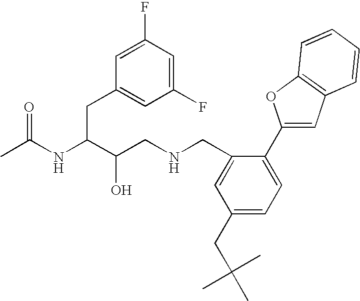 |
| | N-(4-(2-(benzofuran-2-yl)-5-neopentylbenzylamino)-1-(3,5- |
| | difluorophenyl)-3-hydroxybutan-2-yl)acetamide |
| 40.16. | 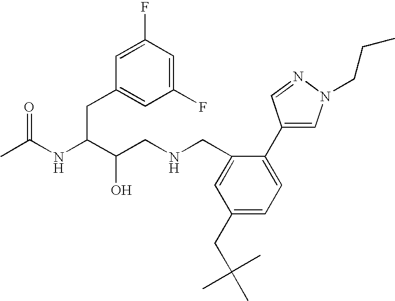 |
| | N-(1-(3,5-difluorophenyl)-3-hydroxy-4-(5-neopentyl-2-(1-propyl-1H- |
| | pyrazol-4-yl)benzylamino)butan-2-yl)acetamide |
| 40.17. | 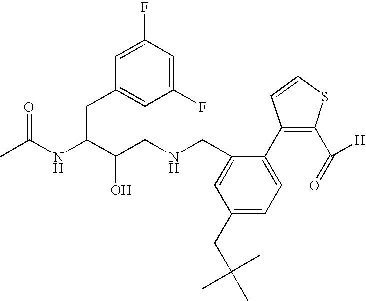 |
| | N-(1-(3,5-difluorophenyl)-4-(2-(2-formylthiophen-3-yl)-5- |
| | neopentylbenzylamino)-3-hydroxybutan-2-yl)acetamide |
| 40.18. | 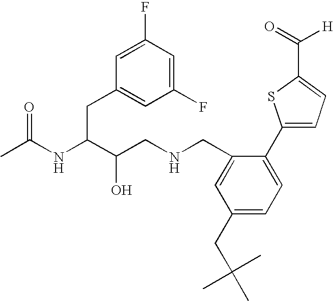 |
| | N-(1-(3,5-difluorophenyl)-4-(2-(5-formylthiophen-2-yl)-5- |
| | neopentylbenzylamino)-3-hydroxybutan-2-yl)acetamide |
| 40.19. | 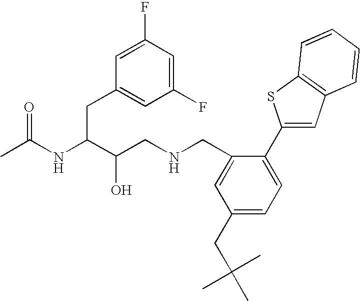 |
| | N-(4-(2-(benzo[b]thiophen-2-yl)-5-neopentylbenzylamino)-1-(3,5- |
| | difluorophenyl)-3-hydroxybutan-2-yl)acetamide |
| 40.20. | 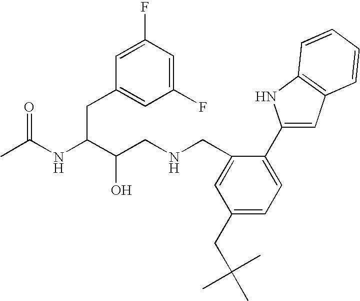 |
| | N-(4-(2-(1H-indol-2-yl)-5-neopentylbenzylamino)-1-(3,5- |
| | difluorophenyl)-3-hydroxybutan-2-yl)acetamide |
| 40.21. | 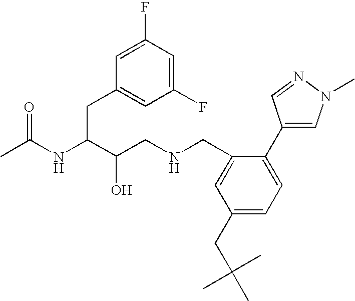 |
| | N-(1-(3,5-difluorophenyl)-3-hydroxy-4-(2-(1-methyl-1H-pyrazol-4- |
| | yl)-5-neopentylbenzylamino)butan-2-yl)acetamide |
| 40.22. | 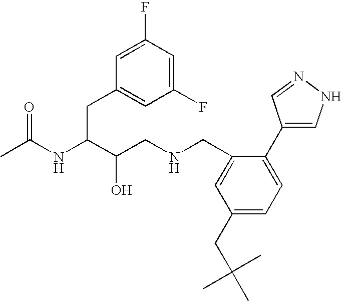 |
| | N-(1-(3,5-difluorophenyl)-3-hydroxy-4-(5-neopentyl-2-(1H-pyrazol |
| | 4-yl)benzylamino)butan-2-yl)acetamide |
| 40.23. | 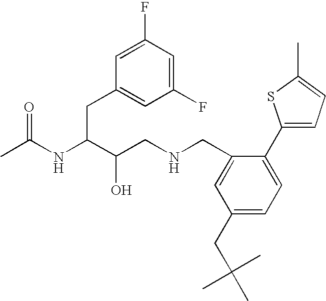 |
| | N-(1-(3,5-difluorophenyl)-3-hydroxy-4-(2-(5-methylthiophen-2-yl)- |
| | 5-neopentylbenzylamino)butan-2-yl)acetamide |
| 40.24. | 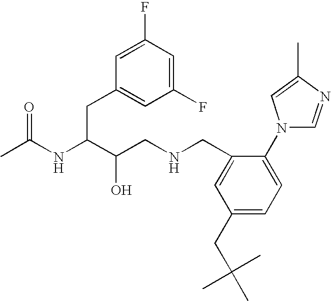 |
| | N-(1-(3,5-difluorophenyl)-3-hydroxy-4-(2-(4-methyl-1H-imidazol-1- |
| | yl)-5-neopentylbenzylamino)butan-2-yl)acetamide |
| 40.25. | 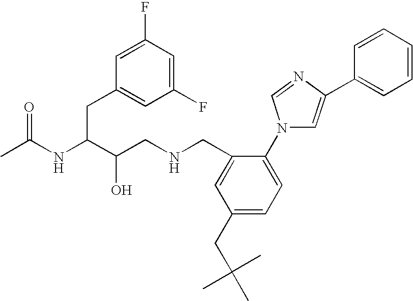 |
| | N-(1-(3,5-difluorophenyl)-3-hydroxy-4-(5-neopentyl-2-(4-phenyl- |
| | 1H-imidazol-1-yl)benzylamino)butan-2-yl)acetamide |
| 40.26. | 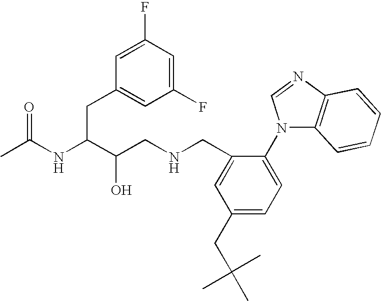 |
| | N-(4-(2-(1H-benzo[d]imidazol-1-yl)-5-neopentylbenzylamino)-1- |
| | (3,5-difluorophenyl)-3-hydroxybutan-2-yl)acetamide |
| 40.27. | 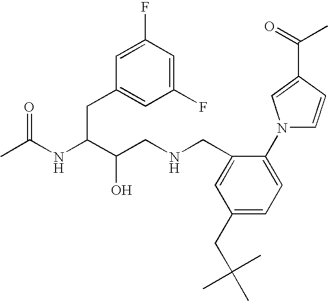 |
| | N-(4-(2-(3-acetyl-1H-pyrrol-1-yl)-5-neopentylbenzylamino)-1-(3,5- |
| | difluorophenyl)-3-hydroxybutan-2-yl)acetamide |
| 40.28. | 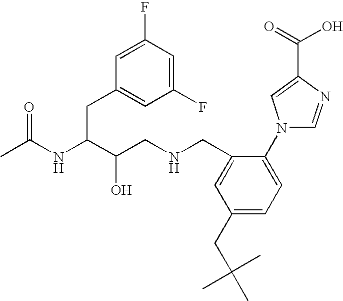 |
| | 1-(2-((3-aoetamido-4-(3,5-difluorophenyl)-2- |
| | hydroxybutylamino)methyl)-4-neopentylphenyl)-1H-imidazole-4- |
| | carboxylic acid |
| 40.29. | 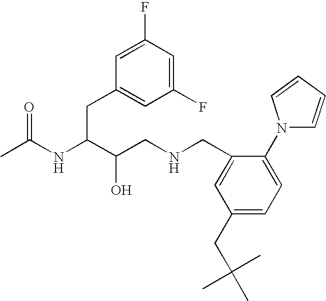 |
| | N-{1-(3,5-Difluoro-benzyl)-3-[5-(2,2-dimethyl-propyl)-2-indol-1-yl- |
| | benzylamino]-2-hydroxy-propyl}-acetamide |
| 40.30. | 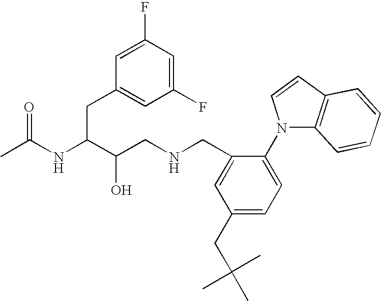 |
| | N-(4-(2-(1H-indol-1-yl)-5-neopentylbenzylamino)-1-(3,5- |
| | difluorophenyl-3-hdroxybutan-2-yl)acetamide |
| 40.31. | 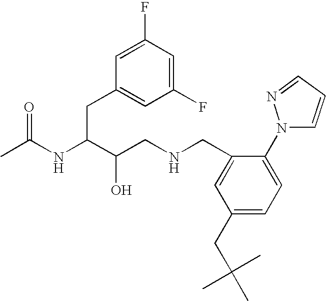 |
| | N-(1-(3,5-difluorophenyl)-3-hydroxy-4-(5-neopentyl-2-(1H-pyrazol- |
| | 1-yl)benzylamino)butan-2-yl)acetamide |
| 40.32. | 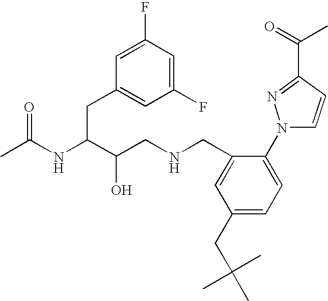 |
| | N-(4-(2-(3-acetyl-1H-pyrazol-1-yl)-5-neopentylbenzylamino)-1- |
| | (3,5-difluorophenyl)-3-hydroxybutan-2-yl)acetamide |
| 40.33. | 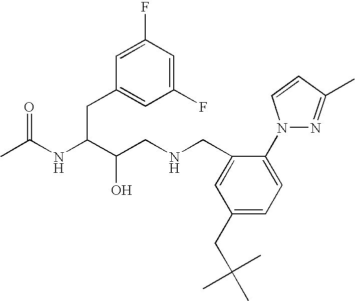 |
| | N-(1-(3,5-difluorophenyl)-3-hydroxy-4-(2-(3-methyl-1H-pyrazol-1- |
| | yl)-5-neopentylbenzylamino)butan-2-yl)acetamide |
| 40.34. | 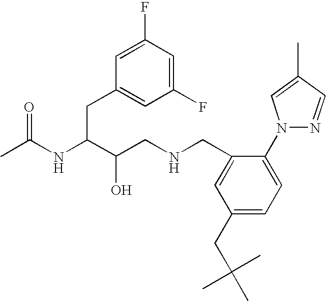 |
| | N-{1-(3,5-Difluoro-benzyl)-3-[5-(2,2-dimethyl-propyl)-2-(4-methyl- |
| | pyrazol-1-yl)-benzylamino]-2-hydroxy-propyl)-acetamide |
| 40.35. | 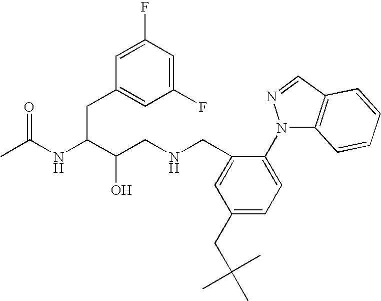 |
| | N-(4-(2-(1H-indazol-1-yl)-5-neopentylbenzylamino)-1-(3,5- |
| | difluorophenyl)-3-hydroxybutan-2-yl)acetamide |
| 40.36. | 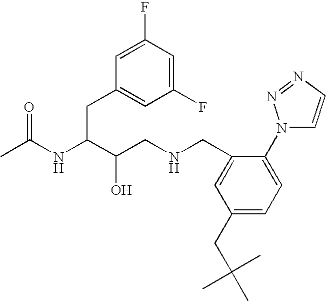 |
| | N-(1-(3,5-difluorophenyl)-3-hydroxy-4-(5-neopentyl-2-(1H-1,2,3- |
| | triazol-1-yl)benzylamino)butan-2-yl)acetamide |
| 40.37. | 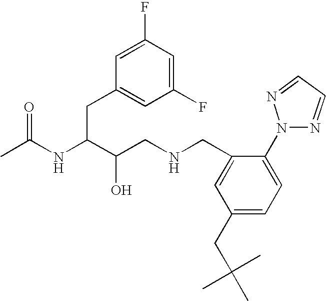 |
| | N-(1-(3,5-difluorophenyl)-3-hydroxy-4-(5-neopentyl-2-(2H-1,2,3- |
| | triazol-2-yl)benzylamino)butan-2-yl)acetamide |
| 40.38. | 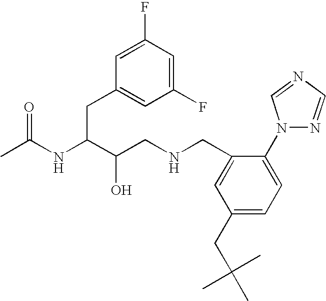 |
| | N-(1-(3,5-difluorophenyl)-3-hydroxy-4-(5-neopentyl-2-(1H-1,2,4- |
| | triazol-1-yl)benzylamino)butan-2-yl)acetamide |
| 40.39. | 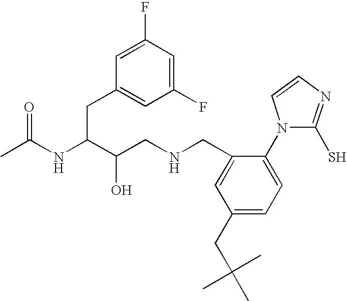 |
| | N-(1-(3,5-difluorophenyl)-3-hydroxy-4-(2-(2-mercapto-1H-imidazol- |
| | 1-yl)-5-neopentylbenzylarnino)butan-2-yl)acetamide |
| 40.40. | 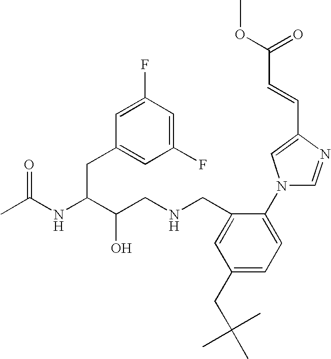 |
| | methyl 3-(1-(2-((3-acetamido-4-(3,5-difluorophenyl)-2- |
| | hydroxybutylamino)methyl)-4-neopentylphenyl)-1H-imidazol-4- |
| | yl)acrylate |
| 40.41. | 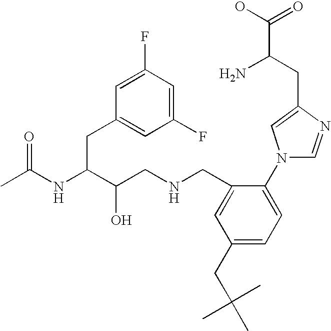 |
| | 3-(1-(2-((3-acetamido-4-(3,5-difluorophenyl)-2- |
| | hydroxybutylamino)methyl)-4-neopentylphenyl)-1H-imidazol-4-yl)- |
| | 2-aminopropanoic acid |
| 40.42. | 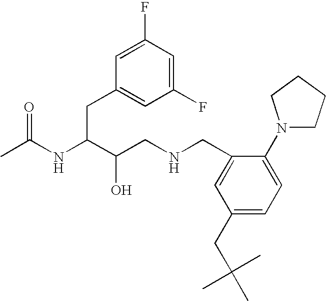 |
| | N-(1-(3,5-difluorophenyl)-3-hydroxy-4-(5-neopentyl-2-(pyrrolidin-1- |
| | yl)benzylamino)butan-2-yl)acetamide |
| 40.43. | 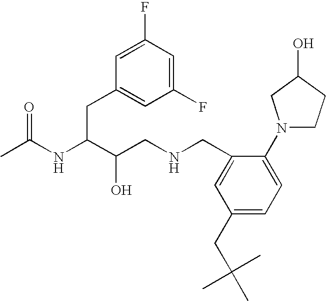 |
| | N-(1-(3,5-difluorophenyl)-3-hydroxy-4-(2-(3-hydroxypyrrolidin-1-yl)- |
| | 5-neopentylbenzylamino)butan-2-yl)acetamide |
| 40.44. | 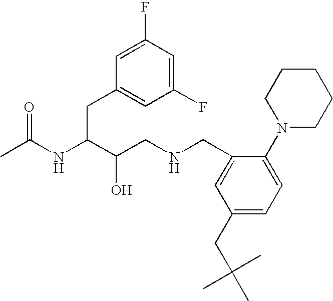 |
| | N-(1-(3,5-difluorophenyl)-3-hydroxy-4-(5-neopentyl-2-(piperidin-1- |
| | yl)benzylamino)butan-2-yl)acetamide |

### Example 41

### BIOLOGICAL EXAMPLES

Properties such as efficacy, oral bioavailability, selectivity, or blood-brain barrier penetration can be assessed by techniques and assays known to one skilled in the art. Exemplary assays for determining such properties are found below.

### INHIBITION OF APP CLEAVAGE

The methods of treatment and compounds of the present invention inhibit cleavage of APP between Met595 and Asp596 numbered for the APP695 isoform, or a mutant thereof, or at a corresponding site of a different isoform, such as APP751 or APP770, or a mutant thereof (sometimes referred to as the “beta secretase site”). While many theories exist, inhibition of beta-secretase activity is thought to inhibit production of A-beta.

Inhibitory activity is demonstrated in one of a variety of inhibition assays, whereby cleavage of an APP substrate in the presence of beta-secretase enzyme is analyzed in the presence of the inhibitory compound, under conditions normally sufficient to result in cleavage at the beta-secretase cleavage site. Reduction of APP cleavage at the beta-secretase cleavage site compared with an untreated or inactive control is correlated with inhibitory activity. Assay systems that can be used to demonstrate efficacy of the compounds of formula (I) are known. Representative assay systems are described, for example, in U.S. Pat. Nos. 5,942,400 and 5,744,346, as well as in the Examples below.

The enzymatic activity of beta-secretase and the production of A-beta can be analyzed in vitro or in vivo, using natural, mutated, and/or synthetic APP substrates, natural, mutated, and/or synthetic enzyme, and the compound employed in the particular method of treatment. The analysis can involve primary or secondary cells expressing native, mutant, and/or synthetic APP and enzyme, animal models expressing native APP and enzyme, or can utilize transgenic animal models expressing the substrate and enzyme. Detection of enzymatic activity can be by analysis of at least one of the cleavage products, for example, by immunoassay, fluorometric or chromogenic assay, HPLC, or other means of detection. Inhibitory compounds are determined as those able to decrease the amount of beta-secretase cleavage product produced in comparison to a control, where beta-secretase mediated cleavage in the reaction system is observed and measured in the absence of inhibitory compounds.

Efficacy reflects a preference for a target tissue. For example, efficacy values yield information regarding a compound's preference for a target tissue by comparing the compound's effect on multiple (i.e., two) tissues. See, for example, Dovey et al., J. Neurochemistry, 2001, 76:173-181. Efficacy reflects the ability of compounds to target a specific tissue and create the desired result (e.g., clinically). Efficacious compositions and corresponding methods of treatment are needed to prevent or treat conditions and diseases associated with amyloidosis.

Efficacious compounds of the present invention are those able to decrease the amount of A-beta produced compared to a control, where beta-secretase mediated cleavage is observed and measured in the absence of the compounds. Detection of efficacy can be by analysis of A-beta levels, for example, by immunoassay, fluorometric or chromogenic assay, HPLC, or other means of detection. The efficacy of the compounds of formula (I) was determined as a percentage inhibition corresponding to A-beta concentrations for tissue treated and untreated with a compound of formula (I).

### BETA-SECRETASE

Various forms of beta-secretase enzyme are known, are available, and useful for assaying enzymatic activity and inhibition of enzyme activity. These include native, recombinant, and synthetic forms of the enzyme. Human beta-secretase is known as Beta Site APP Cleaving Enzyme (BACE), BACE1, Asp2, and memapsin 2, and has been characterized, for example, in U.S. Pat. No. 5,744,346 and published PCT patent applications WO 98/22597, WO 00/03819, WO-01/23533, and WO 00/17369, as well as in literature publications (Hussain et al., 1999, Mol. Cell. Neurosci., 14:419-427; Vassar et al., 1999, Science, 286:735-741; Yan et al., 1999, Nature, 402:533-537; Sinha et al., 1999, Nature, 40:537-540; and Lin et al., 2000, Proceedings Natl. Acad. Sciences USA, 97:1456-1460). Synthetic forms of the enzyme have also been described in, for example, WO 98/22597 and WO 00/17369. Beta-secretase can be extracted and purified from human brain tissue and can be produced in cells, for example mammalian cells expressing recombinant enzyme.

### APP SUBSTRATE

Assays that demonstrate inhibition of beta-secretase-mediated cleavage of APP can utilize any of the known forms of APP, including the 695 amino acid “normal” isotype described by Kang et al., 1987, Nature, 325:733-6, the 770 amino acid isotype described by Kitaguchi et. al., 1981, Nature, 331:530-532, and variants such as the Swedish Mutation (KM670-1NL) (APP-SW), the London Mutation (V7176F), and others. See, for example, U.S. Pat. No. 5,766,846 and also Hardy, 1992, Nature Genet. 1:233-234, for a review of known variant mutations. Additional useful substrates include the dibasic amino acid modification, APP-KK, disclosed, for example, in WO 00/17369, fragments of APP, and synthetic peptides containing the beta-secretase cleavage site, wild type (WT) or mutated form, (e.g., SW), as described, for example, in U.S. Pat. No. 5,942,400 and WO 00/03819.

The APP substrate contains the beta-secretase cleavage site of APP (KM-DA or NL-DA) for example, a complete APP peptide or variant, an APP fragment, a recombinant or synthetic APP, or a fusion peptide. Preferably, the fusion peptide includes the beta-secretase cleavage site fused to a peptide having a moiety useful for enzymatic assay, for example, having isolation and/or detection properties. A useful moiety can be an antigenic epitope for antibody binding, a label or other detection moiety, a binding substrate, and the like.

### ANTIBODIES

Products characteristic of APP cleavage can be measured by immunoassay using various antibodies, as described, for example, in Pirttila et al., 1999, Neuro. Lett., 249:21-4, and in U.S. Pat. No. 5,612,486. Useful antibodies to detect A-beta include, for example, the monoclonal antibody 6E10 (Senetek, St. Louis, Mo.) that specifically recognizes an epitope on amino acids 1-16 of the A-beta peptide; antibodies 162 and 164 (New York State Institute for Basic Research, Staten Island, N.Y.) that are specific for human A-beta 1-40 and 1-42, respectively; and antibodies that recognize the junction region of A-beta, the site between residues 16 and 17, as described in U.S. Pat. No. 5,593,846. Antibodies raised against a synthetic peptide of residues 591 to 596 of APP and SW192 antibody raised against 590-596 of the Swedish mutation are also useful in immunoassay of APP and its cleavage products, as described in U.S. Pat. Nos. 5,604,102 and 5,721,130.

### ASSAY SYSTEMS

Assays for determining APP cleavage at the beta-secretase cleavage site are well known in the art. Exemplary assays, are described, for example, in U.S. Pat. Nos. 5,744,346 and 5,942,400, and described in the Examples below.

### CELL FREE ASSAYS

Exemplary assays that can be used to demonstrate the inhibitory activity of the compounds of the present invention are described, for example, in WO 00/17369, WO 00/03819, and U.S. Pat. Nos. 5,942,400 and 5,744,346. Such assays can be performed in cell-free incubations or in cellular incubations using cells expressing A-beta-secretase and an APP substrate having A-beta-secretase cleavage site.

An APP substrate containing the beta-secretase cleavage site of APP, for example, a complete APP or variant, an APP fragment, or a recombinant or synthetic APP substrate containing the amino acid sequence KM-DA or NL-DA is incubated in the presence of beta-secretase enzyme, a fragment thereof, or a synthetic or recombinant polypeptide variant having beta-secretase activity and effective to cleave the beta-secretase cleavage site of APP, under incubation conditions suitable for the cleavage activity of the enzyme. Suitable substrates optionally include derivatives that can be fusion proteins or peptides that contain the substrate peptide and a modification useful to facilitate the purification or detection of the peptide or its beta-secretase cleavage products. Useful modifications include the insertion of a known antigenic epitope for antibody binding; the linking of a label or detectable moiety, the linking of a binding substrate, and the like.

Suitable incubation conditions for a cell-free in vitro assay include, for example, approximately 200 nM to 10 μM substrate, approximately 10 pM to 200 pM enzyme, and approximately 0.1 nM to 10 μM inhibitor compound, in aqueous solution, at an approximate pH of 4-7, at approximately 37° C., for a time period of approximately 10 min to 3 h. These incubation conditions are exemplary only, and can vary as required for the particular assay components and/or desired measurement system. Optimization of the incubation conditions for the particular assay components should account for the specific beta-secretase enzyme used and its pH optimum, any additional enzymes and/or markers that might be used in the assay, and the like. Such optimization is routine and will not require undue experimentation.

One useful assay utilizes a fusion peptide having maltose binding protein (MBP) fused to the C-terminal 125 amino acids of APP-SW. The MBP portion is captured on an assay substrate by an anti-MBP capture antibody. Incubation of the captured fusion protein in the presence of beta-secretase results in cleavage of the substrate at the beta-secretase cleavage site. Analysis of the cleavage activity can be, for example, by immunoassay of cleavage products. One such immunoassay detects a unique epitope exposed at the carboxy terminus of the cleaved fusion protein, for example, using the antibody SW192. This assay is described, for example, in U.S. Pat. No. 5,942,400.

### CELLULAR ASSAY

Numerous cell-based assays can be used to analyze beta-secretase activity and/or processing of APP to release A-beta. Contact of an APP substrate with A-beta-secretase enzyme within the cell and in the presence or absence of a compound of formula (I) can be used to demonstrate beta-secretase inhibitory activity of the compound. It is preferred that the assay in the presence of a useful inhibitory compound provides at least about 10% inhibition of the enzymatic activity, as compared with a non-inhibited control.

In an embodiment, cells that naturally express beta-secretase are used. Alternatively, cells are modified to express a recombinant beta-secretase or synthetic variant enzyme as discussed above. The APP substrate can be added to the culture medium and is preferably expressed in the cells. Cells that naturally express APP, variant or mutant forms of APP, or cells transformed to express an isoform of APP, mutant or variant APP, recombinant or synthetic APP, APP fragment, or synthetic APP peptide or fusion protein containing the beta-secretase APP cleavage site can be used, provided that the expressed APP is permitted to contact the enzyme and enzymatic cleavage activity can be analyzed.

Human cell lines that normally process A-beta from APP provide useful means to assay inhibitory activities of the compounds employed in the methods of treatment of the present invention. Production and release of A-beta and/or other cleavage products into the culture medium can be measured, for example by immunoassay, such as Western blot or enzyme-linked immunoassay (EIA) such as by ELISA.

Cells expressing an APP substrate and an active beta-secretase can be incubated in the presence of a compound of formula (I) to demonstrate inhibition of enzymatic activity as compared with a control. Activity of beta-secretase can be measured by analysis of at least one cleavage product of the APP substrate. For example, inhibition of beta-secretase activity against the substrate APP would be expected to decrease the release of specific beta-secretase induced APP cleavage products such as A-beta.

Although both neural and non-neural cells process and release A-beta, levels of endogenous beta-secretase activity are low and often difficult to detect by EIA. The use of cell types known to have enhanced beta-secretase activity, enhanced processing of APP to A-beta, and/or enhanced production of A-beta are therefore preferred. For example, transfection of cells with the Swedish Mutant form of APP (APP-SW); with APP-KK; or with APP-SW-KK provides cells having enhanced beta-secretase activity and producing amounts of A-beta that can be readily measured.

In such assays, for example, the cells expressing APP and beta-secretase are incubated in a culture medium under conditions suitable for beta-secretase enzymatic activity at its cleavage site on the APP substrate. On exposure of the cells to the compound of formula (I) employed in the methods of treatment, the amount of A-beta released into the medium and/or the amount of CTF99 fragments of APP in the cell lysates is reduced as compared with the control. The cleavage products of APP can be analyzed, for example, by immune reactions with specific antibodies, as discussed above.

Preferred cells for analysis of beta-secretase activity include primary human neuronal cells, primary transgenic animal neuronal cells where the transgene is APP, and other cells such as those of a stable 293 cell line expressing APP, for example, APP-SW.

### IN VIVO ASSAYS: ANIMAL MODELS

Various animal models can be used to analyze beta-secretase-activity and/or processing of APP to release A-beta, as described above. For example, transgenic animals expressing APP substrate and beta-secretase enzyme can be used to demonstrate inhibitory activity of the compounds of the present invention. Certain transgenic animal models have been described, for example, in. U.S. Pat. Nos. 5,877,399, 5,612,486, 5,387,742, 5,720,936, 5,850,003, 5,877,015, and 5,811,633, and in Games et al., 1995, Nature, 373:523. Animals that exhibit characteristics associated with the pathophysiology of Alzheimer's disease are preferred. Administration of the compounds of the present invention to the transgenic mice described herein provides an alternative method for demonstrating the inhibitory activity of the compounds. Administration of the compounds of the present invention in a pharmaceutically effective carrier and via an administrative route that reaches the target tissue in an appropriate therapeutic amount is also preferred.

Inhibition of beta-secretase mediated cleavage of APP at the beta-secretase cleavage site and of A-beta release can be analyzed in these animals by measuring cleavage fragments in the animal's body fluids such as cerebral fluid or tissues. Analysis of brain tissues for A-beta deposits or plaques is preferred.

### A: Enzyme Inhibition Assay

The methods of treatment and compounds of the present invention are analyzed for inhibitory activity by use of the MBP-C125 assay. This assay determines the relative inhibition of beta-secretase cleavage of a model APP substrate, MBP-C125SW, by the compounds assayed as compared with an untreated control. A detailed description of the assay parameters can be found, for example, in U.S. Pat. No. 5,942,400. Briefly, the substrate is a fusion peptide formed of MBP and the carboxy terminal 125 amino acids of APP-SW, the Swedish mutation. The beta-secretase enzyme is derived from human brain tissue as described in Sinha et al., 1999, Nature, 40:537-540 or recombinantly produced as the full-length enzyme (amino acids 1-501), and can be prepared, for example, from 293 cells expressing the recombinant cDNA, as described in WO 00/47618.

Inhibition of the enzyme is analyzed, for example, by immunoassay of the enzyme's cleavage products. One exemplary ELISA uses an anti-MBP capture antibody that is deposited on precoated and blocked 96-well high binding plates, followed by incubation with diluted enzyme reaction supernatant, incubation with a specific reporter antibody, for example, biotinylated anti-SW192 reporter antibody, and further incubation with streptavidin/alkaline phosphatase. In the assay, cleavage of the intact MBP-C125SW fusion protein results in the generation of a truncated amino-terminal fragment, exposing a new SW-192 antibody-positive epitope at the carboxy terminus. Detection is effected by a fluorescent substrate signal on cleavage by the phosphatase. ELISA only detects cleavage following Leu596 at the substrate's APP-SW 751 mutation site.

### SPECIFIC ASSAY PROCEDURE

Compounds of formula (I) are diluted in a 1:1 dilution series to a six-point concentration curve (two wells per concentration) in one row of a 96-well plate per compound tested. Each of the test compounds is prepared in DMSO to make up a 10 mM stock solution. The stock solution is serially diluted in DMSO to obtain a final compound concentration of 200 μM at the high point of a 6-point dilution curve. 10 μL of each dilution is added to each of two wells on row C of a corresponding V-bottom plate to which 190 μL of 52 mM NaOAc, 7.9% DMSO, pH 4.5 are pre-added. The NaOAc diluted compound plate is spun down to pellet precipitant and 20 μL/well is transferred to a corresponding flat-bottom plate to which 30 μL of ice-cold enzyme-substrate mixture (2.5 μL MBP-C125SW substrate, 0.03 μL enzyme and 24.5 μL ice cold 0.09% TX100 per 30 μL) is added. The final reaction mixture of 200 μM compound at the highest curve point is in 5% DMSO, 20 μM NaOAc, 0.06% TX100, at pH 4.5.

Warming the plates to 37° C. starts the enzyme reaction. After 90 min at 37° C., 200 μL/well cold specimen diluent is added to stop the reaction and 20 μL/well was transferred to a corresponding anti-MBP antibody coated ELISA plate for capture, containing 80 μL/well specimen diluent. This reaction is incubated overnight at 4° C. and the ELISA is developed the next day after a 2 h incubation with anti-192SW antibody, followed by Streptavidin-AP conjugate and fluorescent substrate. The signal is read on a fluorescent plate reader.

Relative compound inhibition potency is determined by calculating the concentration of compound that showed a 50% reduction in detected signal (IC₅₀) compared to the enzyme reaction signal in the control wells with no added compound. In this assay, preferred compounds of the present invention exhibit an IC₅₀ of less than 50 μM.

### B: FP BACE ASSAY: Cell Free Inhibition Assay Utilizing a Synthetic APP Substrate

A synthetic APP substrate that can be cleaved by beta-secretase and having N-terminal biotin and made fluorescent by the covalent attachment of Oregon green at the Cys residue is used to assay beta-secretase activity in the presence or absence of the inhibitory compounds employed in the present invention. Useful substrates include
Biotin-SEVNL-DAEFRC[oregon green]KK,
Biotin-SEVKM-DAEFRC[oregon green]KK,
Biotin-GLNIKTEEISEISY-EVEFRC[oregon green]KK,
Biotin-ADRGLTTRPGSGLTN IKTEEISEVNL-DAEFRC[oregon green]KK, and
Biotin-FVNQHLCoxGSHLVEALY-LVCoxG ERG FFYTPKAC[oregon green]KK.

The enzyme (0.1 nM) and test compounds (0.001-100 μM) are incubated in pre-blocked, low affinity, black plates (384 well) at 37° C. for 30 min. The reaction is initiated by addition of 150 μM substrate to a final volume of 30 μL/well. The final assay conditions are 0.001-100 μM compound of formula (I), 0.1 M sodium acetate (pH 4.5), 150 nM substrate, 0.1 nM soluble beta-secretase, 0.001% Tween 20, and 2% DMSO. The assay mixture is incubated for 3 h at 37° C., and the reaction is terminated by the addition of a saturating concentration of immunopure streptavidin. After incubation with streptavidin at room temperature for 15 min, fluorescence polarization is measured, for example, using a LJL Acqurest (Ex485 nm/Em530 nm).

The activity of the beta-secretase enzyme is detected by changes in the fluorescence polarization that occur when the substrate is cleaved by the enzyme. Incubation in the presence or absence of a compound of formula (I) demonstrates specific inhibition of beta-secretase enzymatic cleavage of its synthetic APP substrate. In this assay, preferred compounds of the present invention exhibit an IC₅₀ of less than 50 μM. More preferred compounds of the present invention exhibit an IC₅₀ of less than 10 μM. Even more preferred compounds of the present invention exhibit an IC₅₀ of less than 5 μM.

### C: Beta-Secretase Inhibition: P26-P4′SW Assay

Synthetic substrates containing the beta-secretase cleavage site of APP are used to assay beta-secretase activity, using the methods described, for example, in published PCT application WO 00/47618. The P26-P4′SW substrate is a peptide of the sequence (biotin)CGGADRGLTTRPGSGLTNIKTEEISEVNLDAEF. The P26-P1 standard has the sequence (biotin)CGGADRGLTTRPGSGLTNIKTEEISEVNL.

Briefly, the biotin-coupled synthetic substrates are incubated at a concentration of from about 0 μM to about 200 μM in this assay. When testing inhibitory compounds, a substrate concentration of about 1.0 μM is preferred. Test compounds diluted in DMSO are added to the reaction mixture, with a final DMSO concentration of 5%. Controls also contain a final DMSO concentration of 5%. The concentration of beta secretase enzyme in the reaction is varied, to give product concentrations with the linear range of the ELISA assay, about 125 μM to 2000 μM, after dilution.

The reaction mixture also includes 20 mM sodium acetate, pH 4.5, 0.06% Triton X100, and is incubated at 37° C. for about 1 to 3 h. Samples are then diluted in assay buffer (for example, 145.4 nM NaCl, 9.51 mM sodium phosphate, 7.7 mM sodium azide, 0.05% Triton X405, 6 g/L bovine serum albumin, pH 7.4) to quench the reaction, then diluted further for immunoassay of the cleavage products.

Cleavage products can be assayed by ELISA. Diluted samples and standards are incubated in assay plates coated with capture antibody, for example, SW192, for about 24 h at 4° C. After washing in TTBS buffer (150 mM NaCl, 25 mM Tris, 0.05% Tween 20, pH 7.5), the samples are incubated with streptavidin-AP according to the manufacturer's instructions. After a 1 h incubation at room temperature, the samples are washed in TTBS and incubated with fluorescent substrate solution A (31.2 g/L 2-amino-2-methyl-1-propanol, 30 mg/L, pH 9.5). Reaction with streptavidin-alkaline phosphate permits detection by fluorescence. Compounds that are effective inhibitors of beta-secretase activity demonstrate reduced cleavage of the substrate as compared to a control.

### D: Assays Using Synthetic Oligopeptide-Substrates

Synthetic oligopeptides are prepared incorporating the known cleavage site of beta-secretase, and optionally include detectable tags, such as fluorescent or chromogenic moieties. Examples of such peptides, as well as their production and detection methods, are described in U.S. Pat. No. 5,942,400. Cleavage products can be detected using high performance liquid chromatography, or fluorescent or chromogenic detection methods appropriate to the peptide to be detected, according to methods well known in the art.

By way of example, one such peptide has the sequence SEVNL-DAEF, and the cleavage site is between residues 5 and 6. Another preferred substrate has the sequence ADRGLTTRPGSGLTNIKTEEISEVNL-DAEF, and the cleavage site is between residues 26 and 27.

These synthetic APP substrates are incubated in the presence of beta-secretase under conditions sufficient to result in beta-secretase mediated cleavage of the substrate. Comparison of the cleavage results in the presence of a compound of formula (I) to control results provides a measure of the compound's inhibitory activity.

### E: Inhibition of Beta-Secretase Activity-Cellular Assay

An exemplary assay for the analysis of inhibition of beta-secretase activity utilizes the human embryonic kidney cell line HEKp293 (ATCC Accession No. CRL-1573) transfected with APP751 containing the naturally occurring double mutation Lys651Met652 to Asn651Leu652 (numbered for APP751), commonly called the Swedish mutation and shown to overproduce A-beta (Citron et al., 1992, Nature, 360:672-674), as described in U.S. Pat. No. 5,604,102.

The cells are incubated in the presence/absence of the inhibitory compound (diluted in DMSO) at the desired concentration, generally up to 10 μg/mL. At the end of the treatment period, conditioned media is analyzed for beta-secretase activity, for example, by analysis of cleavage fragments. A-beta can be analyzed by immunoassay, using specific detection antibodies. The enzymatic activity is measured in the presence and absence of the compound of formula (I) to demonstrate specific inhibition of beta-secretase mediated cleavage of APP substrate.

### F: Inhibition of Beta-Secretase in Animal Models of Alzheimer's Disease

Various animal models can be used to screen for inhibition of beta-secretase activity. Examples of animal models useful in the present invention include mouse, guinea pig, dog, and the like. The animals used can be wild type, transgenic, or knockout models. In addition, mammalian models can express mutations in APP, such as APP695-SW and the like described herein. Examples of transgenic non-human mammalian models are described in U.S. Pat. Nos. 5,604,102, 5,912,410 and 5,811,633.

PDAPP mice, prepared as described in Games et al., 1995, Nature, 373:523-527, are useful to analyze in vivo suppression of A-beta release in the presence of putative inhibitory compounds. As described in U.S. Pat. No. 6,191,166,4-month-old PDAPP mice are administered a compound of formula (I) formulated in a vehicle, such as corn oil. The mice are dosed with the compound (1-30 mg/mL, preferably 1-10 mg/mL). After a designated time, e.g., 3-10 h, the brains are analyzed.

Transgenic animals are administered an amount of a compound formulated in a carrier suitable for the chosen mode of administration. Control animals are untreated, treated with vehicle, or treated with an inactive compound. Administration can be acute, (i.e. single dose or multiple doses in one day), or can be chronic, (i.e. dosing is repeated daily for a period of days). Beginning at time 0, brain tissue or cerebral fluid is obtained from selected animals and analyzed for the presence of APP cleavage peptides, including A-beta, for example, by immunoassay using specific antibodies for A-beta detection. At the end of the test period, animals are sacrificed and brain tissue or cerebral fluid is analyzed for the presence of A-beta and/or beta-amyloid plaques. The tissue is also analyzed for necrosis.

Reduction of A-beta in brain tissues or cerebral fluids and reduction of beta-amyloid plaques in brain tissue are assessed by administering the compounds of formula (I), or pharmaceutical compositions comprising compounds of formula (I) to animals and comparing the data with that from non-treated controls.

### G: Inhibition of A-beta Production in Human Patients

Patients suffering from Alzheimer's disease demonstrate an increased amount of A-beta in the brain. Alzheimer's disease patients are subjected to a method of treatment of the present invention, (i.e. administration of an amount of the compound of formula (I) formulated in a carrier suitable for the chosen mode of administration). Administration is repeated daily for the duration of the test period. Beginning on day 0, cognitive and memory tests are performed, for example, once per month.

Patients administered the compounds of formula (I) are expected to demonstrate slowing or stabilization of disease progression as analyzed by a change in at least one of the following disease parameters: A-beta present in cerebrospinal fluid or plasma; brain or hippocampal volume; A-beta deposits in the brain; amyloid plaque in the brain; or scores for cognitive and memory function, as compared with control, non-treated patients.

### H: Prevention of A-beta Production in Patients at Risk for Alzheimer's Disease

Patients predisposed or at risk for developing Alzheimer's disease can be identified either by recognition of a familial inheritance pattern, for example, presence of the Swedish Mutation, and/or by monitoring diagnostic parameters. Patients identified as predisposed or at risk for developing Alzheimer's disease are administered an amount of the compound of formula (I) formulated in a carrier suitable for the chosen mode of administration. Administration is repeated daily for the duration of the test period. Beginning on day 0, cognitive and memory tests are performed, for example, once per month.

Patients subjected to a method of treatment of the present invention (i.e., administration of a compound of formula (I)) are expected to demonstrate slowing or stabilization of disease progression as analyzed by a change in at least one of the following disease parameters: A-beta present in cerebrospinal fluid or plasma; brain or hippocampal volume; amyloid plaque in the brain; or scores for cognitive and memory function, as compared with control, non-treated patients.

### I: Efficacy of Compounds to Inhibit A-beta Concentration

The invention encompasses compounds of formula (I) that are efficacious. Efficacy is calculated as a percentage of concentrations as follows:
Efficacy=(1−(total A-beta in dose group/total A-beta in vehicle control))*100%
wherein the “total A-beta in dose group” equals the concentration of A-beta in the tissue, (e.g., rat brain) treated with the compound, and the “total A-beta in vehicle control” equals the concentration of A-beta in the tissue, yielding a % inhibition of A-beta production. Statistical significance is determined by p-value <0.05 using the Mann Whitney t-test. See, for example, Dovey et al., J. Neurochemistry, 2001, 76:173-181.

Where indicated, diastereomers were separated by reverse phase HPLC using the noted methods. The first isomer collected in each case was designated Diastereomer A, and the second isomer Diastereomer B. Where indicated, specific formula (I) compound examples represent single diastereomers (e.g., diastereomer A).

Efficacy For Exemplary Formula (I) Compound

| | | Efficacy | |
|---|---|---|---|
| | | (% Inhibition, | |
| Example | | 100 mg/kg) | |
| No. | Compound | cortex | plasma |
| 41.1 | 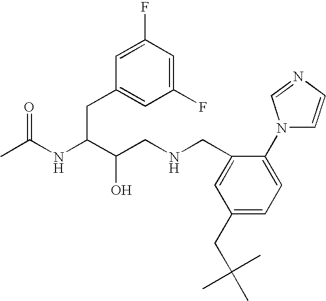 | 15 | 47 |
| | N-{1-(3,5-Difluoro-benzyl)-3-[5-(2,2- | | |
| | dimethyl-propyl)-2-imidazol-1-yl- | | |
| | benzylamino]-2-hydroxy-propyl}-acetamide | | |

### J: Selectivity of Compounds for Inhibiting BACE over Aspartyl Proteases

The compounds of formula (I) can be selective for beta-secretase versus catD. Wherein the ratio of catD:beta-secretase is greater than 1, selectivity is calculated as follows:
Selectivity=(IC₅₀ for catD/IC₅₀ for beta-secretase)*100%
wherein IC₅₀ is the concentration of compound necessary to decrease the level of catD or beta-secretase by 50%. Selectivity is reported as the ratio of IC₅₀(catD):IC₅₀(BACE).

The compounds of formula (I) can be selective for beta-secretase versus catE. Wherein the ratio of catE:beta-secretase is greater than 1, selectivity is calculated as follows:
Selectivity=(IC₅₀ for catE IC₅₀ for beta-secretase)*100%
wherein IC₅₀ is the concentration of compound necessary to decrease the level of catE or beta-secretase by 50%. Selectivity is reported as the ratio of IC₅₀(catE):IC₅₀(BACE).

Pharmacokinetic parameters were calculated by a non-compartmental approach. See, for example, Gibaldi, M. and Perrier, D., Pharmacokinetics, Second Edition, 1982, Marcel Dekker Inc., New York, N.Y., pp 409-418.

In the following examples, each value is an average of four experimental runs and multiple values for one compound indicate that more than one experiment was conducted.

Selectivity For BACE Versus catD of Exemplary Formula (I) Compound

| | | Selectivity |
|---|---|---|
| Example | | IC₅₀(catD)/ |
| No. | Compound | IC₅₀(BACE) |
| 41.2 | 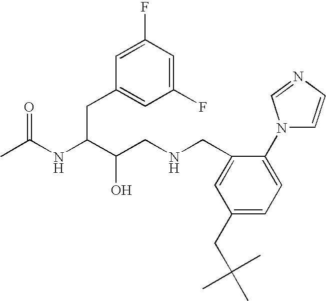 | 41.8 5.8 3.5 |
| | N-(4-(2-(1H-imidazol-1-yl)-5- | |
| | neopentylbenzylamino)-1-(3,5-difluorophenyl)-3- | |
| | hydroxybutan-2-yl)acetamide | |
| 41.3 | 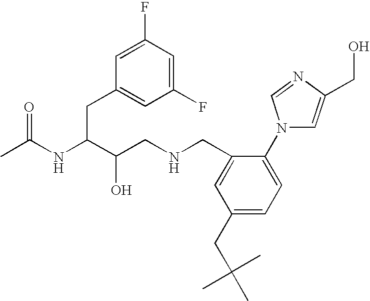 | 1.6 |
| | N-(1-(3,5-difluorophenyl)-3-hydroxy-4-(2-(4- | |
| | (hydroxymethyl)-1H-imidazol-1-yl)-5- | |
| | neopentylbenzylamino)butan-2-yl)acetamide | |
| 41.4 | 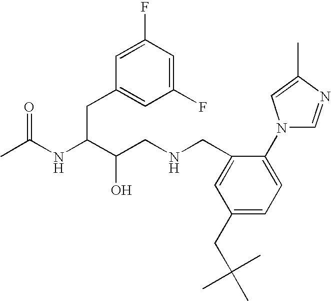 | 1.7 |
| | N-{1-(3,5-Difluoro-benzyl)-3-[5-(2,2-dimethyl- | |
| | propyl)-2-(4-methyl-imidazol-1-yl)-benzylamino]-2- | |
| | hydroxy-propyl}-acetamide | |
| 41.5 | 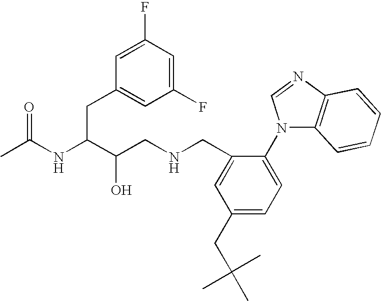 | 3.6 3.0 |
| | N-[3-[2-Benzoimidazol-1-yl-5-(2,2-dimethyl-propyl)- | |
| | benzylamino]-1-(3,5-difluoro-benzyl)-2-hydroxy- | |
| | propyl]-acetamide | |
| 41.6 | 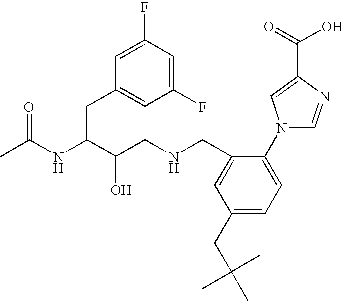 | 35.4 2.3 |
| | 1-[2-{[3-Acetylamino-4-(3,5-difluoro-phenyl)-2- | |
| | hydroxy-butylamino]-methyl}-4-(2,2-dimethyl- | |
| | propyl)-phenyl]-1H-imidazole-4-carboxylic acid | |
| 41.7 | 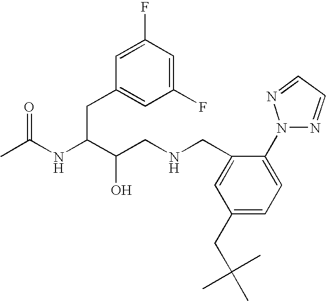 | >33.1 |
| | N-(1-(3,5-difluorophenyl)-3-hydroxy-4-(5-neopentyl- | |
| | 2-(2H-1,2,3-triazol-2-yl)benzylamino)butan-2- | |
| | yl)acetamide | |
| 41.8 | 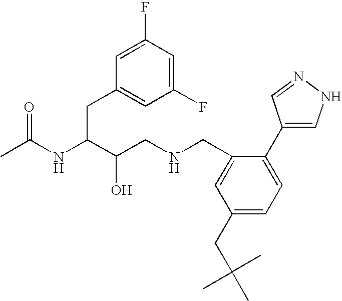 | 11.73.6 |
| | N-(1-(3,5-difluorophenyl)-3-hydroxy-4-(5-neopentyl- | |
| | 2-(1H-pyrazol-4-yl)benzylamino)butan-2- | |
| | yl)acetamide | |

Selectivity For BACE Versus catE of Exemplary Formula (I) Compound

| | | Selectivity |
|---|---|---|
| Example | | IC₅₀(catE)/ |
| No. | Compound | IC₅₀(BACE) |
| 41.8 | 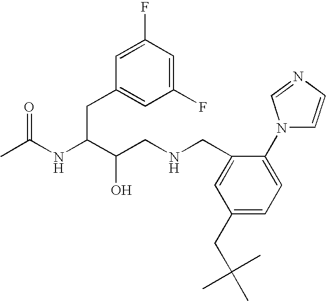 | 9.0 2.8 |
| | N-(4-(2-(1H-imidazol-1-yl)-5- | |
| | neopentylbenzylamino)-1-(3,5-difluorophenyl)-3- | |
| | hydroxybutan-2-yl)acetamide | |
| 41.9 | 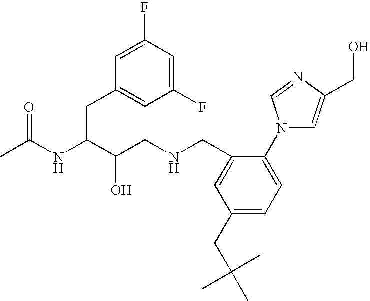 | 1.2 |
| | N-(1-(3,5-difluorophenyl)-3-hydroxy-4-(2-(4- | |
| | (hydroxymethyl)-1H-imidazol-1-yl)-5- | |
| | neopentylbenzylamino)butan-2-yl)acetamide | |
| 41.10 | 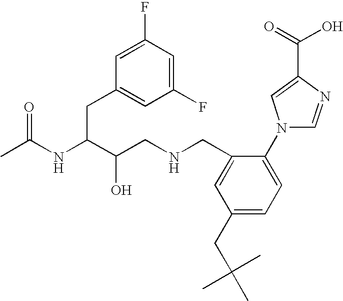 | 11.9 2.4 |
| | 1-[2-{[3-Acetylamino-4-(3,5-difluoro-phenyl)-2- | |
| | hydroxy-butylamino]-methyl}-4-(2,2-dimethyl- | |
| | propyl)-phenyl]-1H-imidazole-4-carboxylic acid | |
| 41.11 | 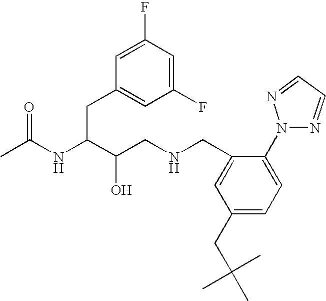 | 1.4 |
| | N-(1-(3,5-difluorophenyl)-3-hydroxy-4-(5-neopentyl- | |
| | 2-(2H-1,2,3-triazol-2-yl)benzylamino)butan-2- | |
| | yl)acetamide | |
| 41.12 | 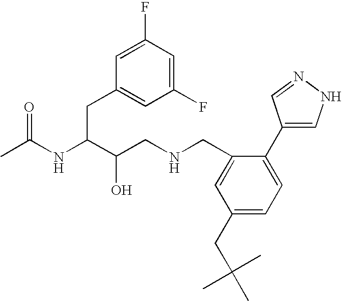 | 4.7 |
| | N-(1-(3,5-difluorophenyl)-3-hydroxy-4-(5-neopentyl- | |
| | 2-(1H-pyrazol-4-yl)benzylamino)butan-2- | |
| | yl)acetamide | |

### K: Oral Bioavailability of Compounds for Inhibiting Amyloidosis

The invention encompasses compounds of formula (I) that are orally bioavailable. Generally, oral bioavailability is defined as the fraction of orally administered dose reaching systemic circulation. Oral bioavailability can be determined following both an intravenous (IV) and oral (PO) administration of a test compound.

Oral bioavailability was determined in the male Sprague-Dawley rat following both IV and PO administration of test compound. Two month-old male rats (250-300 g) were surgically implanted with polyethylene (PE-50) cannula in the jugular vein while under isoflurane anesthesia the day before the in-life phase. Animals were fasted overnight with water ad libitum, then dosed the next day. The dosing regime consisted of either a 5 mg/kg (2.5 mL/kg) IV dose (N=3) administered to the jugular vein cannula, then flushed with saline, or a 10 mg/kg (5 mL/kg) PO dose (N=3) by esophageal gavage. Compounds were formulated with 10% Solutol in 5% dextrose at 2 mg/mL. Subsequent to dosing, blood was collected at 0.016 (IV only), 0.083, 0.25, 0.5, 1, 3, 6, 9, and 24 h post administration, and heparinized plasma was recovered following centrifugation.

Compounds were extracted from samples following precipitation of the plasma proteins by methanol. The resulting supernatants were evaporated to dryness and reconstituted with chromatographic mobile phase (35% acetonitrile in 0.1% formic acid) and injected onto a reverse phase C₁₈ column (2×50 mm, 5 μm, BDS Hypersil). Detection was facilitated with a multi-reaction-monitoring experiment on a tandem triple quadrupole mass spectrometer (LC/MS/MS) following electrospray ionization. Experimental samples were compared to calibration curves prepared in parallel with aged match rat plasma and quantitated with a weighted 1/x linear regression. The lower limit of quantization (LOQ) for the assay was typically 0.5 ng/mL.

Oral bioavailability (% F) is calculated from the dose normalized ratio of plasma exposure following oral administration to the intravenous plasma exposure in the rat by the following equation
% F=(AUCₚₒ/AUCᵢᵥ)×(Dᵢᵥ/Dₚₒ)×100%
where D is the dose and AUC is the area-under-the-plasma-concentration-time-curve from 0 to 24 h. AUC is calculated from the linear trapezoidal rule by AUC=((C₂+C₁)/2)×(T₂−T₁) where C is concentration and T is time.

Pharmacokinetic parameters were calculated by a non-compartmental approach. See, for example, Gibaldi, M. and Perrier, D., Pharmacokinetics, Second Edition, 1982, Marcel Dekker Inc., New York, N.Y., pp 409-418.

Oral Bioavailability For Exemplary Formula (I) Compound

| Example | | |
|---|---|---|
| No. | Compound | % F |
| 41.12 | 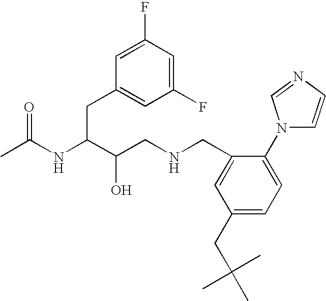 | 15 |
| | N-(4-(2-(1H-imidazol-1-yl)-5-neopentylbenzylamino)- | |
| | 1-(3,5-difluorophenyl)-3-hydroxybutan-2- | |
| | yl)acetamide | |

### L: Brain Uptake

The invention encompasses beta-secretase inhibitors that can readily cross the blood-brain barrier. Factors that affect a compound's ability to cross the blood-brain barrier include a compound's molecular weight, Total Polar Surface Area (TPSA), and log P (lipophilicity). See, e.g., Lipinski, C. A., et al., Adv. Drug Deliv. Reviews, 23:3-25 (1997). One of ordinary skill in the art will be aware of methods for determining characteristics allowing a compound to cross the blood-brain barrier. See, for example, Murcko et al., Designing Libraries with CNS Activity, J. Med. Chem., 42 (24), pp. 4942-51 (1999).

Calculations of logP values were performed using the Daylight clogP program (Daylight Chemical Information Systems, Inc.). See, for example, Hansch, C., et al., Substituent Constants for Correlation Analysis in Chemistry and Biology, Wiley, New York (1979); Rekker, R., The Hydrophobic Fragmental Constant, Elsevier, Amsterdam (1977); Fujita, T., et al., J. Am. Chem. Soc., 86, 5157 (1964). TPSA was calculated according to the methodology outlined in Ertl, P., et al., J. Med. Chem., 43:3714-17 (2000).

The following assay was employed to determine the brain penetration of compounds encompassed by the present invention.

In-life phase: Test compounds were administered to CF-1 (20-30 g) mice at 10 μmol/kg (4 to 7 mg/kg) following IV administration in the tail vein. Two time-points, 5 and 60 min, were collected post dose. Four mice were harvested for heparinized plasma and non-perfused brains at each time-point for a total of 8 mice per compound.

Analytical phase: Samples were extracted and evaporated to dryness, then reconstituted and injected onto a reverse phase chromatographic column while monitoring the effluent with a triple quadrupole mass spectrometer. Quantitation was then performed with a 1/x² weighted fit of the least-squares regression from calibration standards prepared in parallel with the in vivo samples. The LOQ is generally 1 ng/mL and 0.5 ng/g for the plasma and brain respectively. Data was reported in micromolar (μM) units. Brain levels were corrected for plasma volumes (16 μL/g).

Results: Comparison of a compound's brain concentration level to two marker compounds, Indinavir and Diazepam, demonstrates the ability in which the compounds of the present invention can cross the blood-brain barrier. Indinavir (HIV protease inhibitor) is a poor brain penetrant marker and Diazepam is a blood flow limited marker. The concentration levels of Indinavir in the brain at 5 and 60 min were 0.165 μM and 0.011 μM, respectively. The concentration levels of Diazepam at 5 and 60 min were 5.481 μM and 0.176 μM, respectively.

The present invention has been described with reference to various specific and preferred embodiments and techniques. However, it should be understood that many variations and modifications may be made while remaining within the spirit and scope of the present invention.

Unless defined otherwise, all scientific and technical terms used herein have the same meaning as commonly understood by one of skill in the art to which this invention belongs. Although methods and materials similar or equivalent to those described herein can be used in the practice or testing of the present invention, suitable methods and materials are described above. Additionally, the materials, methods, and examples are illustrative only and not intended to be limiting. All publications, patent applications, patents, and other references mentioned herein are incorporated by reference in their entirety. In case of conflict, the present specification, including definitions, will control.

What is claimed is:

1. A method of preventing or treating at least one condition which benefits from inhibition of at least one aspartyl-protease, comprising:
administering to a host in need thereof a composition comprising a therapeutically effective amount of at least one compound of formula (I), 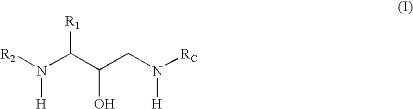
or a pharmaceutically acceptable salt thereof, wherein
R₁ is selected from 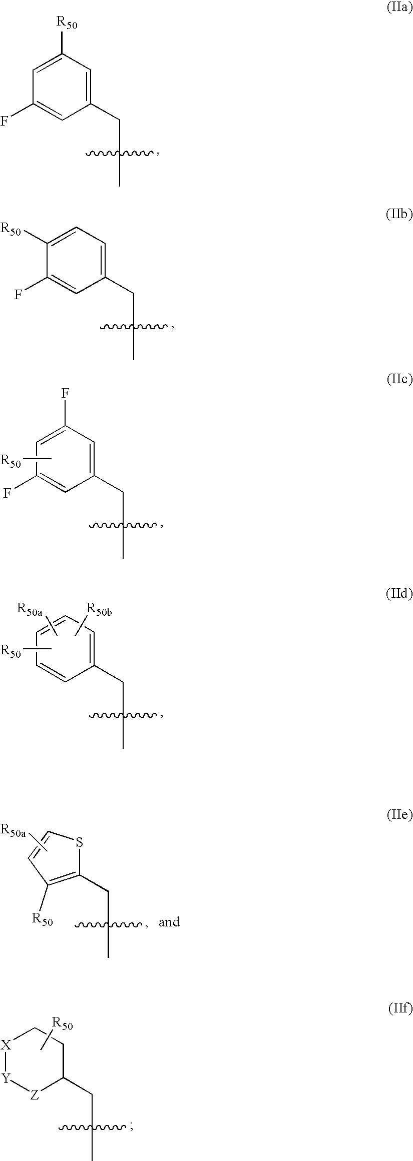
wherein;
X, Y, and Z are independently selected from
—C(H)₀₋₂—,
—O—,
—C(O)—,
—NH—, and
—N—,
wherein at least one bond of the (IIf) ring may optionally be a double bond;
R₅₀, R₅₀ₐ, and R_{50b} are independently selected from
—H,
-halogen,
—OH,
—SH,
—CN,
—C(O)-alkyl,
—NR₇R₈,
—S(O)₀₋₂-alkyl,
-alkyl,
-alkoxy,
—O-benzyl optionally substituted with at least one substituent independently selected from —H, —OH, and alkyl,
—C(O)—NR₇R₈,
-alkyloxy,
-alkoxyalkoxyalkoxy, and
-cycloalkyl;
wherein the alkyl, alkoxy, and cycloalkyl groups within R₅₀, R₅₀ₐ, and R_{50b} are optionally substituted with at least one substituent independently selected from alkyl, halogen, —OH, —NR₅R₆, —NR₇R₈, —CN, haloalkoxy, and alkoxy;
R₅ and R₆ are independently selected from —H and alkyl; or
R₅ and R₆, and the nitrogen to which they are attached, form a 5 or 6-membered heterocycloalkyl ring; and
R₇ and R₈ are independently selected from
—H,
-alkyl optionally substituted with at least one group independently selected from —OH, —NH₂, and halogen,
-cycloalkyl, and
-alkyl-O-alkyl;
R₂ is selected from —C(O)—CH₃, —C(O)—CH₂(halogen), —C(O)—CH(halogen)₂, 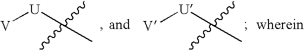
U is selected from —C(O)—, —C(═S)—, —S(O)₀₋₂—, —C═N—R₂₁—, —C═N—OR₂₁—, —C(O)—NR₂₀—, —C(O)—O—, —S(O)₂—NR₂₀—, and —S(O)₂—O—;
U′ is selected from —C(O)—, —C═N—R₂₁—, —C═N—OR₂₁—, —C(O)—NR₂₀—, and —C(O)—O—;
V is selected from aryl, heteroaryl, cycloalkyl, heterocycloalkyl, —[C(R₄)(R₄)]₁1₃-D, and -(T)₀₋₁-R_{N};
V′ is selected from -(T)₀₋₁-R_{N′};
wherein the aryl, heteroaryl, cycloalkyl, and heterocycloalkyl groups included within V and V′ are optionally substituted with 1 or 2 R_{B} groups;
wherein at least one carbon of the aryl, heteroaryl, cycloalkyl, and heterocycloalkyl groups included within V and V′ are optionally replaced with —N—, —O—, —NH—, —C(O)—, —C(S)—, —C(═N—H)—, —C(═N—OH)—, —C(=N-alkyl)-, or —C(═N—O-alkyl)-;
R_{B} at each occurrence is independently selected from halogen, —OH, —CF₃, —OCF₃, —O-aryl, —CN, —NR₁₀₁R′₁₀₁, -alkyl, -alkoxy, —(CH₂)₀₋₄—(C(O))₀₋₁—(O)₀₋₁-alkyl, —C(O)—OH, —(CH₂)₀₋₃-cycloalkyl, -aryl, -heteroaryl, and -heterocycloalkyl;
wherein, the alkyl, alkoxy, cycloalkyl, aryl, heteroaryl, or heterocycloalkyl groups included within R_{B} are optionally substituted with 1 or 2 groups independently selected from —C₁-C₄ alkyl, —C₁-C₄ alkoxy-, —C₁-C₄ haloalkyl, —C₁-C₄ haloalkoxy, -halogen, —OH, —CN, and —NR₁₀₁R′₁₀₁;
R₁₀₁ and R′₁₀₁ are independently selected from —H, -alkyl, —(C(O))₀₋₁—(O)₀₋₁-alkyl, —C(O)—OH, and -aryl;
R₄ and R_{4′} are independently selected from -hydrogen, -alkyl, —(CH₂)₀₋₃-cycloalkyl, —(CH₂)₀₋₃—OH, -fluorine, —CF₃, —OCF₃, —O-aryl, -alkoxy, —C₃-C₇ cycloalkoxy, -aryl, and -heteroaryl, or
R₄ and R_{4′} are taken together with the carbon to which they are attached to form a 3, 4, 5, 6, or 7 membered carbocyclic ring wherein 1, 2, or 3 carbons of the ring is optionally replaced with —O—, —N(H)—, —N(alkyl)-, —N(aryl)-, —C(O)—, or —S(O)₀₋₂;
D is selected from aryl, heteroaryl, cycloalkyl, and heterocycloalkyl, wherein the aryl, heteroaryl, cycloalkyl, and heterocycloalkyl are optionally substituted with 1 or 2 R_{B} groups;
T is selected from —NR₂₀— and —O—;
R₂₀ is selected from H, —CN, -alkyl, -haloalkyl, and -cycloalkyl;
R₂₁-is selected from —H, -alkyl, -haloalkyl, and -cycloalkyl;
R_{N} is selected from —OH, —NH₂, —NH(alkyl), —NH(cycloalkyl), —N(alkyl)(alkyl), —N(alkyl)(cycloalkyl), —N(cycloalkyl)(cycloalkyl), —R′₁₀₀, alkyl-R₁₀₀, —(CRR′)₁₋₆R′₁₀₀, —(CRR′)₀₋₆R₁₀₀, —(CRR′)₁₋₆—O—R′₁₀₀, —(CRR′)₁₋₆—S—R′₁₀₀, —(CRR′)₁₋₆—C(O)—R₁₀₀, —(CRR′)₁₋₆—SO₂—R₁₀₀, and —(CRR′)₁₋₆—NR₁₀₀—R′₁₀₀, —(CRR′)₁₋₆—P(O)(O-alkyl)₂, alkyl-O-alkyl-C(O)OH, and —CH(R_{E1})—(CH₂)₀₋₃-E₁-E₂-E₃;
R_{N′} is —SO₂R′₁₀₀;
R and R′ are independently selected from -hydrogen, —C₁-C₁₀ alkyl (optionally substituted with at least one group independently selected from —OH, —C₁-C₁₀ alkylaryl, and —C₁-C₁₀ alkylheteroaryl);
R₁₀₀ and R′₁₀₀ are independently selected from
-cycloalkyl,
-heterocycloalkyl,
-aryl,
-heteroaryl,
-alkoxy,
-aryl-W-aryl,
-aryl-W-heteroaryl,
-aryl-W-heterocycloalkyl,
-heteroaryl-W-aryl,
-heteroaryl-W-heteroaryl,
-heteroaryl-W-heterocycloalkyl,
-heterocycloalkyl-W-aryl,
-heterocycloalkyl-W-heteroaryl,
-heterocycloalkyl-W-heterocycloalkyl,
—W—R₁₀₂,
—CH[(CH₂)₀₋₂—O—R₁₅₀]—(CH₂)₀₋₂-aryl,
—CH[(CH₂)₀₋₂—O—R₁₅₀]—(CH₂)₀₋₂-cycloalkyl,
—CH[(CH₂)₀₋₂—O—R₁₅₀]—(CH₂)₀₋₂-heterocycloalkyl,
—CH[(CH₂)₀₋₂—O—R₁₅₀]—(CH₂)₀₋₂-heteroaryl,
—C₁-C₁₀ alkyl optionally substituted with 1, 2, or 3 R₁₁₅ groups, wherein 1, 2, or 3 carbons of the alkyl group are optionally replaced with a group independently selected from —C(O)— and —NH—,
-alkyl-O-alkyl optionally substituted with 1, 2, or 3 R₁₁₅ groups,
-alkyl-5-alkyl optionally substituted with 1, 2, or 3 R₁₁₅ groups, and
-cycloalkyl optionally substituted with 1, 2, or 3 R₁₁₅ groups;
wherein the ring portions of each group included within R₁₀₀ and R′₁₀₀ are optionally substituted with 1, 2, or 3 groups independently selected from —OR, —NO₂, -halogen, —CN, —OCF₃, —CF₃, —(CH₂)₀₋₄—O—P(═O)(OR)(OR′), —(CH₂)₀₋₄—C(O)—NR₁₀₅R′₁₀₅, —(CH₂)₀₋₄—O—(CH₂)₀₋₄—C(O)NR₁₀₂R₁₀₂′, —(CH₂)₀₋₄—C(O)—(C₁-C₁₂ alkyl), —(CH₂)₀₋₄—C(O)—(CH₂)₀₋₄—cycloalkyl, —(CH₂)₀₋₄—R₁₁₀, —(CH₂)₀₋₄-R₁₂₀, —(CH₂)₀₋₄—R₁₃₀, —(CH₂)₀₋₄—C(O)—R₁₁₀, —(CH₂)₀₋₄—C(O)—R₁₂₀, —(CH₂)₀₋₄—C(O)—R₁₃₀, —(CH₂)₀₋₄—C(O)—R₁₄₀, —(CH₂)₀₋₄—C(O)—O—R₁₅₀, —(CH₂)₀₋₄—SO₂—NR₁₀₅R′₁₀₅, —(CH₂)₀₋₄—SO—(C₁-C₈ alkyl), —(CH₂)₀₋₄—SO₂—(C₁-C₁₂ alkyl), —(CH₂)₀₋₄—SO₂—(CH₂)₀₋₄-cycloalkyl, —(CH₂)₀₋₄—N(R₁₅₀)—C(O)—O—R₁₅₀, —(CH₂)₀₋₄—N(R₁₅₀)—C(O)—N(R₁₅₀)₂, —(CH₂)₀₋₄—N(R₁₅₀)—CS—N(R₁₅₀)₂, —(CH₂)₀₋₄—N(R₁₅₀)—C(O)—R₁₀₅, —(CH₂)₀₋₄—NR₁₀₅R′₁₀₅, —(CH₂)₀₋₄—R₁₄₀, —(CH₂)₀₋₄—O—C(O)-(alkyl), —(CH₂)₀₋₄—O—P(O)—(O—R₁₁₀)₂, —(CH₂)₀₋₄—O—C(O)—N(R₁₅₀)₂, —(CH₂)₀₋₄—O—CS—N(R₁₅₀)₂, —(CH₂)₀₋₄—O—(R₁₅₀), —(CH₂)₀₋₄—O—R_{150′}—C(O)OH, —(CH₂)₀₋₄—S—(R₁₅₀), —(CH₂)₀₋₄—N(R₁₅₀)—SO₂—R₁₀₅, —(CH₂)₀₋₄-cycloalkyl, and —(C₁-C₁₀)-alkyl;
R_{E1} is selected from —H, —OH, —NH₂, —NH—(CH₂)₀₋₃—R_{E2}, —NHR_{E8}, —NR_{E350}C(O)R_{E5}, —C₁-C₄ alkyl-NHC(O)R_{E5}, —(CH₂)₀₋₄R_{E8}, —O—(C₁-C₄ alkanoyl), —C₆-C₁₀ (aryloxy optionally substituted with 1, 2, or 3 groups that are independently selected from halogen, —C₁-C₄ alkyl, —CO₂H, —C(O)—C₁-C₄ alkoxy, and —C₁-C₄ alkoxy), alkoxy, -aryl-(C₁-C₄ alkoxy), —NR_{E350}CO₂R_{E351}, —C₁-C₄ alkyl-NR_{E350}CO₂R_{E351}, —CN, —CF₃, —CF₂—CF₃, —C≡CH, —CH₂—CH═CH₂, —(CH₂)₁₋₄—R_{E2}, —(CH₂)₁₋₄—NH—R_{E2}, —O—(CH₂)₀₋₃—R_{E2}, —S—(CH₂)₀₋₃—R_{E2}, —(CH₂)₀₋₄—NHC(O)—(CH₂)₀₋₆—R_{E352}, and —(CH₂)₀₋₄—(R_{E353})₀₋₁—(CH₂)₀₋₄—R_{E354};
R_{E2} is selected from —SO₂—(C₁-C₈ alkyl), —SO—(C₁-C₈ alkyl), —S—(C₁-C₈ alkyl), —S—C(O)-alkyl, —SO₂—NR_{E3}R_{E4}, —C(O)—C₁-C₂ alkyl, and —C(O)—NR_{E4}R_{E10};
R_{E3} and R_{E4} are independently selected from —H, —C₁-C₃ alkyl, and —C₃-C₆ cycloalkyl;
R_{E10} is selected from alkyl, arylalkyl, alkanoyl, and arylalkanoyl;
R_{E5} is selected from cycloalkyl, alkyl (optionally substituted with 1, 2, or 3 groups that are independently selected from halogen, —NR_{E6}R_{E7}, C₁-C₄ alkoxy, —C₅-C₆ heterocycloalkyl, —C₅-C₆ heteroaryl, —C₆-C₁₀ aryl, —C₃-C₇ cycloalkyl C₁-C₄ alkyl, —S—C₁-C₄ alkyl, —SO₂—C₁-C₄ alkyl, —CO₂H, —C(O)NR_{E6}R_{E7}, —CO₂—C₁-C₄ alkyl, and —C₆-C₁₀ aryloxy), heteroaryl (optionally substituted with 1, 2, or 3 groups that are independently selected from —C₁-C₄ alkyl, —C₁-C₄ alkoxy, halogen, —C₁-C₄ haloalkyl, and —OH), heterocycloalkyl (optionally substituted with 1, 2, or 3 groups independently selected from —C₁-C₄ alkyl, —C₁-C₄ alkoxy, halogen, and —C₂-C₄ alkanoyl), aryl (optionally substituted with 1, 2, 3, or 4 groups independently selected from halogen, —OH, —C₁-C₄ alkyl, —C₁-C₄ alkoxy, and —C₁-C₄ haloalkyl), and —NR_{E6}R_{E7};
R_{E6} and R_{E7} are independently selected from —H, alkyl, alkanoyl, aryl, —SO₂—C₁-C₄ alkyl, and -aryl-C₁-C₄ alkyl;
R_{E8} is selected from —SO₂-heteroaryl, —SO₂-aryl, —SO₂-heterocycloalkyl, —SO₂—C₁-C₁₀ alkyl, —C(O)NHR_{E9}, heterocycloalkyl, —S— alkyl, and —S—C₂-C₄ alkanoyl;
R_{E9} is selected from H, alkyl, and -aryl C₁-C₄ alkyl;
R_{E350} is selected from H and alkyl;
R_{E351} is selected from alkyl, -aryl-(C₁-C₄ alkyl), alkyl (optionally substituted with 1, 2, or 3 groups independently selected from halogen, cyano, heteroaryl, —NR_{E6}R_{E7}, —C(O)NR_{E6}R_{E7}, —C₃-C₇ cycloalkyl, and —C₁-C₄ alkoxy), heterocycloalkyl (optionally substituted with 1 or 2 groups independently selected from —C₁-C₄ alkyl, —C₁-C₄ alkoxy, halogen, —C₂-C₄ alkanoyl, -aryl-(C₁-C₄ alkyl), and —SO₂—(C₁-C₄ alkyl)), heteroaryl (optionally substituted with 1, 2, or 3 groups independently selected from —OH, —C₁-C₄ alkyl, —C₁-C₄ alkoxy, halogen, —NH₂, —NH(alkyl), and —N(alkyl)(alkyl)), heteroarylalkyl (optionally substituted with 1, 2, or 3 groups independently selected from —C₁-C₄ alkyl, —C₁-C₄ alkoxy, halogen, —NH₂, —NH(alkyl), and —N(alkyl)(alkyl)), aryl, heterocycloalkyl, —C₃-C₈ cycloalkyl, and cycloalkylalkyl;
wherein the aryl, heterocycloalkyl, —C₃-C₈ cycloalkyl, and cycloalkylalkyl groups included within R_{E351} are optionally substituted with 1, 2, 3, 4 or 5 groups independently selected from halogen, —CN, —NO₂, alkyl, alkoxy, alkanoyl, haloalkyl, haloalkoxy, hydroxy, hydroxyalkyl, alkoxyalkyl, —C₁-C₆ thioalkoxy, —C₁-C₆ thioalkoxy-alkyl, and alkoxyalkoxy;
R_{E352} is selected from heterocycloalkyl, heteroaryl, aryl, cycloalkyl, —S(O)₀₋₂-alkyl, —CO₂H, —C(O)NH₂, —C(O)NH(alkyl), —C(O)N(alkyl)(alkyl), —CO₂-alkyl, —NHS(O)₀₋₂-alkyl, —N(alkyl)S(O)₀₋₂-alkyl, —S(O)₀₋₂-heteroaryl, —S(O)₀₋₂-aryl, —NH(arylalkyl), —N(alkyl)(arylalkyl), thioalkoxy, and alkoxy; .
wherein each group included within R₃₅₂ is optionally substituted with 1, 2, 3, 4, or 5 groups that are independently selected from alkyl, alkoxy, thioalkoxy, halogen, haloalkyl, haloalkoxy, alkanoyl, —NO₂, —CN, alkoxycarbonyl, and aminocarbonyl;
R_{E353} is selected from —O—, —C(O)—, —NH—, —N(alkyl)-, —NH—S(O)₀₋₂—, —N(alkyl)-S(O)₀₋₂—S(O)₀₋₂—NH—, —S(O)₀₋₂—N(alkyl)-, —NH—C(S)—, and —N(alkyl)-C(S)—;
R_{E354} is selected from heteroaryl, aryl, arylalkyl, -heterocycloalkyl, —CO₂H, —CO₂-alkyl, —C(O)NH(alkyl), —C(O)N(alkyl)(alkyl), —C(O)NH₂, —C₁-C₈ alkyl, —OH, aryloxy, alkoxy, arylalkoxy, —NH₂, —NH(alkyl), —N(alkyl)(alkyl), and -alkyl-CO₂-alkyl;
wherein each group included within R_{E354} is optionally substituted with 1, 2, 3, 4, or 5 groups that are independently selected from alkyl, alkoxy, —CO₂H, —CO₂-alkyl, thioalkoxy, halogen, haloalkyl, haloalkoxy, hydroxyalkyl, alkanoyl, —NO₂, —CN, alkoxycarbonyl, and aminocarbonyl;
E₁ is selected from —NR_{E11}— and —C₁-C₆ alkyl- (optionally substituted with 1, 2, or 3 groups selected from —C₁-C₄ alkyl), and R_{E11} is selected from —H and alkyl; or R_{E1} and R_{E11} combine to form —(CH₂)₁₋₄—;
E₂ is selected from a bond, —SO₂—, —SO—, —S—, and —C(O)—; and
E₃ is selected from —H, —C₁-C₄ haloalkyl, —C₅-C₆ heterocycloalkyl, —C₆-C₁₀ aryl, —OH, —N(E₃a)(E₃b), —C₁-C₁₀ alkyl (optionally substituted with 1, 2, or 3 groups independently selected from halogen, hydroxy, alkoxy, thioalkoxy, and haloalkoxy), —C₃-C₈ cycloalkyl (optionally substituted with 1, 2, or 3 groups independently selected from —C₁-C₃ alkyl and halogen), alkoxy, aryl (optionally substituted with at least one group selected from halogen, alkyl, alkoxy, —CN and —NO₂), arylalkyl (optionally substituted with a group selected from halogen, alkyl, alkoxy, —CN, and —NO₂);
E₃ₐ and E_{3b} are independently selected from —H, —C₁-C₁₀ alkyl (optionally substituted with 1, 2, or 3 groups independently selected from halogen, —C₁-C₄ alkoxy, —C₃-C₈ cycloalkyl, and —OH), —C₂-C₆ alkyl, —C₂-C₆ alkanoyl, aryl, —SO₂—C₁-C₄ alkyl, -aryl-C₁-C₄ alkyl, and —C₃-C₈ cycloalkyl C₁-C₄ alkyl; or
E₃ₐ, E_{3b}, and the nitrogen to which they are attached may optionally form a ring selected from piperazinyl, piperidinyl, morpholinyl, and pyrrolidinyl;
wherein each ring is optionally substituted with 1, 2, 3, or 4 groups that are independently selected from alkyl, alkoxy, alkoxyalkyl, and halogen;
W is selected from —(CH₂)₀₋₄—, —O—, —S(O)₀₋₂—, —N(R₁₃₅)—, —CR(OH)—, and —C(O)—;
R₁₀₂ and R₁₀₂′ are independently selected from hydrogen, —OH, and —C₁-C₁₀ alkyl optionally substituted with 1, 2, or 3 groups independently selected from -halogen, -aryl, and —R₁₁₀;
R₁₀₅ and R′₁₀₅ are independently selected from
—H,
—R₁₁₀,
—R₁₂₀,
-cycloalkyl,
—(C₁-C₂ alkyl)-cycloalkyl,
-(alkyl)-O—(C₁-C₃ alkyl), and
-alkyl optionally substituted with at least one group independently selected from —OH, -amine, and -halogen; or
R₁₀₅ and R′₁₀₅ together with the atom to which they are attached form a 3, 4, 5, 6, or 7 membered carbocyclic ring, wherein one member is optionally a heteroatom selected from —O—, —S(O)₀₋₂—, and —N(R₁₃₅)—, wherein the carbocyclic ring is optionally substituted with 1, 2 or 3 R₁₄₀ groups; and
wherein the at least one carbon of the carbocyclic ring is optionally replaced with —C(O)—;
R₁₁₀ is aryl optionally substituted with 1 or 2 R₁₂₅ groups;
R₁₁₅ at each occurrence is independently selected from halogen, —OH, —C(O)—O—R₁₀₂, —C₁-C₆ thioalkoxy, —C(O)—O-aryl, —NR₁₀₅R′₁₀₅, —SO₂—(C₁-C₈ alkyl), —C(O)—R₁₈₀, R₁₈₀, —C(O)NR₁₀₅R′₁₀₅, —SO₂NR₁₀₅R′₁₀₅, —NH—C(O)-(alkyl), —NH—C(O)—OH, —NH—C(O)—OR, —NH—C(O)—O-aryl, —O—C(O)-(alkyl), —O—C(O)-amino, —O—C(O)-monoalkylamino, —O—C(O)-dialkylamino, —O—C(O)-aryl, —O-(alkyl)-C(O)—O—H, —NH—SO₂— (alkyl), -alkoxy, and -haloalkoxy;
R₁₂₀ is -heteroaryl, optionally substituted with 1 or 2 R₁₂₅ groups;
R₁₂₅ at each occurrence is independently selected from -halogen, -amino, -monoalkylamino, -dialkylamino, —OH, —CN, —SO₂—NH₂, —SO₂—NH-alkyl, —SO₂—N(alkyl)₂, —SO₂—(C₁-C₄ alkyl), —C(O)—NH₂, -Q(O)—NH-alkyl, —C(O)—N(alkyl)₂, -alkyl optionally substituted with 1, 2, or 3 groups independently selected from C₁-C₃ alkyl, halogen, —OH, —SH, —CN, —CF₃, —C₁-C₃ alkoxy, -amino, -monoalkylamino, and -dialkylamino, and -alkoxy optionally substituted with 1, 2, or 3-halogen;
R₁₃₀ is heterocycloalkyl optionally substituted with 1 or 2 R₁₂₅ groups;
R₁₃₅ is independently selected from alkyl, cycloalkyl, —(CH₂)₀₋₂-(aryl), —(CH₂)₀₋₂-(heteroaryl), and —(CH₂)₀₋₂-(heterocycloalkyl);
R₁₄₀ at each occurrence is independently selected from heterocycloalkyl optionally substituted with 1, 2, 3, or 4 groups independently selected from -alkyl, -alkoxy, -halogen, -hydroxy, -cyano, -nitro, -amino, -monoalkylamino, -dialkylamino, -haloalkyl, -haloalkoxy, -amino-alkyl, -monoalkylamino-alkyl, and -dialkylaminoalkyl; and wherein at least one carbon of the heterocycloalkyl is optionally replaced with —C(O);
R₁₅₀ is independently selected from
-hydrogen,
-cycloalkyl,
—(C₁-C₂ alkyl)-cycloalkyl,
—R₁₁₀,
—R₁₂₀, and
-alkyl optionally substituted with 1, 2, 3, or 4 groups independently selected from —OH, —NH₂, —C₁-C₃ alkoxy, —R₁₁₀, and -halogen;
R₁₅₀′ is independently selected from
-cycloalkyl,
—(C₁-C₃ alkyl)-cycloalkyl,
—R₁₂₀, and
-alkyl optionally substituted with 1, 2, 3, or 4 groups independently selected from —OH, —NH₂, —C₁-C₃ alkoxy, —R₁₁₀, and -halogen; and
R₁₈₀ is independently selected from
-morpholinyl,
-thiomorpholinyl,
-piperazinyl,
-piperidinyl,
-homomorpholinyl,
-homothiomorpholinyl,
-homothiomorpholinyl S-oxide,
-homothiomorpholinyl S,S-dioxide,
-pyrrolinyl, and
-pyrrolidinyl;
wherein each R₁₈₀ is optionally substituted with 1, 2, 3, or 4 groups independently selected from -alkyl, -alkoxy, -halogen, -hydroxy, -cyano, -nitro, -amino, -monoalkylamino, -dialkylamino, -haloalkyl, -haloalkoxy, -aminoalkyl, -monoalkylamino-alkyl, -dialkylamino-alkyl and —C(O); and
wherein at least one carbon of R₁₈₀ is optionally replaced with —C(O)—;
R_{C} is 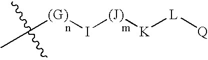
n is 0 or 1;
m is 0 or 1;
G is selected from —C(O)— and —CO₂—;
I is (CH₂)₀₋₄;
J is selected from —(CR₂₄₅R₂₅₀)—;
K is selected from aryl and heteroaryl;
L is selected from
-a bond,
-alkyl- optionally substituted with at least one group independently selected from R₂₀₅,
-(CH₂)₀₋₄—(CO)₀₋₁—N(R₂₂₀)—,
—(CH₂)₀₋₄—(CO)₀₋₁—,
—(CH₂)₀₋₄—CO₂—,
—(CH₂)₀₋₄—SO₂—N(R₂₂₀)—,
—(CH₂)₀₋₄—N(H or R₂₁₅)—CO₂—,
—(CH₂)₀₋₄—N(H or R₂₁₅)—SO₂—,
—(CH₂)₀₋₄—N(H or R₂₁₅)—C(O)—N(R₂₁₅)—,
—(CH₂)₀₋₄—N(H or R₂₁₅)—C(O)—,
—(CH₂)₀₋₄—N(R₂₂₀)—,
—(CH₂)₀₋₄—O—, and
—(CH₂)₀₋₄—S—;
Q is selected from aryl, heteroaryl, cycloalkyl, and heterocycloalkyl;
wherein each cycloalkyl or heterocycloalkyl included within R_{C} is optionally substituted with at least one group independently selected from R₂₀₅;
wherein each aryl or heteroaryl group included within R_{C} is optionally substituted with at least one group independently selected from R₂₀₀;
wherein at least one heteroatom of the heteroaryl group included within R_{C} is optionally substituted with a group independently selected from
—(CO)₀₋₁R₂₁₅,
—(CO)₀₋₁R₂₂₀, and
—S(O)₀₋₂R₂₀₀;
wherein the aryl or heteroaryl group included within R_{C} is optionally substituted with R₂₀₀;
R₂₀₀ at each occurrence is independently selected from:
-alkyl optionally substituted with at least one group independently selected from R₂₀₅,
—OH,
—NO₂,
-halogen,
—CN,
—(CH₂)₀₋₄—C(O)H,
—(CO)₀₋₁R₂₁₅,
—(CO)₀₋₁R₂₂₀,
—(CH₂)₀₋₄—(CO)₀₋₁—N R₂₂₀R₂₂₅,
—(CH₂)₀₋₄—C(O)-alkyl,
—(CH₂)₀₋₄—(CO)₀₋₁-cycloalkyl,
—(CH₂)₀₋₄—(CO)₀₋₁-heterocycloalkyl,
—(CH₂)₀₋₄—(CO)₀₋₁-aryl,
—(CH₂)₀₋₄—(CO)₀₋₁-heteroaryl,
—(CH₂)₀₋₄—CO₂R₂₁₅,
—(CH₂)₀₋₄—SO₂—N R₂₂₀R₂₂₅,
—(CH₂)₀₋₄—S(O)₀₋₂-alkyl,
—(CH₂)₀₋₄—S(O)₀₋₂-cycloalkyl,
—(CH₂)₀₋₄—N(H or R₂₁₅)—CO₂R₂₁₅,
—(CH₂)₀₋₄—N(H or R₂₁₅)—SO₂—R₂₂₀,
—(CH₂)₀₋₄—N(H or R₂₁₅)—C(O)—N(R₂₁₅)₂,
—(CH₂)₀₋₄—N(H or R₂₁₅)—C(O)—R₂₂₀,
—(CH₂)₀₋₄—NR₂₂₀R₂₂₅,
—(CH₂)₀₋₄—O—C(O)-alkyl,
—(CH₂)₀₋₄—O—(R₂₁₅),
—(CH₂)₀₋₄—S—(R₂₁₅),
—(CH₂)₀₋₄—O-alkyl optionally substituted with at least one halogen, and -adamantane;
wherein each aryl and heteroaryl group included within R₂₀₀ is optionally substituted with at least one group independently selected from R₂₀₅, R₂₁₀, and alkyl optionally substituted with at least one group independently selected from R₂₀₅ and R₂₁₀;
wherein each cycloalkyl or heterocycloalkyl group included within R₂₀₀ is optionally substituted with at least one group independently selected from R₂₁₀; R₂₀₅ at each occurrence is independently selected from
-alkyl,
-haloalkoxy,
—(CH₂)₀₋₃-cycloalkyl,
-halogen,
—(CH₂)₀₋₆—OH,
—O-aryl,
—OH,
—SH,
—(CH₂)₀₋₄—C(O)H,
—(CH₂)₀₋₆—CN,
—(CH₂)₀₋₆—C(O)—NR₂₃₅R₂₄₀,
—(CH₂)₀₋₆—C(O)—R₂₃₅,
—(CH₂)₀₋₄—N(H or R₂₁₅)—SO₂—R₂₃₅,
—CF₃,
—CN,
-alkoxy,
-alkoxycarbonyl, and
—NR₂₃₅R₂₄₀;
R₂₁₀ at each occurrence is independently selected from
—OH,
—CN,
—(CH₂)₀₋₄—C(O)H,
-alkyl optionally substituted with at least one group independently selected from R₂₀₅,
—S(O)₂-alkyl,
-halogen,
-alkoxy,
-haloalkoxy,
—NR₂₂₀R₂₂₅,
-cycloalkyl optionally substituted with at least one group independently selected from R₂₀₅,
—C(O)-alkyl,
—S(O)₂—N R₂₃₅R₂₄₀,
—C(O)—NR₂₃₅R₂₄₀, and
—S-alkyl;
R₂₁₅ at each occurrence is independently selected from
-alkyl,
—(CH₂)₀₋₂-cycloalkyl,
—(CH₂)₀₋₂-aryl,
—(CH₂)₀₋₂-heteroaryl,
—(CH₂)₀₋₂-heterocycloalkyl, and
—CO₂—CH₂-aryl;
wherein the aryl groups included within R₂₁₅ are optionally substituted with at least one group independently selected from R₂₀₅ and R₂₁₀,
wherein the heterocycloalkyl and heteroaryl groups included within R₂₁₅ are optionally substituted with at least one group independently selected from R₂₁₀;
R₂₂₀ and R₂₂₅ at each occurrence are independently selected from
—H,
-alkyl,
—(CH₂)₀₋₄—C(O)H,
—(CH₂)₀₋₄—C(O)-alkyl,
-hydroxyalkyl,
-alkoxycarbonyl,
-alkylamino,
—S(O)₂-alkyl,
—C(O)-alkyl optionally substituted with at least one halogen,
—C(O)—NH₂,
—C(O)—NH(alkyl),
—C(O)—N(alkyl)(alkyl),
-haloalkyl,
—(CH₂)₀₋₂-cycloalkyl,
-(alkyl)-O-(alkyl),
-aryl,
-heteroaryl, and
-heterocycloalkyl;
wherein the aryl, heteroaryl and heterocycloalkyl groups included within R₂₂₀ and R₂₂₅ are each optionally substituted with at least one group independently selected from R₂₇₀;
R₂₃₅ and R₂₄₀ at each occurrence are independently selected from
—H,
—OH,
—CF₃,
—OCH₃,
—NH—CH₃,
—N(CH₃)₂,
—(CH₂)₀₋₄—C(O)—(H or alkyl),
-alkyl,
—C(O)-alkyl,
—SO₂-alkyl, and
-aryl;
R₂₄₅ and R₂₅₀ at each occurrence are independently selected from
—H,
—OH,
—(CH₂)₀₋₄CO₂-alkyl,
—(CH₂)₀₋₄C(O)-alkyl,
-alkyl,
-hydroxyalkyl,
-alkoxy,
-haloalkoxy,
—(CH₂)₀₋₄-cycloalkyl,
—(CH₂)₀₋₄-aryl,
—(CH₂)₀₋₄-heteroaryl, and
—(CH₂)₀₋₄-heterocycloalkyl; or
R₂₄₅ and R₂₅₀ are taken together with the carbon to which they are attached to form a monocyclic or bicyclic ring system of 3, 4, 5, 7, or 8 carbon atoms;
wherein at least one carbon atom is optionally replaced by at least one group independently selected from —O—, —S—, —SO₂—, —C(O)—, —NR₂₂₀—, and —N(alkyl)(alkyl); and
wherein the ring is optionally substituted with at least one group independently selected from
-alkyl,
-alkoxy,
—OH,
—NH₂,
—NH(alkyl),
—N(alkyl)(alkyl),
—NH—C(O)-alkyl,
—NH—SO₂-alkyl, and
-halogen;
wherein the aryl, heteroaryl, and heterocycloalkyl groups included within R₂₄₅ and R₂₅₀ are optionally substituted with at least one group independently selected from halogen, alkyl, —CN, and —OH;
R₂₇₀ at each occurrence is independently selected from
—R₂₀₅,
-alkyl optionally substituted with at least one group independently selected from R₂₀₅,
-aryl,
-halogen,
-alkoxy,
-haloalkoxy,
—NR₂₃₅R₂₄₀,
—OH,
—CN,
-cycloalkyl optionally substituted with at least one group independently selected from R₂₀₅,
—C(O)-alkyl,
—S(O)₂—N R₂₃₅R₂₄₀,
—CO—N R₂₃₅R₂₄₀,
—S(O)₂-alkyl, and
—(CH₂)₀₋₄—C(O)H.

2. The method according to claim 1, wherein R₁ is 3,5-difluorobenzyl.

3. The method according to claim 1, wherein R₂ is —C(O)—CH₃.

4. The method according to claim 1, wherein R_{C} is selected from 5-(2,2-dimethyl-propyl)-2-(2-propyl-imidazol-1-yl)-benzyl, 5-(2,2-dimethyl-propyl)-2-(1H-pyrrol-2-yl)-benzyl, 5-(2,2-dimethyl-propyl)-2-(imidazol-1-yl)-benzyl, 5-(2,2-dimethyl-propyl)-2-(1H-pyrazol-4-yl)-benzyl, 5-(2,2-dimethyl-propyl)-2-[1,2,3]thiadiazol-4-yl-benzyl, 5-(2,2-dimethyl-propyl)-2-thiazol-5-yl-benzyl, 5-(2,2-dimethyl-propyl)-2-(3-methyl-isothiazol-5-yl)-benzyl, 5-(2,2-dimethyl-propyl)-2-(2H-[1,2,3] triazol-4-yl)-benzyl, 5-(2,2-dimethyl-propyl)-2-pyridin-3-yl-benzyl, 5-(2,2-dimethyl-propyl)-2-(6-fluoro-pyridin-3-yl)-benzyl, 5-(2,2-dimethyl-propyl)-2-(2-fluoro-pyridin-3-yl)-benzyl, 5-(2,2-dimethyl-propyl)-2-pyridazin-3-yl-benzyl, 5-(2,2-dimethyl-propyl)-2-pyrimidin-5-yl-benzyl, 5-(2,2-dimethyl-propyl)-2-imidazol-1-yl-phenyl]-cyclopropyl, 5-(2,2-dimethyl-propyl)-2-pyrazin-2-yl-benzyl, 5-(2,2-dimethyl-propyl)-2-(5-ethyl-imidazol-1-yl)-benzyl, 3-Chloro-5-(2,2-dimethyl-propyl)-2-imidazol-1-yl-benzyl, 5-(2,2-dimethyl-propyl)-2-tetrazol-1-yl-benzyl, and 5-(2,2-dimethyl-propyl)-2-imidazol-1-yl-benzyl.

5. The method according to claim 1, wherein the at least one compound of formula (I) is chosen from N-{1-(3,5-difluoro-benzyl)-3-[5-(2,2-dimethyl-propyl)-2-(2-propyl-imidazol-1-yl)-benzylamino]-2-hydroxy-propyl}-acetamide, N-(1-(3,5-difluorophenyl)-3-hydroxy-4-(5-neopentyl-2-(1H-pyrrol-2-yl)benzylamino)butan-2-yl)acetamide, N-(4-(2-(1H-imidazol-1-yl)-5-neopentylbenzylamino)-1-(3,5-difluorophenyl)-3-hydroxybutan-2-yl)acetamide N-(4-(2-(1H-imidazol-1-yl)-5-neopentylbenzylamino)-1-(3,5-difluorophenyl)-3-hydroxybutan-2-yl)acetamide, N-{1-(3,5-difluoro-benzyl)-3-[5-(2,2-dimethyl-propyl)-2-(1H-pyrazol-4-yl)-benzylamino]-2-hydroxy-propyl}-acetamide, N-{1-(3,5-difluoro-benzyl)-3-[5-(2,2-dimethyl-propyl)-2-[1,2,3]thiadiazol-4-yl-benzylamino]-2-hydroxy-propyl}-acetamide, N-{1-(3,5-difluoro-benzyl)-3-[5-(2,2-dimethyl-propyl)-2-thiazol-5-yl-benzylamino]-2-hydroxy-propyl}-acetamide, N-{1-(3,5-difluoro-benzyl)-3-[5-(2,2-dimethyl-propyl)-2-(3-methyl-isothiazol-5-yl)-benzylamino]-2-hydroxy-propyl}-acetamide, N-{1-(3,5-difluoro-benzyl)-3-[5-(2,2-dimethyl-propyl)-2-(2H-[1,2,3]triazol-4-yl)-benzylamino]-2-hydroxy-propyl}-acetamide, N-(1-(3,5-difluorophenyl)-3-hydroxy-4-(5-neopentyl-2-(pyridin-3-yl)benzylamino)butan-2-yl)acetamide, N-(1-(3,5-difluorophenyl)-4-(2-(6-fluoropyridin-3-yl)-5-neopentylbenzylamino)-3-hydroxybutan-2-yl)acetamide, N-(4-(2-(3-acetylth iophen-2-yl)-5-neopentylbenzylamino)-1-(3,5-difluorophenyl)-3-hydroxybutan-2-yl)acetamide, N-{1-(3,5-difluoro-benzyl)-3-[5-(2,2-dimethyl-propyl)-2-(2-fluoro-pyridin-3-yl)-benzylamino]-2-hydroxy-propyl}-acetamide, N-{1-(3,5-difluoro-benzyl)-3-[5-(2,2-dimethyl-propyl)-2-pyridazin-3-yl-benzylamino]-2-hydroxy-propyl}-acetamide, N-{1-(3,5-difluoro-benzyl)-3-[5-(2,2-dimethyl-propyl)-2-pyrimidin-5-yl-benzylamino]-2-hydroxy-propyl}-acetamide, N-(1-(3,5-difluoro-benzyl)-3-{1-[5-(2,2-dimethyl-propyl)-2-imidazol-1-yl-phenyl]-cyclopropylamino}-2-hydroxy-propyl)-acetamide, N-{1-(3,5-difluoro-benzyl)-3-[5-(2,2-dimethyl-propyl)-2-pyrazin-2-yl-benzylamino]-2-hydroxy-propyl}-acetamide, N-{1-(3,5-difluoro-benzyl)-3-[5-(2,2-dimethyl-propyl)-2-(5-ethyl-imidazol-1-yl)-benzylamino]-2-hydroxy-propyl}-acetamide, N-[3-[3-chloro-5-(2,2-dimethyl-propyl)-2-imidazol-1-yl-benzylamino]-1-(3,5-difluoro-benzyl)-2-hydroxy-propyl]-acetamide, N-{1-(3,5-difluoro-benzyl)-3-[5-(2,2-dimethyl-propyl)-2-tetrazol-1-yl-benzylamino]-2-hydroxy-propyl}-acetamide, N-(1-(3,5-difluorophenyl)-4-(2-(3,5-dimethylisoxazol-4-yl)-5-neopentylbenzylamino)-3-hydroxybutan-2-yl)acetamide, N-{1-(3,5-Difluoro-benzyl)-2-hydroxy-3-[1-(3-th iazol-2-yl-phenyl)-cyclopropylamino]-propyl}-acetamide, N-{1-(3,5-Difluoro-benzyl)-3-[2-(2,2-dimethyl-propyl)-5-(4-hydroxymethyl-imidazol-1-yl)-benzylamino]-2-hydroxy-propyl}-acetamide, N-{1-(3,5-Difluoro-benzyl)-3-[2-(2,2-dimethylpropyl)-5-thiophen-2-yl-benzylamino]-2-hydroxy-propyl}-acetamide, N-[3-[5-(3-Acetyl-thiophen-2-yl)-2-(2,2-dimethyl-propyl)-benzylamino]-1-(3,5-difluoro-benzyl)-2-hydroxy-propyl]-acetamide, N-{1-(3,5-Difluoro-benzyl)-3-[2-(2,2-dimethyl-propyl)-5-fu ran-3-yl-benzylamino]-2-hydroxy-propyl}-acetamide, N-{1-(3,5-Difluoro-benzyl)-3-[2-(2,2-dimethyl-propyl)-5-furan-2-yl-benzylamino]-2-hydroxy-propyl}-acetamide, N-{1-(3,5-Difluoro-benzyl)-3-[2-(2,2-dimethyl-propyl)-5-(1H-pyrrol-2-yl)-benzylamino]-2-hydroxy-propyl}-acetamide, N-{1-(3,5-Difluoro-benzyl)-3-[2-(2,2-dimethyl-propyl)-5-(4-methyl-thiophen-2-yl)-benzylamino]-2-hydroxy-propyl}-acetamide, N-{1-(3,5-Difluoro-benzyl)-3-[2-(2,2-dimethyl-propyl)-5-thiophen-3-yl-benzylamino]-2-hydroxy-propyl}-acetamide, N-[3-[5-Benzofuran-2-yl-2-(2,2-dimethyl-propyl)-benzylamino]-1-(3,5-difluoro-benzyl)-2-hydroxy-propyl]-acetamide, N-[3-[5-Benzo[b]thiophen-2-yl-2-(2,2-dimethyl-propyl)-benzylamino]1-(3,5-difluoro-benzyl)-2-hydroxy-propyl-acetamide, N-{1-(3,5-Difluoro-benzyl)-3-[2-(2,2-dimethyl-propyl)-5-(1-propyl-1H-pyrazol-4-yl)-benzylamino]-2-hydroxy-propyl}-acetamide, N-{1-(3,5-Difluoro-benzyl)-3-[2-(2,2-dimethyl-propyl}-5-(2-formyl-thiophen-3-yl)-benzylamino]-2-hydroxy-propyl]-acetamide, N-{1-(3,5-Difluoro-benzyl)-3-[2-(2,2-dimethyl-propyl)-5-(5-formyl-thiophen-2-yl)-benzylamino]-2-hydroxy-propyl}-acetamide, N-(1-(3,5-difluorophenyl)-3-hydroxy-4-(1-(2-(thiazol-2-yl)phenyl)cyclopropylamino)butan-2-yl)acetamide, N-(1-(3,5-difluorophenyl)-3-hydroxy-4-(5-neopentyl-2-(thiophen-2-yl)benzylamino)butan-2-yl)acetamide, N-(4-(2-(5-acetylthiophen-2-yl)-5-neopentylbenzylamino)-1-(3,5-difluorophenyl)-3-hydroxybutan-2-yl)acetamide, N-(1-(3,5-difluorophenyl)-4-(2-(furan-3-yl)-5-neopentylbenzylamino)-3-hydroxybutan-2-yl)acetamide, N-(1-(3,5-difluorophenyl)-4-(2-(furan-2-yl)-5-neopentylbenzylamino)-3-hydroxybutan-2-yl)acetamide, N-(1-(3,5-difluorophenyl)-3-hydroxy-4-(5-neopentyl-2-(thiophen-3-yl)benzylamino)butan-2-yl)acetamide, N-(1-(3,5-difluorophenyl)-3-hydroxy-4-(2-(4-methylthiophen-2-yl)-5-neopentylbenzylamino)butan-2-yl)acetamide, N-(4-(2-(benzofuran-2-yl)-5-neopentylbenzylamino)-1-(3,5-difluorophenyl)-3-hydroxybutan-2-yl)acetamide, N-(1-(3,5-difluorophenyl)-3-hydroxy-4-(5-neopentyl-2-(1-propyl-1H-pyrazol-4-yl)benzylamino)butan-2-yl)acetamide, N-(4-(2-(1H-indol-2-yl)-5-neopentylbenzylamino)-1-(3,5-difluorophenyl)-3-hydroxybutan-2-yl)acetamide, N-(1-(3,5-difluorophenyl)-3-hydroxy-4-(2-(1-methyl-1H-pyrazol-4-yl)-5-neopentylbenzylamino)butan-2-yl)acetamide, N-(1-(3,5-difluorophenyl)-3-hydroxy-4-(5-neopentyl-2-(1H-pyrazol-4-yl)benzylamino)butan-2-yl)acetamide, N-(1-(3,5-difluorophenyl)-3-hydroxy-4-(2-(5-methylthiophen-2-yl)-5-neopentylbenzylamino)butan-2-yl)acetamide, N-(1-(3,5-difluorophenyl)-4-(2-(2-formylthiophen-3-yl)-5neopentylbenzylamino)-3-hydroxybutan-2-yl)acetamide, N-(1-(3,5-difluorophenyl)-4-(2-(5-formylthiophen-2-yl)-5-neopentylbenzylamino)-3-hydroxybutan-2-yl)acetamide, N-(4-(2-(benzo[b]thiophen-2-yl)-5-neopentylbenzylamino)-1-(3,5-difluorophenyl)-3-hydroxybutan-2-yl)acetamide, N-{1-(3,5-difluoro-benzyl)-3-[5-(2,2-dimethyl-propyl)-2-imidazol-1-yl-benzylamino]-2-hydroxy-propyl}-acetamide, N-(1-(3,5-difluorophenyl)-3-hydroxy-4-(2-(4-methyl-1H-imidazol-1-yl)-5-neopentylbenzylamino)butan-2-yl)acetamide, N-(1-(3,5-difluorophenyl)-3-hydroxy-4-(5-neopentyl-2-(4-phenyl-1H-imidazol-1-yl)benzylamino)butan-2-yl)acetamide, N-(4-(2-(1H-benzo[d]imidazol-1-yl)-5-neopentylbenzylamino)-1-(3,5-difluorophenyl)-3-hydroxybutan-2-yl)acetamide, N-(4-(2-(3-acetyl-1H-pyrrol-1-yl)-5-neopentylbenzylamino)-1-(3,5-difluorophenyl)-3-hydroxybutan-2-yl)acetamide, 1-(2-((3-acetamido-4-(3,5-difluorophenyl)-2-hydroxybutylamino)methyl)-4-neopentylphenyl)-1H-imidazole-4-carboxylic acid, N-{1-(3,5-Difluoro-benzyl)-3-[5-(2,2-dimethyl-propyl)-2-indol-1-yl-benzylamino]-2-hydroxy-propyl}-acetamide, N-(4-(2-(1H-indol-1-yl)-5-neopentylbenzylamino)-1-(3,5-difluorophenyl)-3-hydroxybutan-2-yl)acetamide, N-(1-(3,5-difluorophenyl)-3-hydroxy-4-(5-neopentyl-2-(1H-pyrazol-1-yl)benzylamino)butan-2-yl)acetamide, N-(4-(2-(3-acetyl-1H-pyrazol-1-yl)-5-neopentylbenzylamino)-1-(3,5-difluorophenyl)-3-hydroxybutan-2-yl)acetamide, N-(1-(3,5-difluorophenyl)-3-hydroxy-4-(2-(3-methyl-1H-pyrazol-1-yl)-5-neopentylbenzylamino)butan-2-yl)acetamide, N-{1-(3,5-Difluoro-benzyl)-3-[5-(2,2-dimethyl-propyl)-2-(4-methyl-pyrazol-1-yl)-benzylamino]-2-hydroxy-propyl}-acetamide, N-(4-(2-(1H-indazol-1-yl)-5-neopentylbenzylamino)-1-(3,5-difluorophenyl)-3-hydroxybutan-2-yl)acetamide, N-(1-(3,5-difluorophenyl)-3-hydroxy-4-(5-neopentyl-2-(1H-1,2,3-triazol-1-yl)benzylamino)butan-2-yl)acetamide, N-(1-(3,5-difluorophenyl)-3hydroxy-4-(5-neopentyl-2-(2H-1,2,3-triazol-2-yl)benzylamino)butan-2-yl)acetamide, N-(1-(3,5-difluorophenyl)-3-hydroxy-4-(5-neopentyl-2-(1H-1,2,4-triazol-1-yl)benzylamino)butan-2-yl)acetamide, N-(1-(3,5-difluorophenyl)-3-hydroxy-4-(5-neopentyl-2-(pyrrolidin-1-yl)benzylamino)butan-2-yl)acetamideN-(1-(3,5-difluorophenyl)-3-hydroxy-4-(2-(2-mercapto-1H-imidazol-1-yl)-5-neopentylbenzylamino)butan-2-yl)acetamide, methyl 3-(1-(2-((3-acetamido-4-(3,5-difluorophenyl)-2-hydroxybutylamino)methyl)-4-neopentylphenyl)-1H-imidazol-4-yl)acrylate, 3-(1-(2-((3-acetamido-4-(3,5-difluorophenyl)-2-hydroxybutylamino)methyl)-4-neopentylphenyl)-1H-imidazol-4-yl)-2-aminopropanoic acid, N-(1-(3,5-difluorophenyl)-3-hydroxy-4-(2-(3-hydroxypyrrolidin-1-yl)-5-neopentylbenzylamino)butan-2-yl)acetamide, and N-(1-(3,5-difluorophenyl)-3-hydroxy-4-(5-neopentyl-2-(piperidin-1-yl)benzylamino)butan-2-yl)acetamide, or a pharmaceutically acceptable salt thereof.

6. The method according to claim 1, wherein the aspartyl protease is beta-secretase and the condition is Alzheimer's disease, Down's syndrome or Trisomy 21, hereditary cerebral hemorrhage with amyloidosis of the Dutch type, chronic inflammation due to amyloidosis, prion diseases, Familial Amyloidotic Polyneuropathy, cerebral amyloid angiopathy, degenerative dementias, dementia associated with Parkinson's disease, dementia associated with progressive supranuclear palsy and dementia associated with cortical basal degeneration, diffuse Lewy body type of Alzheimer's disease, and frontotemporal dementias with parkinsonism.

7. The method according to claim 1 wherein the at least one compound of formula (I), 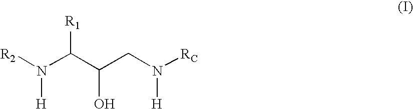
inhibits production of A-beta by at least 10% for a dose of ≦100 mg/kg.

8. The method according to claim 7, wherein the at least one compound of formula (I) is N-{1-(3,5-Difluoro-benzyl)-3-[5-(2,2-dimethyl-propyl)-2-imidazol-1-yl-benzylamino]-2-hydroxy-propyl}-acetamide.

9. The method according to claim 7, wherein the condition is Alzheimer's disease and the at least one aspartyl protease is beta-secretase.

10. The method according to claim 7, wherein the condition is dementia and the at least one aspartyl protease is beta-secretase.

11. A method according to claim 1 wherein the at least one compound of formula (I), 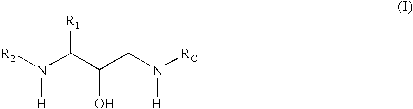
is selective.

12. The method according to claim 11, wherein the at least one compound of formula (I) is chosen from N-(4-(2-(1H-imidazol-1-yl)-5-neopentylbenzylamino)-1-(3,5-difluorophenyl)-3-hydroxybutan-2-yl)acetamide, N-(1-(3,5-difluorophenyl)-3-hydroxy-4-(2-(4-(hydroxymethyl)-1H-imidazol-1-yl)-5-neopentylbenzylamino)butan-2-yl)acetamide, N-{1-(3,5-Difluoro-benzyl)-3-[5-(2,2-dimethyl-propyl)-2-(4-methyl-imidazol-1-yl)-benzylamino]-2-hydroxy-propyl}-acetamide, N-[3-[2-Benzoimidazol-1-yl-5-(2,2-dimethyl-propyl)-benzylamino]-1-(3,5-difluoro-benzyl)-2-hydroxy-propyl]-acetamide, 1-[2-{[3-Acetylamino-4-(3,5-difluoro-phenyl)-2-hydroxy-butylamino]-methyl}-4-(2,2-dimethyl-propyl)-phenyl]-1H-imidazole-4-carboxylic acid, N-(1-(3,5-difluorophenyl)-3-hydroxy-4-(5-neopentyl-2-(2H-1,2,3-triazol-2-yl)benzylamino)butan-2-yl)acetamide, and N-(1-(3,5-difluorophenyl)-3-hydroxy-4-(5-neopentyl-2-(1H-pyrazol-4-yl)benzylamino)butan-2-yl)acetamide .

13. The method according to claim 11, wherein the condition is Alzheimer's disease and the at least one aspartyl protease is beta-secretase.

14. The method according to claim 11, wherein the condition is dementia and the at least one aspartyl protease is beta-secretase.

15. A method according to claim 1 wherein the at least one compound of formula (I), 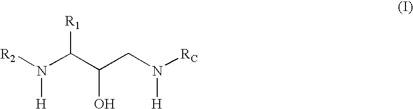
has an F value of at least 10%.

16. The method according to claim 15, wherein the at least one compound of formula (I) is N-(4-(2-(1H-imidazol-1-yl)-5-neopentylbenzylamino)-1-(3,5-difluorophenyl)-3-hydroxybutan-2-yl)acetamide.

17. The method according to claim 15, wherein the condition is Alzheimer's disease and the at least one aspartyl protease is beta-secretase.

18. The method according to claim 15, wherein the condition is dementia and the at least one aspartyl protease is beta-secretase.
